(19) United States
(12) Patent Application Publication
(10) Pub. No.: US 2024/0302378 A1
(43) Pub. Date: Sep. 12, 2024

(54) Methods of Microfluidic Assay and Bioproduction from Non-Mammalian Cells and Kits Therefor

(71) Applicant: Bruker Cellular Analysis, Inc., Emeryville, CA (US)

(72) Inventors: Or Gadish, Richmond, CA (US); Kellen C. Mobilia, Livermore, CA (US); Sara Tafoya, Hayward, CA (US); Eric K. Sackmann, Berkeley, CA (US); Alexander J. Mastroianni, Alameda, CA (US); Peyton Shieh, Emeryville, CA (US); Smriti Sridhar, Berkeley, CA (US); Pierre Sphabmixay, Boston, MA (US); Eva Y. Cheung, Palo Alto, CA (US); Grayson T. Wawrzyn, Oakland, CA (US); Long V. Le, San Francisco, CA (US); Nathan J. Ver Heul, Oakland, CA (US); Bo Hu, Emeryville, CA (US); Lily Chao, Emeryville, CA (US); Ke-Chih Lin, Richmond, CA (US); Volker L.S. Kurz, Oakland, CA (US); Robert M. Onorato, Alameda, CA (US)

(73) Assignee: Bruker Cellular Analysis, Inc., Emeryville, CA (US)

(21) Appl. No.: 18/481,448

(22) Filed: Oct. 5, 2023

Related U.S. Application Data
(63) Continuation of application No. PCT/US2022/023598, filed on Apr. 6, 2022.
(60) Provisional application No. 63/324,566, filed on Mar. 28, 2022.
(60) Provisional application No. 63/171,378, filed on Apr. 6, 2021.
(60) Provisional application No. 63/171,361, filed on Apr. 6, 2021.

(51) Int. Cl. G01N 33/58; C12M 1/34; C12M 3/06

(52) U.S. Cl. CPC G01N 33/582; C12M 23/16; C12M 41/46

(57) ABSTRACT

Metabolic engineering has developed microbial cell factories as sustainable alternatives to chemical synthesis from petroleum feedstocks or harvesting of animals and plants, but current methods can be a costly and labor-intensive commitment. Methods are described herein for microfluidic methods of screening thousands of variant cells in order to reduce time and uncertainty to provide improved strains.

This application is a continuation of International Application No. PCT/US2022/023598, filed Apr. 6, 2022, which claims the benefit under 35 U.S.C. 119(e) of U.S. Provisional Application No. 63/171,361, filed on Apr. 6, 2021, U.S. Provisional Application No. 63/171,378, filed on Apr. 6, 2021, and U.S. Provisional Application No. 63/324,566, filed on Mar. 28, 2022, each of which disclosures is herein incorporated by reference in its entirety.

### BACKGROUND OF THE INVENTION

The industrial synthetic biology sector has made huge investments to achieve relevant miniaturized screening systems for scalable fermentation. Metabolic engineering has developed microbial cell factories as sustainable alternatives to chemical synthesis from petroleum feedstocks or harvesting of animals and plants. These fermentation processes employ natural and engineered enzymes to give one-pot alternatives to conventional manufacturing. The current method most often employed for selection of a microbial strain for manufacturing is the lab-scale bioreactor, which represents a costly and labor-intensive commitment, as thousands or more candidate genotypes are often considered.

### SUMMARY OF THE INVENTION

In a first aspect, a method for evaluating bioproductivity of a cell is provided, the method including: disposing a cell into a chamber of a microfluidic device, the microfluidic device having a microfluidic circuit including a flow region and the chamber, wherein the chamber includes an opening to the flow region; forming an in situ-generated barrier within the chamber, wherein the in situ-generated barrier defines an enclosed culture area within the chamber for culturing the cell; allowing the cell to secrete an analyte within the enclosed culture area; introducing a first fluidic medium including a reporter molecule into the flow region of the microfluidic circuit, wherein the reporter molecule is configured to bind to the analyte to form a reporter molecule: secreted analyte complex (RMSA complex), wherein the reporter molecule includes a first detectable label; and detecting a first signal associated with the first detectable label within an area of interest within the microfluidic circuit, thereby evaluating the bioproductivity of the cell.

In another aspect, a method for evaluating bioproductivity of a cell is provided, the method including: disposing the cell into a chamber of a microfluidic device, the microfluidic device having a microfluidic circuit including a flow region and the chamber, wherein the chamber including an opening to the flow region; forming an in situ-generated barrier within the chamber, wherein the in situ-generated barrier defines within the chamber an assay area and an enclosed culture area for culturing the cell; disposing a micro-object in the assay area of the chamber, wherein the micro-object includes a capture moiety configured to bind an analyte secreted by the cell; allowing the cell to secreting the analyte; introducing a first fluidic medium including a reporter molecule into the flow region, wherein the reporter molecule includes a first detectable label and a binding component configured to bind to the analyte; and detecting a first signal associated with the first detectable label within an area of interest within the microfluidic circuit, thereby evaluating the bioproductivity of the cell.

In a further aspect, a kit for evaluating bioproductivity of a cell is provided, the kit including: a reporter molecule including a first detectable label and a binding component configured to bind an analyte secreted by a cell to form a reporter molecule: secreted analyte complex (RMSA complex); and a prepolymer configured to be controllably activated to form an in situ-generated barrier including a solidified polymer network, wherein the in situ-generated barrier has a porosity that substantially prevents the cell from crossing through the in situ-generated barrier.

In yet another aspect, a method for biomass measurement in a microfluidic device is provided, the method including: obtaining a microfluidic device including a chamber having a biomass to be measured, wherein the microfluidic device includes a microfluidic circuit including a flow region and a chamber fluidically connected to the flow region, wherein the chamber includes an opening to the flow region; obtaining a first brightfield image of the chamber or a first area thereof including the biomass; and measuring a first optical density score from the first brightfield image.

In another aspect, a method for improving yeast cell bioproductivity is provided, the method including; culturing one or more yeast cells within each of a plurality of chambers of a microfluidic device, where the microfluidic device comprises a flow region configured to flow a first fluidic medium and the chamber opens to the flow region; expanding the one or more yeast cells to form a population of yeast cells in each of the plurality of chambers; monitoring the production of a biomolecule by the population of yeast cells in each of the plurality of chambers; and predicting one or more populations of yeast cells configured to effectively produce the biomolecules.

In another aspect, a method is provided for assessing relative productivity of a detectable molecule by a population of yeast cells in a microfluidic device having an enclosure comprising a channel and a plurality of chambers, each chamber of the plurality having an opening fluidically connecting the chamber to the channel, the method including: disposing a yeast cell configured to produce the detectable molecule in each chamber of the plurality of chambers; flowing a first aqueous medium into the channel; culturing the yeast cell to expand to a clonal population of yeast cells; flowing a water immiscible fluidic medium into the channel, displacing substantially all of the first aqueous medium in the channel; monitoring over a period of time an increase of a signal from the detectable molecules produced by the clonal population of yeast cells in each chamber of the plurality; and determining a relative productivity for each clonal population of yeast cells.

In another aspect, a method is provided for assessing relative productivity of a detectable molecule by a population of yeast cells, in a microfluidic device having an enclosure comprising a channel and a plurality of chambers, each chamber of the plurality having an opening fluidically connecting the chamber to the channel, the method including: disposing a yeast cell configured to produce the detectable molecule in each chamber of the plurality of chambers; perfusing an aqueous medium through the channel; culturing the yeast cell to expand to a clonal population of yeast cells; increasing a rate of perfusing the aqueous medium for a selected first period of time thereby establishing a substantially steady state of diffusion of the detectable molecules from each chamber into the channel; imaging a signal from the detectable molecules produced by the clonal population of yeast cells in each chamber of the plurality; and determining a relative productivity for each clonal population of yeast cells.

In another aspect, a method for distributing biological micro-objects in a microfluidic device is provided, where the microfluidic device includes a flow region including a flow channel and at least one chamber fluidically connecting to the flow channel; where the method includes: disposing at least one biological micro-object within a chamber; incubating the at least one biological micro-object thereby forming a population of cells; and centrifuging the microfluidic device to redistribute at least a portion of the population of cells within the microfluidic device.

In another aspect, a method for distributing biological micro-objects in a microfluidic device is provided, including: providing a microfluidic device; where the microfluidic device includes a flow region including a flow channel and at least one chamber fluidically connecting to the flow channel; where at least one biological micro-object is disposed at a first location within the microfluidic device; and centrifuging the microfluidic device to redistribute the at least one biological micro-object from the first region to a second location within the microfluidic device.

### BRIEF DESCRIPTION OF THE DRAWINGS

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A to 2B illustrate a microfluidic device having sequestration pens according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A to 4B illustrate electrokinetic features of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIG. 6 is a schematic representation of a workflow for assessing level of production of an analyte from a cell or colony of cells.

FIG. 7A is a graphical representation of hydrogel barriers according to some embodiments of the disclosure.

FIGS. 7B-7C are photographic representations of hydrogel barriers according to some embodiments of the disclosure.

FIGS. 8A-8C are graphical representations of assay methods utilizing hydrogel barriers according to some embodiments of the disclosure.

FIGS. 9A-9B are graphical representations of OD scores and CV thereof, according to some embodiments of the disclosure.

FIGS. 10A-10B are photographic representations of fluorescent images of microfluidic chambers at two different timepoints, according to some embodiments of the disclosure.

FIGS. 11A-11B are photographic representations of fluorescent and brightfield images of a bead-based assay according to some embodiments of the disclosure.

FIGS. 12A-12B are graphical representations of assay conditions across entire microfluidic chips according to some embodiments of the disclosure.

FIGS. 13A-13F are photographic representations of defined areas of interest for biomass measurements or corrections thereof according to some embodiments of the disclosure.

FIGS. 14A-14B are photographic representations of Bright-field density biomass measurement showing an image of a chip before and after centrifugation packing, illustrating the effect of packing on colony area and bright-field image density.

FIG. 14C is a graphical representation (upper panel) of the relationship between unpacked colony OD score and packed colony area, illustrating assay linearity for the corrected biomass measurement. The lower panel shows the relative variability (coefficient of variation, CV) of biomass sample measurement as a function of colony size, assuming perfect linearity of post-packing colony area with biomass.

FIG. 15 is a graphical representation of variability measured for fluorescence detection of product using standard solutions perfused through the OptoSelect chip. Every chamber in the chip was included in the measurement (n˜3500 for each).

FIGS. 16A-B are photographic representations of fluorescent images of chambers with barriers with size-dependent permeability according to some embodiments of the disclosure.

FIGS. 17A-17C are photographic representation of fluorescent images of chamber with size dependent permeability according to some embodiments of the disclosure.

FIGS. 18A-18C are brightfield photographic representations of cells culturing according to some embodiments of the disclosure.

FIGS. 18D-18F are photographic representations of fluorescent images of assays according to some embodiments of the disclosure.

FIG. 19 is a graphical representation of results of productivity assays according to some embodiments of the disclosure.

FIG. 20 is a graphical representation of results of productivity assays according to some embodiments of the disclosure

FIG. 21 is a graphical representation of the distribution of productivity assay results according to some embodiments of the disclosure.

FIG. 22 is a graphical representation of results of productivity assay results according to some embodiments of the disclosure.

FIG. 23 is a graphical representation of results of productivity assay results according to some embodiments of the disclosure.

FIG. 24 is a graphical representation of comparison of raw and corrected assay results according to some embodiments of the disclosure.

FIGS. 25A-25F are photographic representations with annotation showing the process of exporting cells from a chamber having a non-uniform hydrogel barrier according to some embodiments of the disclosure.

FIG. 26A shows a photographic representation of one embodiments of a sealed chamber assay, taken over time and including fluorescent images.

FIG. 26B shows graphical representations of biomass normalized time-course plots of fluorescence during the assay period for three strains. Each individual narrow weight line represents an individual pen, with the average value for each strain indicated by the bolded line.

FIG. 26C shows the correlation between average sealed pen assay scores qₚ with the average lab scale bioreactor qₚ measuring during the 24-48 hour interval. The qₚ score for the pens in the microfluidic device was calculated by normalizing the average rate of increase in fluorescence to the OD score (corresponding to the slopes of the faint lines in b). Bioreactor data plotted are the mean of several biological replicates per strain (n=2-5). Error bars in both directions show the 95% confidence interval of the mean. The least-squares linear regression line (blue) and its 95% confidence band (gray) are also shown (R²=0.87, p=0.006).

FIG. 27 illustrates graphical representations of sealed pen assay results, showing time-lapse biomass and fluorescence on three replicate microfluidic devices. Each narrow line corresponds to a single chamber of the microfluidic device, each broad line represents the average of all chambers housing the same strain within the microfluidic device. Biomass is captured by the top row (OD metric), while total accumulated product fluorescence in a chamber is shown in the bottom row (Intensity metric).

FIG. 28 is a graphical representation of the correlation between closed chamber assay scores and the open chamber assay scores (R²=098). The error bars represent the standard deviation of scores of each strain.

FIGS. 29A to 29C are graphical representations of a diffusion gradient assay according to some embodiments of the disclosure.

FIG. 30 is a cross-section of a chamber of a micro-fluidic device showing the region of interest and subregions thereof of a diffusion gradient assay according to some embodiments of the disclosure.

FIG. 31 is a schematic representation of a workflow for assessing an intrinsic diffusion gradient.

### DETAILED DESCRIPTION OF THE INVENTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one. As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, “air” refers to the composition of gases predominating in the atmosphere of the earth. The four most plentiful gases are nitrogen (typically present at a concentration of about 78% by volume, e.g., in a range from about 70-80%), oxygen (typically present at about 20.95% by volume at sea level, e.g. in a range from about 10% to about 25%), argon (typically present at about 1.0% by volume, e.g. in a range from about 0.1% to about 3%), and carbon dioxide (typically present at about 0.04%, e.g., in a range from about 0.01% to about 0.07%). Air may have other trace gases such as methane, nitrous oxide or ozone, trace pollutants and organic materials such as pollen, diesel particulates and the like. Air may include water vapor (typically present at about 0.25% or may be present in a range from about 10 ppm to about 5% by volume). Air may be provided for use in culturing experiments as a filtered, controlled composition and may be conditioned as described herein.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 microliters. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light that is modulated to provide one or more illumination effects. A first illumination effect may be projected light illuminating a portion of a surface of a device without illuminating (or at least minimizing illumination of) an adjacent portion of the surface, e.g., a projected light pattern, as described more fully below, used to activate DEP forces within a DEP substrate. When using structured light patterns to activate DEP forces, the intensity, e.g., variation in duty cycle of a structured light modulator such as a DMD, may be used to change the optical power applied to the light activated DEP actuators, and thus change DEP force without changing the nominal voltage or frequency. Another illumination effect that may be produced by structured light includes projected light that may be corrected for surface irregularities and for irregularities associated with the light projection itself, e.g., fall-off at the edge of an illuminated field. Structured light is typically generated by a structured light modulator, such as a digital mirror device (DMD), a microshutter array system (MSA), a liquid crystal display (LCD), or the like. Illumination of a small area of the surface, e.g., a selected area of interest, with structured light improves the signal-to-noise-ratio (SNR), as illumination of only the selected area of interest reduces stray/scattered light, thereby lowering the dark level of the image. An important aspect of structured light is that it may be changed quickly over time. A light pattern from the structured light modulator, e.g., DMD, may be used to autofocus on difficult targets such as clean mirrors or surfaces that are far out of focus. Using a clean mirror, a number of self-test features may be replicated such as measurement of modulation transfer function and field curvature/tilt, without requiring a more expensive Shack-Hartmann sensor. In another use of structured light patterns, spatial power distribution may be measured at the sample surface with a simple power meter, in place of a camera. Structured light patterns may also be used as a reference feature for optical module/system component alignment as well used as a manual readout for manual focus. Another illumination effect made possible by use of structured light patterns is selective curing, e.g., solidification of hydrogels within the microfluidic device.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, glass beads, amorphous solid substrates, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, proteins (including receptor molecules), carbohydrates, antigens, small molecule signaling moieties, or other chemical/biological species capable of use in an assay. In some variations, beads/solid substrates including moieties/molecules may be capture beads, e.g., configured to bind molecules including small molecules, peptides, proteins or nucleic acids present in proximity either selectively or nonselectively. In one nonlimiting example, a capture bead may include a nucleic acid sequence configured to bind nucleic acids having a specific nucleic acid sequence or the nucleic acid sequence of the capture bead may be configured to bind a set of nucleic acids having related nucleic acid sequences. Either type of binding may be understood to be selective. Capture beads containing moieties/molecules may bind nonselectively when binding of structurally different but physico-chemically similar molecules is performed, for example, size exclusion beads or zeolites configured to capture molecules of selected size or charge. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g., about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

As referred to herein, “gas permeable” means that the material or structure is permeable to at least one of oxygen, carbon dioxide, or nitrogen. In some embodiments, the gas permeable material or structure is permeable to more than one of oxygen, carbon dioxide and nitrogen and may further be permeable to all three of these gases.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion, and may encompass perfusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result. Flowing can comprise pulling solution through and out of the microfluidic channel (e.g., aspirating) or pushing fluid into and through a microfluidic channel (e.g., perfusing).

The phrase “substantially no flow” refers to a rate of flow of a fluidic medium that, when averaged over time, is less than the rate of diffusion of components of a material (e.g., an analyte of interest) into or within the fluidic medium. The rate of diffusion of components of such a material can depend on, for example, temperature, the size of the components, and the strength of interactions between the components and the fluidic medium.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

As used herein, a “flow path” refers to one or more fluidically connected circuit elements (e.g., channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

As used herein, “pen” or “penning” refers to disposing micro-objects within a chamber (e.g., a sequestration pen) within the microfluidic device. Forces used to pen a micro-object may be any suitable force as described herein such as dielectrophoresis (DEP), e.g., an optically actuated dielectrophoretic force (OEP); gravity; magnetic forces; or tilting. In some embodiments, penning a plurality of micro-objects may reposition substantially all the micro-objects. In some other embodiments, a selected number of the plurality of micro-objects may be penned, and the remainder of the plurality may not be penned. In some embodiments, when selected micro-objects are penned, a DEP force, e.g., an optically actuated DEP force or a magnetic force may be used to reposition the selected micro-objects. Typically, micro-objects may be introduced to a flow region, e.g., a microfluidic channel, of the microfluidic device and introduced into a chamber by penning.

As used herein, “unpen” or “unpenning” refers to repositioning micro-objects from within a chamber, e.g., a sequestration pen, to a new location within a flow region, e.g., a microfluidic channel, of the microfluidic device. Forces used to unpen a micro-object may be any suitable force as described herein such as dielectrophoresis, e.g., an optically actuated dielectrophoretic force; gravity; magnetic forces; or tilting. In some embodiments, unpenning a plurality of micro-objects may reposition substantially all the micro-objects. In some other embodiments, a selected number of the plurality of micro-objects may be unpenned, and the remainder of the plurality may not be unpenned. In some embodiments, when selected micro-objects are unpenned, a DEP force, e.g., an optically actuated DEP force or a magnetic force may be used to reposition the selected micro-objects.

As used herein, “export” or “exporting” refers to repositioning micro-objects from a location within a flow region, e.g., a microfluidic channel, of a microfluidic device to a location outside of the microfluidic device, such as a 96 well plate or other receiving vessel. The orientation of the chamber(s) having an opening to the microfluidic channel permits easy export of micro-objects that have been positioned or repositioned (e.g., unpenned from a chamber) to be disposed within the microfluidic channel. Micro-objects within the microfluidic channel may be exported without requiring disassembly (e.g., removal of the cover of the device) or insertion of a tool into the chamber(s) or microfluidic channel to remove micro-objects for further processing.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, a “non-sweeping” rate of fluidic medium flow means a rate of flow sufficient to permit components of a second fluidic medium in an isolation region of the sequestration pen to diffuse into the first fluidic medium in the flow region and/or components of the first fluidic medium to diffuse into the second fluidic medium in the isolation region; and further wherein the first medium does not substantially flow into the isolation region.

The term “equilibrium” as defined herein refers to a state of a system in which the average quantity of one or more species of interest (e.g., reporter, analyte, and/or reporter-analyte (or RMSA) complex) does not change as a function of time. In some instances, the system is a closed system that attains equilibrium from non-equilibrium initial conditions. In other instances, the system is an open system that attains equilibrium when the rate of generation and/or addition of the species of interest to the system is equal to the rate of destruction and/or removal of the species of interest from the system. The term “steady state” as defined herein refers to an equilibrium condition in an open system in which the net change of a species of interest over time is zero. The term “non-equilibrium” as defined herein refers to a state of a system in which the average quantity of one or more species of interest (e.g., reporter, analyte, and/reporter-analyte (or RMSA) complex) changes as a function of time.

The term “intrinsic diffusion gradient” as defined herein refers to a difference in concentration of a species of interest (e.g., reporter, analyte, and/or reporter-analyte (or RMSA) complex) between a first region and a second region within a system in which the species of interest is capable of diffusing between the first region and the second region. For example, the system can have a first region in which the species of interest has a first concentration and a second region in which the species of interest has a second concentration that is less than the first concentration. In some instances, the intrinsic diffusion gradient can arise from the generation of a soluble analyte in a first region of a system, where the system includes a second region in which there is no generation of the soluble analyte (or less generation than in the first region). In some instances, the intrinsic diffusion gradient can arise from continuous generation of a soluble analyte in a first region of the system, diffusion of the soluble analyte from the first region to a second region of the system, and continuous removal of the soluble analyte from the second region of the system. Thus, the first region of the system can contain a “source” of the soluble analyte, and the second region of the system can contain a “sink” for the soluble analyte. If the rate of generation of the soluble analyte by the source remains substantially constant over time, the rate of removal of the soluble analyte by the sink remains substantially constant over time, and the rate of generation is substantially the same as the rate of removal, then a “stable concentration gradient” can form. Alternatively, if the rate of generation of the soluble analyte by the source remains substantially constant over time, the rate of removal of the soluble analyte by the sink remains substantially constant over time, but the rate of generation differs from the rate of removal, then a “transient concentration gradient” can form. In some instances, the intrinsic diffusion gradient is a stable concentration gradient. In other instances, the intrinsic diffusion gradient is a transient concentration gradient.

The terms “region of interest” (or “ROI”) and “area of interest” (or “AOI”) are used interchangeably herein and, when used in reference to the measurement of an intrinsic diffusion gradient, refer to a region where an intrinsic diffusion gradient or a portion of the intrinsic diffusion gradient can be measured. The term “axis of diffusion” as defined herein refers to an axis within a system which is parallel to the predominant direction of flow of a species of interest as it moves down its intrinsic diffusion gradient. In some instances, the region of interest can include one or more regions that lie along an axis of diffusion within the system. In other instances, the region of interest can include one or more regions that lie off of an axis of diffusion within the system.

### I. DIFFUSION GRADIENT ASSAYS (GENERALLY)

Methods for assaying an intrinsic diffusion gradient can be useful for assessing and/or quantifying the secretion of a biomolecule of interest by a biological micro-object (e.g., a cell). Such methods can include detecting soluble molecules (or analytes, reporter molecules, or reporter-analyte complexes) in a microfluidic system (e.g., a microfluidic device). In certain embodiments, the microfluidic system can include a chamber (e.g., a chamber of a microfluidic device) having an opening (e.g., to a larger chamber or to a flow channel of the microfluidic device). One or more biological micro-objects secreting the biomolecule of interest disposed in the chamber can be the source of the biomolecule of interest, while the opening of the chamber provides the sink for removal of biomolecule of interest from the chamber. The biological micro-object can comprise, consist of, or consist essentially of any micro-object configured or capable of secreting, producing, or otherwise generating a secreted biomolecule of interest. In some embodiments, the biological micro-object is a cell or a population of cells (e.g., a clonal population). An intrinsic diffusion gradient can be formed in such a system and, as described further herein, measured in such a system.

In some instances, the methods of assaying an intrinsic diffusion gradient include capturing images of reporter molecules (e.g., molecules capable of being detected by an image through emission or absorption of electromagnetic energy typically in the form of photons). The reporter molecule can be configured to emit a detectable signal (e.g., intrinsically or via a detectable label) and include a binding component that binds the secreted biomolecule to be quantified. In some instances, the intrinsic diffusion gradient can be detected in one or more images as signals which can be correlated with the soluble biomolecules of interest that form the intrinsic diffusion gradient. Such signals, for example, can have spatial and/or temporal distributions that are informative of one or more properties of the intrinsic diffusion gradient and, thus, the quantity of the biomolecule of interest being secreted by the biological micro-object(s).

Systems and methods for performing typical diffusion gradient assays are described in greater detail below. Such systems and methods more fully described, for example, in International Application Serial No. PCT/2017/027795, entitled “Methods, Systems, and Kits for In-Pen Assays”, filed on Apr. 14, 2017, published as International Application Publication WO2017/1811135; International Application Serial No. PCT/US2018/055918, entitled “Methods, Systems, and Kits for In-Pen Assays”, filed on Oct. 15, 2018, published as International Application Publication WO2019/075476; and International Application Serial No. PCT/US2021/021417, entitled “Methods, Systems, and Kits for In-Pen Assays”, filed on Mar. 9, 2020, published as International Application Publication WO2021/184458, the entirety of each of which disclosures are herein incorporated by reference for any purpose.).

Secreted analyte of interest. An analyte of interest (i.e., biomolecule of interest, target protein, etc.) secreted by the biological micro-object(s) may include a protein, a saccharide, a nucleic acid, an organic molecule other than a protein, saccharide, or nucleic acid, or a complex biomolecule formed by any one or more of the foregoing. In some embodiments, the analyte secreted by the biological micro-object may be an antibody. In some embodiments, the analyte secreted by the biological micro-object may be a protein other than an antibody. Whether an antibody or a protein other than an antibody, the secreted analyte of interest may be glycosylated (or not) or otherwise chemically modified (or not).

A secreted analyte of interest may be a naturally expressed analyte (e.g., natively expressed) or may be a bioengineered analyte (e.g., a product resulting from gene insertion, deletion, modification and the like). A secreted analyte of interest that is a nucleic acid may be a ribonucleic or a deoxynucleic acid, and may include natural or unnatural nucleotides. A secreted analyte that is a saccharide may be a mono-, di- or polysaccharide. Non-limiting examples of saccharides may include glucose, trehalose, mannose, arabinose, fructose, ribose, xanthan or chitosan. A secreted small, organic molecule may include but is not limited to biofuels, oils, polymers, or pharmaceutics such as macrolide antibiotics. A secreted analyte of interest that is a protein can be an antibody or fragment thereof; a blood protein, such as an albumin, a globulin (e.g., alpha2-macroglobulin, gamma globulin, beta-2 microglobulin, haptoglobulin), a complement protein (e.g., component 3 or 4), transferrin, prothrombin, alpha 1 antitrypsin, and the like; a hormone, such as insulin, glucagon, somatostatin, growth hormone, growth factors (e.g., FGF, HGF, NGF, EGF, PDGF, TGF, Erythropoietin, IGF, TNF), follicle stimulating hormone, luteinizing hormone, leptin, and the like; a fibrous protein, such as a silk or an extracellular matrix protein (e.g., a fibronectin, laminin, collagen, elastin, vitronectin, tenascin, versican, bone sialoprotein); an enzyme, such as a metalloprotease (e.g., matrix metalloproteinase (MMP)) or other type of protease (e.g., serine protease, cysteine protease, threonine protease, aspartic protease, glutamic protease, asparagine peptide lyase), an amylase, a cellulase, a catalase, a pectinase, and the like; a bacterial, yeast, or protozoan protein; a plant protein; or a viral protein, such as a capsid or envelope protein. The secreted analyte may be an antibody-drug conjugate. A non-limiting example of a secreted analyte that may have a combination of a protein, a saccharide, a nucleic acid, an organic molecule having a molecular weight of less than 3 kD, and/or a virus, can include a proteoglycan or glycoprotein. Secreted analyte can comprise an engineered binding site commonly used for purification, said purification tags can include but are not limited to be a structured or unstructured binding domain configured to associate with a reporter molecule. This list is not limiting and any protein that is naturally expressed or may be engineered to be secreted may be evaluated by the disclosed methods of assaying an intrinsic diffusion gradient and/or quantifying a level of secretion of a biological molecule by a biological micro-object(s).

A secreted analyte of interest (e.g., analyte) can comprise a broad range of molecular weights while retaining the ability to diffuse through appropriate media. The secreted analyte of interest can comprise a molecular weight, wherein said molecular weight is proportional to a diffusion rate and therefore correlated with how much (e.g., the concentration) of the secreted analyte that accumulates in the pen under a steady state equilibrium.

Reporter molecules. Methods of assaying an intrinsic diffusion gradient and/or quantifying a level of secretion of a biomolecule of interest by a biological micro-object(s) can comprise the use of one or more reporter molecules (e.g., detection reagents). In certain embodiments, such reporter molecules can be configured to: covalently or non-covalently bind to a secreted analyte of interest; and generate a signal that can be detected (e.g., using imaging). The signal (raw or processed using one or more methods disclosed here in) can provide a direct or indirect measure of diffusion related properties, such as concentration(s) and/or diffusion rate constant(s), which are proportional to the molecular weight of the reporter molecule and/or reporter molecule-secreted analyte (RMSA) complex. See, e.g., International Publication No. WO 2021/183458, published on 16 Sep. 2021, the entire contents of which is incorporated herein by reference. In some embodiments, the signal is proportional to one or more of the amount of accumulated reporter molecule/RMSA complex resulting from one or more of: the secretion rate of a biological micro-object, the number of biological micro-objects, and/or the fraction bound of the analyte.

A reporter molecule typically includes a binding component configured to bind the secreted analyte of interest. Thus, the binding component may be any suitable binding partner capable of specifically binding to the secreted analyte of interest (e.g., with a binding constant less than 10 micromolar). As used herein, specific binding refers to a preference for binding the secreted analyte of interest over one or more other components of the system (e.g., one or more components on or within the microfluidic device). The binding component may comprise a protein, a peptide, a nucleic acid, a small organic molecule, or any combination thereof.

In some embodiments, the reporter molecule may be multi-valent, comprising more than one binding component such that the reporter molecule is able to bind more than one copy of the secreted analyte of interest or to bind more than one member of a group of secreted analytes. The stoichiometry of the reporter molecule-secreted analyte (RMSA) complex can therefore vary. One or more reporter molecules may bind to one or more secreted analytes, and additionally or alternatively one or more secreted analytes may bind to one or more reporter molecules. Thus, for example, a reporter molecule that binds a single copy of the secreted analyte may form an RMSA complex with a 1:1 stoichiometry. Alternatively, the RMSA complex may have a stoichiometric ratio of 2:1, 3:1, 4:1, 1:2, 1:3, 1:4, 2:2, 4:2, 2:4, etc. of reporter molecule: secreted analyte.

The reporter molecule may have any suitable molecular weight, provided that the reporter molecule is soluble and capable of diffusing in media disposed within the microfluidic device. For example, the reporter molecule may have a molecular weight that is about 10%, 20%, 30%, 40%, 50%, 60%, 70%, 80%, 90%, or about the same as the molecular weight of the secreted analyte of interest. Alternatively, the reporter molecule may have a molecular weight that is greater than the molecule weight on the secreted analyte of interest.

In some embodiments, the analyte of interest may be an antibody (or a fragment thereof) and the reporter molecule may comprise a binding component suitable for binding to antibodies (or fragments thereof). In some embodiments, the binding component of the reporter molecule may bind to an antibody Fc region (e.g., the Fc region of an IgG antibody) or an antibody light chain region (e.g., a lambda light chain region or a kappa light chain region). Thus, for example, the binding component of the reporter molecule may include a peptide, protein, aptamer, etc. configured to bind one or more portions/regions of an antibody (e.g., an IgG antibody or fragment thereof). Molecules capable of specifically binding antibodies are well-known in the art. See, e.g., International Publication Nos. WO 2017/181135 and WO 2021/183458.

In some embodiments, the binding component of the reporter molecule may intrinsically possess the ability to generate a detectable signal, such as a visible, luminescent, phosphorescent, or fluorescent signal. In other embodiments, the reporter molecule may comprise a detectable label, which may be covalently attached directly or indirectly to the binding component of the reporter molecule. In other embodiments, the reporter molecule may comprise a detectable label that binds non-covalently to the binding component of the reporter molecule. The detectable label may be a visible, luminescent, phosphorescent, or fluorescent detectable label. In some embodiments, the detectable label may be a fluorescent label. Any suitable fluorescent label may be used, including but not limited to fluorescein, rhodamine, cyanine, phenanthrene or any other class of fluorescent dye.

In some embodiments, the binding component of the reporter molecule may comprise a capture oligonucleotide and the detectable label may be an intercalating dye. For example, the reporter molecule may comprise a capture oligonucleotide and either an intrinsic or extrinsic fluorescent dye may be the detectable label. In some embodiments, the detectable label of the reporter molecule may not be detectable until the capture oligonucleotide binds the analyte of interest, as might be the case when the detectable label is an intercalating dye. More generally, in some embodiments, a detectable label of a reporter molecule may not be detectable until after the RMSA complex has formed, as formation of the RMSA complex may shift the detectable signal to a new wavelength not present prior to binding.

Media. Media suitable for use in methods of assaying an intrinsic diffusion gradient and/or quantifying a level of secretion of a biological molecule by a biological micro-object(s) can be a liquid or a gas, and may comprise reagents (e.g., reporter molecules) or other diffusible components. In various embodiments, the methods may include flowing such a medium (e.g., by stopped flow, continuous flow, pulsed flow, etc., as needed) into a flow region of a microfluidic device. Such flowing (or perfusing) can occur before and/or after introducing biological micro-object(s) into one or more chambers of the microfluidic device.

In certain embodiments, the medium can comprise standard tissue culture components. Exemplary tissue culture components can include: a buffer (e.g., for providing a defined pH and/or ionic strength), dissolved oxygen, one or more soluble stimulatory components, one or more soluble feeder cell components, and/or an exhausted growth medium component. In some embodiments, the amount of dissolved oxygen in the medium may be measured and altered or adjusted as desired, which may be facilitated within the microfluidic environments described herein, as compared to such adjustment in macro-scale culture well plates, shake flasks, and the like. In some embodiments, the pH of the culture medium within the microfluidic environment may be monitored and altered or adjusted, again which may be facilitated within the microfluidic environments described herein, as compared to plasticware standardly used. In some embodiments, soluble stimulatory components such as cytokines, growth factors, antibodies which activate cell-surface signaling proteins, and the like, any of which may stimulate the cells within the microfluidic environment to reproduce more rapidly or to produce different analytes than prior to introduction of the stimulatory components. In some embodiments, viability of the biological micro-object(s) being cultured (e.g., within the microfluidic device) may be improved by including a portion of the supernatant culture medium of feeder cells that provide auxiliary biomolecules that stimulate or otherwise support the cells. The feeder cells themselves may not be present within the microfluidic device but may be cultured in standard reaction vessels. Accordingly, portions of the culture medium conditioned by the feeder cells may be harvested and delivered to the microfluidic device free of the feeder cells. In some embodiments, one or more compounds and/or reagents configured to prevent biological micro-objects from adhering to each other and the chambers may be added to the culture medium. In some embodiments, exhausted growth medium may be added to the microfluidic environment, which can act as a selection mechanism for analyzing which clones within the microfluidic environment are still able to produce the secreted analyte (or even more readily) and/or may be used to approximate the scaleup environment of various types of reaction vessels, which may include well plates, shaker flasks and bioreactors. In some embodiments, one or more of these additions to the culture medium may confer a selective pressure on one or more of the cells within the chambers.

In certain embodiments, the medium can further comprise one or more reporter molecules and/or analytes. In other embodiments, the medium can lack a reporter molecule and/or analyte. For example, in methods involving more than one reporter molecule or analyte, the medium can comprise all or less than all of the reporter molecules and/or analytes.

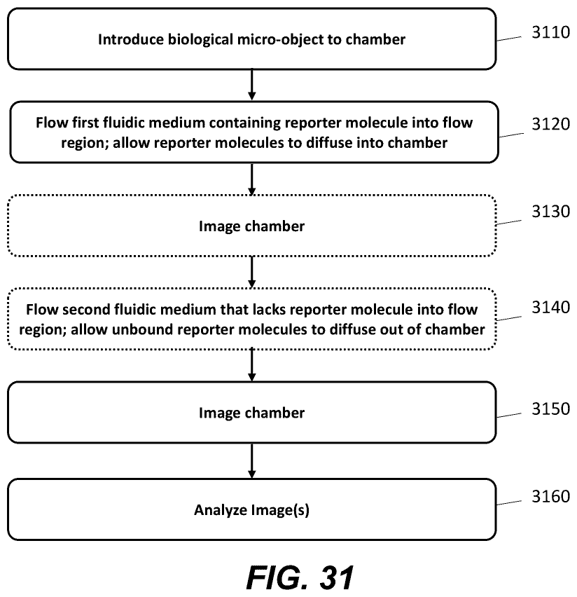

Methods. The disclosed methods for assaying an intrinsic diffusion gradient and/or assessing (e.g., quantifying) a level of secretion of an analyte of interest from a biological micro-object or a population of biological micro-objects (e.g., a clonal population) can include assessing a concentration of a particular diffusible species (e.g., free reporter, reporter bound to secreted analyte, etc.) and/or properties of the species that can change spatially and/or temporally. The disclosed methods can comprise one or more operations (see FIG. 31), which can include but are not limited to: introducing or disposing one or more biological micro-objects (e.g., a biological cell or cells) in each of one or more chambers of a microfluidic device 3110; flowing one or more medium (e.g., as described above or elsewhere herein) into a flow region of the microfluidic device 3120, 3140 (e.g., by continuous flow, pulsed flow, stopped flow, etc.) for a period of time (e.g., a first period of time, a second period of time, a third period of time, etc.), where each of the one or more chambers are fluidically connected and open to the flow region and where each medium may comprise one or more reporter molecules (e.g., a first reporter molecule, a second reporter molecule, a third reporter molecule, etc.); and assessing signal in a region of interest (e.g., a first area of interest, one or more areas along an axis of diffusion, or any location that is free of biological micro-objects), which may be performed by imaging 3130, 3150 for the purpose of detecting a concentration of each of the one or more reporter molecules 3160 (e.g., the concentration of free reporter molecule and/or reporter molecule bound to a secreted analyte of interest, etc.). Each operation can be executed with a particular intent, outcome, or aim, and as evident from the further discussion, certain operations (e.g., 3130, 3140) are optional and can omitted, individually or together, to achieve a particular goal.

In some embodiments, the methods for assaying an intrinsic diffusion gradient and/or assessing (e.g., quantifying) a level of secretion of an analyte of interest from a biological micro-object or a population of biological micro-objects (e.g., a clonal population) can comprise steps for generating background data. Such steps can be performed to measure fluorescence (e.g., autofluorescence) within a system, including in the microfluidic device and/or the media. Background data can comprise background images taken using methods comprising steps to generate said background data. Background data can be used for correction and/or subtraction of fluorescence images taken under non-background conditions. The methods can comprise steps of capturing one or more images using a filter cube relevant to the background and non-background conditions, and taken at a defined exposure time and/or at defined periods of time relative to non-background conditions. In some embodiments, one or more background images may be taken under conditions where medium is flowing into and through the flow region of the microfluidic device. Alternatively, or in addition, one or more background images can be taken under non-flow conditions (e.g., under conditions where medium is present in, but not being actively flowed through, the flow region of the microfluidic device).

In some embodiments, the methods for assaying an intrinsic diffusion gradient and/or assessing (e.g., quantifying) a level of secretion of an analyte of interest from a biological micro-object or a population of biological micro-objects (e.g., a clonal population) can comprise steps for obtaining data under conditions leading to or at an equilibrium state (e.g., a steady-state equilibrium). In some instances, such data can be generated using steps that produce an equilibrium state or steady-state equilibrium conditions within a microfluidic device.

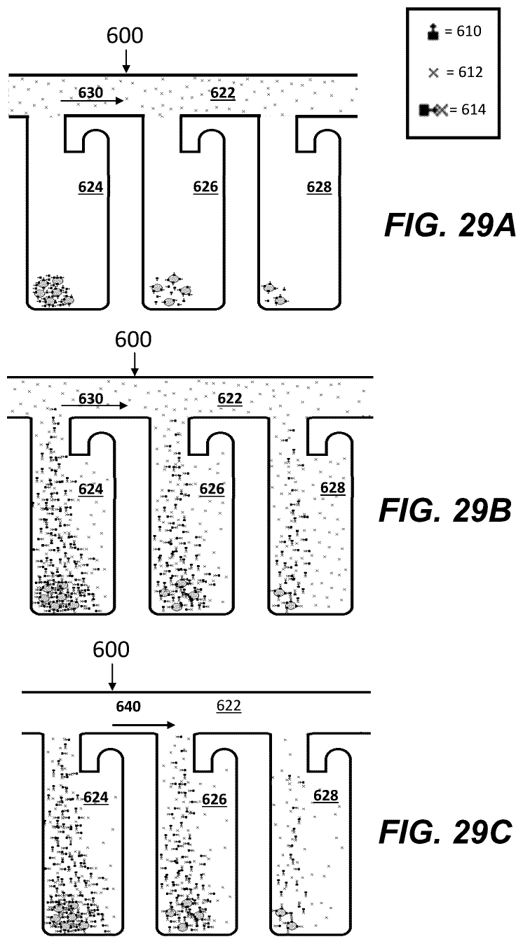

Methods comprising steps for obtaining data under steady-state equilibrium conditions can comprise performing an equilibration assay. See, e.g., FIGS. 29A-29B. In some embodiments, methods that include performing an equilibration assay can comprise disposing a biological micro-object into a chamber 2924 (and/or 2926 and/or 2928) of a microfluidic device (see also 3110 of FIG. 31), where the microfluidic device includes an enclosure having a flow region 2922 and the chamber 2924 is fluidically connected to the flow region 2922, and where the chamber 2924 contains a first fluidic medium; allowing the biological micro-object, or a population of biological micro-objects generated therefrom, to secrete an analyte of interest 2910 into the first fluidic medium within the chamber 2924; introducing a second fluidic medium 2930 into the flow region, where the second fluidic medium contains a plurality of reporter molecules 2912 and allowing a portion of the plurality of reporter molecules 2912 to diffuse into the chamber and bind to the analyte of interest 2912 secreted therein (see also 3120 of FIG. 31), thereby producing a plurality of reporter molecule: secreted analyte (RMSA) complexes 2914; and detecting reporter molecules 2912 located within a region of interest within the microfluidic device 2900 (see also 3130 of FIG. 31), wherein the region of interest includes at least a portion of the chamber 2924. In some embodiments, the first fluidic medium differs from the second fluidic medium. For example, the first fluidic medium may lack reporter molecules 2912, while otherwise sharing the same buffer, pH, level of dissolved oxygen, and/or stimulating factors, etc. In some embodiments, allowing the portion of the plurality of reporter molecules 2912 to diffuse into the chamber 2924 and bind to the analyte of interest 2910 secreted therein is performed for a period of time (e.g., a first period of time) sufficient for unbound reporter molecules to reach a state of equilibrium between the flow region and the chamber. In some embodiments, detecting reporter molecules 2912 in the region of interest comprises detecting unbound reporter molecules 2912 as well as detecting reporter molecules 2912 that are part of RMSA complexes 2914. Reporter molecules 2912 (and RMSA complexes 2914) can be detected within the region of interest via one or more images taken of the associated chamber(s) 2924 (and/or 2926 and/or 2928) and flow region(s) 2922, with the image(s) being analyzed to determine or calculate a score correlated with the level of secreted analyte of interest 2910 in the chamber(s) 2924 (and/or 2926 and/or 2928), as discussed further below and/or elsewhere herein. In various embodiments, methods that include performing an equilibration assay can comprise flowing one or more additional fluidic media (e.g., a third fluidic medium, a fourth fluidic medium, etc.) through the flow region of the microfluidic device, either before detecting the reporter molecules (e.g., in connection with generating background data) or after detecting the reporter molecules (e.g., using a different reporter molecule to further analyze the analyte of interest or to analyze a second analyte of interest).

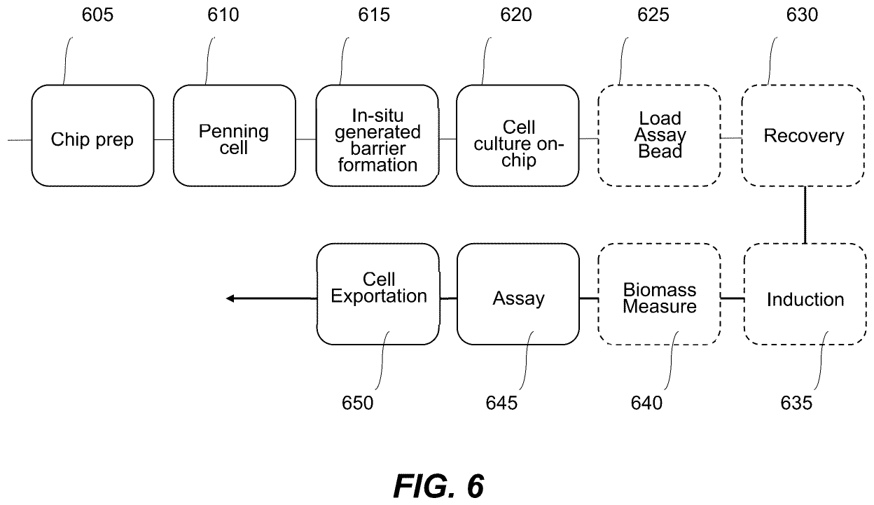

In some embodiments, the methods for assaying an intrinsic diffusion gradient and/or assessing (e.g., quantifying) a level of secretion of an analyte of interest from a biological micro-object or a population of biological micro-objects (e.g., a clonal population) can comprise (or further comprise) steps for performing a flush assay. See, e.g., FIGS. 29A-29C; see also FIG. 31. Such methods may further include: introducing a third fluidic medium 2940 into the flow region 2922, where the third fluidic medium 2940 does not include reporter molecules 2912 and, in some embodiments, allowing at least a portion of unbound reporter molecules 2912 to diffuse out of the chamber 2924 for a period of time (e.g., a second period of time) (see also 3140 of FIG. 31). In some embodiments, the second period of time may be selected based on modelling of diffusion profiles for unbound reporter molecules and RMSA complexes. In some embodiments, introducing the third fluidic medium 2940 into the flow region is performed after allowing a portion of the plurality of reporter molecules 2912 to diffuse into the chamber and bind to the analyte of interest 2910 secreted therein; and, optionally, after allowing unbound reporter molecules 2912 to reach a state of equilibrium between the flow region 2922 and the chamber 2924 (see, e.g., FIG. 6B). In some embodiments, introducing the third fluidic medium 640 into the flow region 622 is performed after detecting reporter molecules 2912 located within a region of interest within the microfluidic device 2900 (see also 3130, 3140 of FIG. 31). Thus, the flush assay can be performed with a single detection step 3150 (i.e., after introducing the third fluidic medium 2940 into the flow region (see 3140 of FIG. 31) but before detecting the reporter molecules 2912 located within the region of interest) or with at least two detecting steps (e.g., with the detection of reporter molecules 2912 in the region of interest occurring both before (see 3130 of FIG. 31) and after (see 3150 of FIG. 31) introducing the third fluidic medium 2940 into the flow region 2922, thereby allowing for a combined equilibration and flush assay).

In some embodiments, the methods for assaying an intrinsic diffusion gradient and/or assessing (e.g., quantifying) a level of secretion of an analyte of interest from a biological micro-object or a population of biological micro-objects (e.g., a clonal population) can comprise steps for obtaining kinetic data. The kinetic data can include one or more rates of change. In certain embodiments, the kinetic data can be obtained under conditions of differential diffusion, for example, using a flush assay. In a flush assay, the fluorescence of unbound reporter molecules, RMSA complexes, and/or some combination thereof is detected while flushing the flow region of the microfluidic device with a medium that substantially lacks reporter molecules, secreted analyte of interest, and RMSA complexes. During the flush, molecules at higher concentrations within the chamber (e.g., reporter molecules, secreted analyte of interest, RMSA complexes) diffuse down their concentration gradients, moving from a region of relatively high concentration (e.g., an unswept region of the chamber) to a region of relatively low concentration (i.e., the flow region). As a result of differences in the size and mass of the reporter molecules as compared to the RMSA complexes, differential diffusion can take place, with the lower mass reporter molecules diffusing out of the chamber and into the flow region at a faster rate than the higher mass RMSA complexes. By detecting the reporter molecules and RMSA complexes at a plurality of time points after the start of the flush, the relative contributions of the reporter molecules and the RMSA complexes to the detected signal can be determined. In some embodiments, determining the relative contributions of the reporter molecules and the RMSA complexes comprises comparing the detected signal to signal detected in another chamber of the microfluidic device, where the other chamber lacks a biological micro-object (and thus a source of analyte of interest).

In some embodiments, detecting the reporter molecules and/or RMSA complexes in the methods for assaying an intrinsic diffusion gradient and/or assessing (e.g., quantifying) a level of secretion of an analyte of interest from a biological micro-object or a population of biological micro-objects (e.g., a clonal population) can comprise imaging a region of interest within the microfluidic device. The image(s) can be taken of the associated chamber(s) and flow region(s) in the microfluidic device. The image can then be analyzed, e.g., to calculate a score correlated with the level of secreted analyte of interest in the chamber(s). In some embodiments, one or more (e.g., a plurality of) images is taken, e.g., over a period of time, or across one or more fields of view, regions of interest, fluorescent channels, etc. In some embodiments, an image or plurality of images may be taken at a first period of time, and subsequently an image or plurality of images can be taken at a second period of time. In some instances, images are taken as the system is approaching the equilibrium state and/or after the system reaches steady state. The period of time required to reach equilibration (e.g., steady state equilibrium) can be up to about 3 hours or more (e.g., greater than about 10 minutes, greater than about 30 minutes, greater than about 1 hour, greater than about 1.5 hours, greater than about 2 hours, greater than about 2.5 hours, greater than about 3 hours, greater than about 3.5 hours, greater than about 4 hours, greater than about 4.5 hours, or longer). The images can be taken, for example, during continuous flow, during pulsed flow, or during stopped flow.

In some embodiments, the methods for assaying an intrinsic diffusion gradient and/or assessing (e.g., quantifying) a level of secretion of an analyte of interest from a biological micro-object or a population of biological micro-objects (e.g., a clonal population) can further comprise expanding the biological micro-object within the chamber into a clonal population (e.g., derived from a single cell) of biological micro-objects. Expanding the biological micro-object within the chamber can include flowing a culture medium through the flow region for a period of time.

Optical Calibration. Prior to any imaging, the optical system and microfluidic device may be configured using one or more methods of optical calibration. In certain embodiments, optical calibration can any method of optical alignment commonly known in the art. Optical alignment can comprise determining alignment of a filter cube, dichroic, or other optical component relative to a desired output or output range (e.g., power density at a particular location-including but not limited to the microfluidic device, a CCD camera, or another detector capable of calculating power density or a variable equivalent to or correlated with power density or another desirable output indicative of alignment of light moving through the optical train of the system, where the optical train is configured for operating and/or imaging the microfluidic device (e.g., microfluidic chip). In some instances, optical alignment can comprise aligning the focal plane and or objective of the optical train; such methods can include but are not limited to collecting one or more z dimension images and assessing the focus according to one or more features of the system configured for operating and/or imaging the microfluidic device.

In some embodiments, optical calibration can comprise applying methods for performing one or more image processing operations (flat fielding, normalization, masking, image subtraction, etc. on an image or set of images obtained from a microfluidic device. In any number of instances, a combination of the image processing operations (flat fielding, normalization, image subtraction, masking etc.) can be stacked together to yield a useful corrected image or corrected image set that can be used for further analysis. Methods for subtraction can comprise image subtraction and or pixel subtraction, whereby the digital numeric value of one pixel or a whole image is subtracted from another image. Methods of normalization can comprise taking a value of a given image (e.g., intensity value) and dividing it by an aggregated value (e.g., a global average intensity value). Methods for masking can comprise eliminating one or more sections of an image (e.g., sections where no signal should be present and/or where excessive background may interfere with, for example, calculation of a score correlated with the level of secreted analyte of interest in the chamber(s)).

In certain embodiments, the methods disclosed herein can comprise flat fielding. The term “flat fielding” as used herein refers to methods known it the art for improving the quality of the image relative to a result by eliminating artifacts (e.g., variations in pixel-to-pixel sensitivity of a detector, distortions of an optical path, etc.) by applying a flat field to compensate for variations in gains and dark currents across a detector such that a uniform signal detected by the detector can generate a uniform output. In some embodiments, the optical system and microfluidic device may be aligned across one or more axes (x, y, z), and, optionally, additional flat fielding may be applied. Such flat fielding can comprise, for example, applying a quadratic correction derived from the measurement of a uniform optical target. Flat fielding can be used in conjunction with any other image processing operations known in the art or combinations thereof.

Flat fielding and any other image processing operations or combination of image processing operations can be performed using one or more reference images. Examples of reference images include a Dark Reference image of the microfluidic device and a signal reference image. A dark reference image can be taken in the absence of one or more of: cells, medium, reporter molecule, etc., with the aim of providing dark reference values that can be used to correct for autofluorescence errors and other system errors at each pixel of the corrected image. A signal reference image, which can be taken of the microfluidic device with, for example, a measurable signal associated with a known quantity of signal producing component (e.g., fluorescent dye of a known concentration flowing through the microfluidic device) can be utilized with the aim of correcting for optical roll off, photobleaching errors, camera errors, etc. which have effects on the signal being measured.

In some embodiments, detecting the reporter molecules located within the area of interest further may include determining a background-subtracted signal intensity by subtracting an intensity of a background signal from the measured intensity of the detectable signal. The background signal may not be measured every time reporter molecules are detected. In some embodiments, the background signal may be pre-determined based on known/standard conditions (e.g., chip type, location of chamber in the chip, type of detectable label, components of first fluidic medium).

Methods disclosed herein may further include measuring an intensity of a background signal within the area of interest, at a time prior to introducing the biological micro-object into the chamber. In various embodiments, the measured intensity of the detectable signal or the background-subtracted signal intensity may be normalized for a number of cells observed within the chamber. The micro-objects may be measured using brightfield imaging, and counted using a cell counting method such as that disclosed International Publication No. WO 2018/102748.

In one or more embodiments, optical calibration can comprise calibration of the location of key features of a microfluidic device relative to the field of view or fields of view in images of the microfluidic device. In some instances, chip can include but is not limited to obtaining an image of a chip comprising one or more pattern(s) or feature(s) (e.g., a pattern or design etched, embedded or otherwise disposed) located at know locations on the microfluidic chip. Optical calibration of the microfluidic chip can comprise taking images of the pattern(s) or feature(s) and determining location of the microfluidic chip.

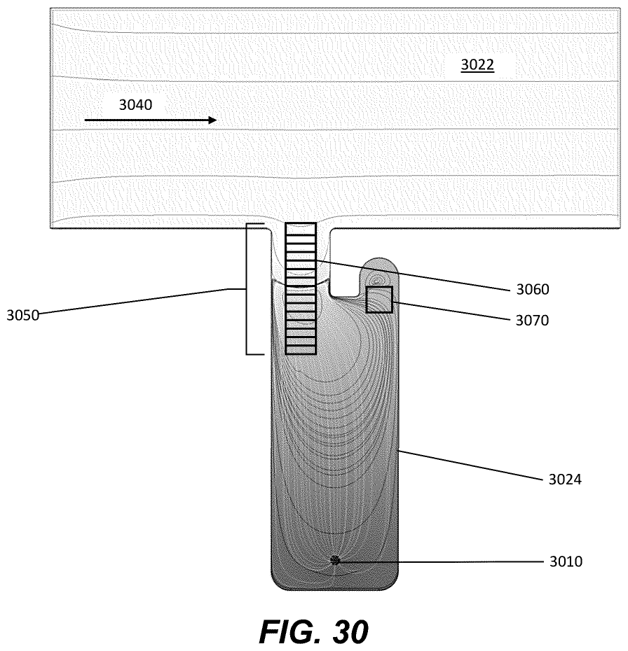

Region of Interest. In accordance with various embodiments, the region of interest may comprise at least a portion of the chamber (e.g., an upswept region of the chamber), as depicted in FIG. 30. In certain embodiments, the chamber 3024 may have an isolation region and a connection region fluidically connecting the isolation region to the flow region, wherein the isolation region and the connection region are configured such that components of a fluidic medium in the isolation region are exchanged with components of a fluidic medium 3040 in the flow region substantially only by diffusion. In such embodiments, the region of interest 3060, 3070 may comprise a portion of the isolation region, a portion of the connection region, a portion of the flow region 3022 immediately adjacent to the connection region, or any combination thereof.

In some embodiments, a region of interest 3060, 3070 can comprise a region located between the location of biological micro-object(s) 3010 within a chamber 3024 and the opening of the chamber to a flow region 3022 of the microfluidic device. In certain embodiments, the region of interest 3060 may include at least a portion of the chamber 3024 aligned along an axis of diffusion (e.g., 3050) from within the chamber 3024 to out into the flow region 3022. Thus, the region of interest 3060 can include one or more regions that lie along an axis of diffusion (e.g., 3050) within the system. In certain embodiments, an axis of diffusion (e.g., 3050) can comprise a portion of or the entirety of a connection region of the chamber, and, optionally, the axis of diffusion (e.g., 3050) can further comprise a portion of an isolation region and/or a flow region of a microfluidic device. In other instances, the region of interest 3070 can include one or more regions that lie off of an axis of diffusion (e.g., 3050) within the system. For example, the region of interest 3070 can include a portion or extension of the chamber 3024 (or an isolation region thereof), that extends away from the region where biological micro-object(s) 3010 would typically accumulate. Such a portion or extension can be a blind/dead-end extension, such as a hook region.

In certain embodiments, the region of interest may be located in a region of the chamber that lacks biological micro-objects and/or is less sensitive to the location of the biological micro-object(s) within the chamber. In certain related embodiments, the region of interest may be located at least 1 micron, 2 microns, 3 microns, 4 microns, 5 microns, 6 microns, 7 microns, 8 microns, 9 microns or 10 microns from the biological micro-object (e.g., cell) within the chamber. In certain embodiments, the region of interest can be located in a region of the chamber that is less sensitive to artificial background signal generated by, e.g., the edges of the chamber. Thus, in certain embodiments, the region of interest will be located at least 1 micron, 2 microns, 3 microns, 4 microns, 5 microns, 6 microns, 7 microns, 8 microns, 9 microns or 10 microns away from the edge or wall the chamber. In certain embodiments, the region of interest (or a sub-region thereof) can include a dimension (e.g., a width or a length) of at least about 10 microns (e.g., at least about 15 microns, at least about 20 microns, about least about 25 microns, or more). In certain embodiments, the region of interest (or a region thereof) can include an area of at least about 100 square microns (e.g., at least about 150, 200, 250, 300, 350, 400, 450, 500, 550, 600, 650, 700, or more square microns). More generally, the size of the region of interest may be as small as the smallest unit of resolution of the detection device (e.g., as small as a single pixel), but there is a trade-off with reducing the size of the region of interest because the signal:noise ratio generally goes down as the size of the region of interest is reduced. Moreover, as the size of the region of interest increases, the likelihood that the region of interest encompasses regions of the microfluidic device that may contain signaling artifacts (e.g., regions that include biological micro-objects) or signal arising from adjacent chambers also increases. Accordingly, regions of interest having an intermediate size (e.g., about 100 square microns to about 5,000 square microns, or about 200 square microns to about 1000 square microns, about 300 square microns to about 700 square microns, about 500 square microns to about 1500 square microns, about 1000 square microns to about 4000 square microns, about 2000 square microns to about 4000 square microns, about 3000 square microns to about 4000 square microns, or any range formed by two of the foregoing end points) can be advantageous for various embodiments of the methods of assaying an intrinsic diffusion gradient and/or methods of assessing or quantifying a level of secretion of an analyte of interest disclosed herein.

In certain embodiments, the region of interest may be divided into sub-regions. See, e.g., FIG. 30 & element 3060. A region of interest (e.g., 3060) may have subregions of differing sizes (e.g., pixel size) or each subregion may have the same size (e.g., pixel size). In some embodiments, a region of interest (e.g., 3060) can comprise a plurality of subregions, wherein said plurality is from about 2 to about 50 (e.g., about 3 to about 35, about 4 to about 25, about 5 to about 15, or any range defined by two of the foregoing endpoints). In some embodiments, a most distal subregion of a region of interest (e.g., 3060) is located furthest away from the flow region (or channel) 3022 but is selected such that it does not overlap with the biological micro-object(s) 3010, and the most proximal subregion of the set of subregions of the region of interest (e.g., 3060) is located closest to or within the flow region (or channel) 3022. The plurality of sub-regions from the most proximal subregion to the most distal subregion may be located at a region from which the detected signal is used to assess the relative or absolute amount of a secreted analyte of a biological micro-object within a chamber (e.g., 3024). In certain embodiments, a region of interest that includes a plurality of sub-regions can include an aggregate area of at least about 500 square microns (e.g., at least about 750, 1000, 1500, 2000, 2500, 3000, 3500, 4000, 4500, 5000, 5500, 6000, 6500, or more square microns).

In certain embodiments, the region of interest, or subregions thereof, can be used to quantify imaging data, onto which a variety of mathematical operations may be performed to extract information about the relative or absolute amount of the secreted analyte. Such operations can include calculation of median, mean, and, when using a set of subregions, one or more values such as a slope indicative of an average intensity change across two or more subregion from the region of interest or integration of total intensity across two or more subregions.

Imaging data. Imaging data (e.g., an image, series of images, etc.) can comprise one of or a combination of background image(s), signal reference (e.g., fluorescence reference) image(s), diffusion reference image(s), and assay image(s).

A background image can be taken by an imaging device prior to any foreign matter (such as, for example, micro-objects, reporter molecules, or other reagents) being introduced into the microfluidic device. In so doing, the background image captures any background noise in the device, particularly in regions of interest. Background noise can be due to, for example, artifacts, or instrument setup and imaging parameters, including but not limited to light from the excitation source, camera noise, and ambient light. Background noise can also be due to background signal (e.g., fluorescence) imparted by, for example, auto-fluorescence of samples, vessels, imaging media, or the fluorescence resulting from fluorophores not bound to specific targets. The image area included in the background image may depend on how the image is implemented on the system going forward. For example, as will be described in detail below, depending on the calibration methods used, a different background image area may be desired.

A signal reference image can be taken by an imaging device after a reporter molecule is introduced into the flow region of the microfluidic device and the reporter molecule concentration equilibrates between the flow region and chambers of the microfluidic device, including in any regions of interest. In so doing, the signal reference image captures image acquisition distortions in the device and system. Such distortions can stem from, for example, microfluidic device and/or imaging element design. Image distortion types can include, for example, image edge effects, perspective distortion, barrel distortion, pincushion distortion, mustache distortion, and chromatic aberration. The signal reference image area can include an image of the region of interest, the flow region proximate the chamber and associated region of interest, or both. The image area included in the signal reference image can depend upon how that image is implemented by the system going forward. For example, as provided in further detail below, depending on the calibration methods implemented by the system, a different signal reference image area may be utilized.

In some instances, a diffusion reference image can be taken following introduction of a fluidic medium free of reporter molecules, where the fluidic medium free of the binding agent is perfused after the fluidic medium comprising the binding agent is introduced into the microfluidic device. In such instances, the signal reference image may be referred to as a “diffusion reference image.” The diffusion reference image may comprise a series of images taken over time of one or more fields of view. Analysis of the diffusion reference image can comprise measuring a change in signal as a function of time, determining a correction value or a slope from which correction values can be derived, and applying the correction value comparison of images to a single reference timepoint.

Normalization of the Assay image. Before the Assay Image can be processed to assess relative or absolute amounts of a secreted analyte, the raw Assay Image may be normalized. In some embodiment, the raw Assay Image may be normalized by subtracting both a Dark Reference image and a Signal Reference image correction from each pixel in the raw Assay Image as in the following equation:

$$Normalized Assay value = \frac{Assay Intensity value - Dark Reference value}{Signal Intensity value - Dark Reference value}$$

The Dark Reference image may be obtained by imaging the microfluidic device before introducing the biological micro-object. Autofluorescence errors and other system errors can be corrected by subtracting the Dark Reference value at each pixel. The Signal Reference Image may correct for roll off, vignetting, and other optical artifacts. The Signal Reference Image can be obtained by flowing reporter molecule (e.g., just the reporter molecule label) throughout the microfluidic device to reach an equilibrated concentration of the reporter molecule or label. Each pixel in the raw Assay Image may be corrected in this manner, before extracting the signal data for quantitation purposes. In some embodiments, a smoothing algorithm may be further applied to reduce noise.

Quantification of the assay signal. In some embodiments, the diffusion profile of the RMSA may be used to quantify the amount of the RMSA complex present in the chamber. The diffusion profile provides a series of values (e.g., signal intensity values) that represent the concentration of the RMSA complex as it diffuses from its source to the channel. After identification of the region of interest, other transformations may be applied. For example, the pixels in each line may be processing by discarding outlier and/or aberrant pixels, other forms of global/local normalization, space conversion, and transforming the space of the pixel (e.g. from a multi-dimensional space to a two-dimensional space or vice-versa).

Depending on the embodiment, the signal intensity values may be used in different ways to calculate values that are proportional to concentration values. In some embodiments, the AOI may be sampled at fixed points to generate a set of concentration values corresponding to the signal intensity values at the fixed points. In some embodiments, the region of interest may be segmented into a series of subregion and the median or mean intensity of each subregion may be calculated. Based on the embodiment and the degree of resolution required, the number of concentration values calculated can be as low as 1 and as high as the number of pixels representing the diffusion trajectory.

Depending on the embodiment, the concentration values may be combined in different ways in order to quantify the amount of signal from the bound reporter molecules (and therefore an amount of secreted analyte of interest) present. In some embodiments, the concentration values may be plotted to assess whether concentration values exhibit characteristics consistent with a diffusion profile. Depending on the embodiment, a number of algorithms may be used to fit a line to the concentration values and calculate characteristics of the line such as the slope and error associated with the line. Suitable line-fitting algorithms include: least-squares, polynomial fit, curve-fitting, and erfc fitting. Other suitable algorithms are known to those skilled in the art.

### II. MICROFLUIDIC PREDICTION OF PRODUCTIVITY OF BIOMOLECULES FROM CELLS

The industrial synthetic biology sector has made huge investments to achieve relevant miniaturized screening systems for scalable fermentation or production. Over the past several decades, metabolic engineering has developed cell factories as sustainable alternatives to chemical synthesis from petroleum feedstocks or harvesting of animals and plants. These production processes employ natural and engineered enzymes to give elegant one-pot alternatives to conventional manufacturing. The current method most often employed for selection of a cell strain for manufacturing is the lab-scale bioreactor, which represents a costly and labor-intensive commitment, as thousands or more candidate genotypes are often considered. Most high-throughput strain improvement campaigns have heavily relied on hit selection from microplate-based culture models, growth-coupled selection schemes, and more recently droplet microfluidics, with varying success in the prediction of scalable bioreactor phenotypes.

A shortcoming of microplate- and droplet-based culture models is that they often do not replicate the conditions observed in feedback-regulated bioreactor systems. It is not feasible to individually and dynamically control the chemical composition of thousands of wells or droplets in a typical screening scheme, as is done in low-throughput bioreactors. Applicant has developed methods of culturing and assaying within a microfluidic device to addresses this challenge by spatially isolating strains in incubation chambers (NanoPen™ chambers) within the microfluidic device, the OptoSelect™ chip, while continuously controlling the extracellular environment by perfusion of fresh media. This approach has the potential to closely mimic a bioreactor by tightly controlling nutrient levels, oxygenation, and PH balance during growth.

Previous reports of similar “microchemostat” systems have been made for aspects of microbial culture and in situ assay, but these approaches have not demonstrated the throughput, automation, robustness, and operational feasibility that is desirable and/or are not intended to screen for improved metabolic flux. Accordingly, Applicant has discovered high-throughput (>10³ genotypes/week), chemostat-like screening methods to improve small-molecule secretion from microbial strains.

In the bioproduction industry, one severe problem is the expense, time and difficulty in identifying clonal populations having desired levels of production and growth habits when employing the currently available instrumentation and workflows. The ability to screen and identify promising clones within a microfluidic device, very early in expanding populations, such as 3, 4, 5, 6, or 7 days after seeding individual founding cells, as described herein, can offer significant time and cost advantages. However, culturing and screening cells for the bioproductivities in a microfluidic environment imposes technical problems, especially when the cells are relatively small or highly mobile. Those cells can be difficult to be contained in a chamber during culturing. Moreover, detecting of the secreted product can be challenging when the cells have a lower secretion level, which impose uncertainties of whether the secreted products at early stage of culture are of sufficient concentration for detection and screening.

Methods for evaluating bioproductivity of a cell. One aspect of the present disclosure is shown in a workflow for evaluating bioproductivity of a cell, as shown in FIG. 6. The workflow uses a microfluidic device (i.e. a chip) as described herein and includes: preparing the microfluidic device for the workflow 605, introducing and penning cells into respective chambers 610, forming an in situ-generated barrier within the chamber 615, culturing cells in the chamber, where in some embodiments, culturing produces a clonal population 620, optionally measuring biomass of the cells in each chamber 640, performing an assay on-chip 645, and exporting the cells may be included 650. The assay can be an accumulation assay, a diffusion gradient assay, or a bead assay as described herein. In the embodiments where a bead assay is performed, a step of loading an assay bead 605 is conducted after the culturing process has been performed 620. Further, when induction of secretion of the product is needed, induction 635 may be performed after introduction of the bead 625. In some variations, when induction 635 is included in the workflow using a bead assay, a further process is inserted, where after loading the bead for the bead assay 625, an additional period of recovery, e.g., a process including another period of culturing 630 is added before instituting the process of induction 635. Induction 635 is performed before assay 645. In some embodiments, biomass measurement 640 is also performed before assaying 645 is performed. In other embodiments, an induction process 635 is not required, depending on the nature of the engineered cell under examination. In such cases, after introduction of a bead 625, the overall workflow will not include either the process of recovery 630 or induction 635.

In embodiments employing either an accumulation assay or a diffusion assay, the workflow method will include chip prep processes 605, penning cells process 610, hydrogel barrier introduction process 615, cell culture process 620, and assay process 645. In some embodiments, the process of exporting cells may be included after assay process 645. In some variations, induction of secretion of the analyte produced by the cells 635 will be included after the process of culturing (Box 620) and before assaying 645. Further, biomass measurement process may be included before assaying, and after induction. A recovery process 630 may not be included as bead importation is not included in these variations. In other variations, induction of secretion of the analyte is not needed, and process 635 is not included. However, biomass measurement may be included in this further variation.

Other workflows described herein, such as assays including a sealed chamber assay or an open chamber assay omit the process of introducing a hydrogel barrier. In these variations, any of the processes shown in 605, 610, 620, 635, 640, 645, and 650 may be included and combined to form a complete workflow.

Methods are provided for evaluating bioproductivity of a cell. The methods includes: disposing a cell, which may be a non-mammalian cell into a chamber of a microfluidic device, the microfluidic device having a microfluidic circuit comprising a flow region and the chamber, wherein the chamber comprises an opening to the flow region; forming an in situ-generated barrier within the chamber, wherein the in situ-generated barrier defines an enclosed culture area within the chamber, e.g., subdivided therein, for culturing the cell; allowing the cell to secrete an analyte within the enclosed culture area; introducing a first fluidic medium comprising a reporter molecule into the flow region of the microfluidic circuit, wherein the reporter molecule is configured to bind to the analyte to form a reporter molecule: secreted analyte complex (RMSA complex), wherein the reporter molecule comprises a first detectable label; and detecting a first signal associated with the first detectable label within an area of interest within the microfluidic circuit, thereby evaluating the bioproductivity of the cell.

Bioproductivity. As used herein, “bioproductivity” refers to productivity of a living cell in producing or secreting a molecule of interest. The term “bioproductivity” and “productivity” are interchangeable in this description. As used herein, “molecule of interest,” “biomolecule of interest,” “biomolecule,” “analyte,” “secreted analyte,” “secreted protein,” and alike are interchangeable and refer to a biomolecule or an organic molecule produced by a cell of which the bioproductivity is to be evaluated. In some embodiments, the analyte is an amino acid, a polypeptide, a protein, a nucleotide, a nucleic acid, a polysaccharide, or a combination thereof.

In some embodiments, the biomolecule may be a small organic molecule having a molecular weight less than about 100, 90, 80, 60, 50, 40, 30, 20, 10 kDa. In some embodiments, the biomolecule may be a small organic molecule having a molecular weight less than about 2000, 1500, 1200, 1000 Da.

Secreted Analyte. In various embodiments, the analyte secreted by the cell, e.g., bioproduct, may include a protein, a saccharide, a nucleic acid, an organic molecule other than a protein, saccharide, or nucleic acid. A secreted analyte (e.g., analyte) can diffuse in the media, and can comprise a broad range of molecular weights. In various embodiments, the analyte secreted by the biological micro-object may be a protein. The secreted analyte can comprise a molecular weight, wherein said molecular weight is proportional to a diffusion rate and therefore correlated with how much (e.g., the concentration) of the secreted analyte that accumulates in the chamber under a steady state equilibrium.

A secreted analyte may be a naturally expressed analyte (e.g., natively expressed) or may be a bioengineered analyte (e.g., a product resulting from gene insertion, deletion, modification and the like). A secreted analyte that is a nucleic acid may be a ribonucleic or a deoxynucleic acid, may include natural or unnatural nucleotides. A secreted analyte that is a saccharide may be a mono-, di- or polysaccharide. Non-limiting examples of saccharides may include glucose, trehalose, mannose, arabinose, fructose, ribose, xanthan or chitosan. A secreted small, organic molecule may include but is not limited to biofuels, oils, polymers, or pharmaceutics such as macrolide antibiotics. A secreted analyte that is a protein can be an antibody or fragment of an antibody. A secreted analyte that is a protein can be a blood protein, such as an albumin, a globulin (e.g., alpha2-macroglobulin, gamma globulin, beta-2 microglobulin, haptoglobulin), a complement protein (e.g., component 3 or 4), transferrin, prothrombin, alpha 1 antitrypsin, and the like; a hormone, such as insulin, glucagon, somatostatin, growth hormone, growth factors (e.g., FGF, HGF, NGF, EGF, PDGF, TGF, Erythropoietin, IGF, TNF), follicle stimulating hormone, luteinizing hormone, leptin, and the like; a fibrous protein, such as a silk or an extracellular matrix protein (e.g., a fibronectin, laminin, collagen, elastin, vitronectin, tenascin, versican, bone sialoprotein); an enzyme, such as a metalloprotease (e.g., matrix metalloproteinase (MMP)) or other types of protease (e.g., serine protease, cysteine protease, threonine protease, aspartic protease, glutamic protease, asparagine peptide lyase), an amylase, a cellulase, a catalase, a pectinase, and the like; a bacterial, yeast, or protozoan protein; a plant protein; or a viral protein, such as a capsid or envelope protein. A secreted analyte that is a protein can be an enzyme (including but not limited to a proteolytic enzyme), an engineered (normally intracellular protein) protein, such as for example, albumin, and/or a structural protein including but not limited to silkworm silk or spider silk). This list is not limiting and any protein that may be engineered to be secreted may be evaluated by the methods. The secreted analyte may be an antibody-drug conjugate. A non-limiting example of a secreted analyte that may have a combination of a protein, a saccharide, a nucleic acid, an organic molecule having a molecular weight of less than 3 kDa, and/or a virus, can include a proteoglycan or glycoprotein. The secreted analyte may include an engineered binding site commonly used for purification. Said purification tags can include but not limited to be a structured or unstructured binding domain configured to associate with a reporter molecule.

Penning cells. In some embodiments, disposing a cell into a chamber of a microfluidic device comprises: obtaining a microfluidic device comprising a microfluidic circuit comprising a flow region and a chamber fluidically connected to the flow region; introducing a fluidic medium comprising the cell into the flow region; and disposing the cell into the chamber. In some embodiments, the cell is disposed into a chamber of a microfluidic device by gravity or by OEP as described herein. In some embodiments, positive OEP or negative OEP can be selected depending on the surface charge of the cell to be moved. For example, mammalian cells are generally negatively charged at physiological pH so that a negative OEP can be applied to move the cells by compelling them toward a direction. In other examples, cells such as yeast cells are typically positively charged so that a positive OEP will be suitable for moving the cells.

In some embodiments, when the cell to be penned is small (e.g., nonmammalian cells, which tend to be smaller than mammalian cells), for example, smaller than 10 microns in diameter, the OEP is performed at a higher voltage. In some embodiments, the voltage is higher than 5, 6, 7, 8, 9, 10, 11, 12, 13, 14, or 15V. In some embodiments, the diameter of the cell is about 0.5, 1, 1.5, 2, 2.5, 3, 3.5, 4, 4.5, 5, 5.5, 6, 6.5, 7, 7.5, 8, 8.5, 9, 9.5, 10 microns.

The process of penning cells may be automated using image recognition software as described in U.S. Application Publication No. US2019/0384963, filed on May 31, 2019, and U.S. application Ser. No. 17/103,414, filed on Nov. 24, 2020, each of which disclosures is herein incorporated in its entirety by reference. In various embodiments, the cells are moved into separate chambers, e.g., NanoPens, to be cultured as individual colonies. By penning a single cell to an individual chamber, expansion to a population of cells provides a clonal population. The ability to observe, test, and export selectively a specific clonal population of cells that exhibit desired characteristics, provided by the methods described herein, is an important improvement over the macroscale techniques currently used for developing engineered cell lines that can produce a desired bioproduct. To more effectively harness the potential for assay processes carried out within each of many individual chambers within the microfluidic device, barriers can be introduced into the chamber for a number of purposes that may improve the assay by enhancing the accuracy, sensitivity, or reproducibility, amongst other properties, of the assay. For example, a barrier may introduce, e.g., generated in situ, in order to sequester cells away from the assay observation area (e.g., assay area or area of interest) so a rapidly secreting cell does not artificially enhance the detected signal for the entire chamber by being present within an area of interest as a point source. Barriers may also be introduced to prevent a secreted analyte that is bound to a reporter molecule (RMSA complex) from diffusing away from an area of interest. Barriers also can prevent molecules having a size (molecular weight) too large to pass through the barrier to reach an area of interest of an assay which might interfere with the assay mechanism.

In situ-generated barrier. Generally, one of the functions of the in situ-generated barrier in the methods of the present disclosure is to contain a cell within the chamber of the microfluidic device. The term “in situ-generated barrier” refers to a barrier that is formed in a selected area while the microfluidic device is in operation. The barrier is generally not formed while manufacturing the microfluidic device or does not exist before the microfluidic device is used for experiments or research. The term “barrier” refers to a physical structure that is formed and fixed, at least for a certain period of time, in a selected area and is capable of impeding or blocking a particle from crossing through the barrier. As a result, the barrier formed in situ within the chamber can separate the inner space thereof into two areas on each side of the barrier. In some embodiments, the barrier defines an enclosed culture area within the chamber. In some embodiments, the barrier defines within the chamber an assay area and an enclosed culture area.

As used herein, “enclosed culture area” refers to an area pre-determined for maintaining or culturing a cell, but the methods of the present disclosure are not limited to maintain or culture the cell in the enclosed culture area. The term “enclosed” describes that the culture area is substantially closed so that the cell cultured cannot easily move or be moved out of the area. However, the term “enclosed” is not limited to require that the area is completely closed or sealed. Some substances can still move in and out of the area (for instance, the culture medium, containing nutrients and/or waste can diffuse in and out of the area). Furthermore, the cell cultured in the culture area can still move or be moved in and out of the area in some variations. In some embodiments, a specifically created opening exists to allows substance (including cells) to enter or leave the enclosed culture area. In certain embodiments, the “opening” is a space between the in situ-generated barrier and one or more surfaces of the chamber, wherein the space is at least 2.0×, 2.5×, 3.0×, 3.5×, 4.0×, 4.5×, 5.0× or greater, or any range defined by two of the foregoing endpoints, where x is the average diameter of the cell.

As used herein, “assay area” refers to an area pre-determined for performing an assay required by the methods of the present disclosure. However, it is not limited that an assay required by the method of the present disclosure can only be performed within that array. It is also not limited that any areas of the chamber other than the assay area cannot be used for performing an assay.

In some embodiments, the impediment or block produced by introduction of the barrier is size-dependent. A particle can be impeded, blocked, or allowed to cross through the barrier depending upon its size. In some embodiments, the in situ-generated barrier has a porosity that substantially prevents a cell from crossing through the in situ-generated barrier. In some embodiments, the in situ-generated barrier can comprise a gap having a width or diameter that allows a cell to pass through the barrier. Nevertheless, the movement of the cell through the barrier via the gap can still be impeded.

In another aspect, a hydrogel barrier may be additionally used to reduce clonality risk. As described below, a second hydrogel barrier may be introduced, for example, after completion of assay and identification of desired cells or clonal populations within a chamber. In order to prevent loss of clonality as the desired cells are exported from the chamber, and optionally from the microfluidic device, a uniform hydrogel barrier, as described herein, may be formed across the width of the openings of undesired/unselected chambers, reducing the risk that cells that do not belong to the selected clonal population will be unpenned and mix with the cells selected for export.

Hydrogel in situ-generated barrier. In certain embodiments, the in situ-generated barrier is a hydrogel. In certain embodiments, the in situ-generated barrier comprises a solidified polymer network. In some embodiments, the solidified polymer network comprises a synthetic polymer, a modified synthetic polymer, or a biological polymer. In certain embodiments, the solidified polymer network comprises at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. In some embodiments, the solidified polymer network does not include a silicone polymer.

Physical and chemical characteristics determining suitability of a polymer for use in the solidified polymer network may include molecular weight, hydrophobicity, solubility, rate of diffusion, viscosity (e.g., of the medium), excitation and/or emission range (e.g., of fluorescent reagents immobilized therein), known background fluorescence, characteristics influencing polymerization, and pore size of a solidified polymer network. The solidified polymer network is formed upon polymerization or thermal gelling of a flowable polymer solution containing at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. Various co-polymer classes may be used, including but not limited to any of the above listed polymers, or biological polymers such as fibronectin, collagen or laminin. Polysaccharides such as dextran or modified collagens may be used. The flowable polymer may be referred alternatively here as a pre-polymer, in the sense that the flowable polymer is crosslinked in-situ. Biological polymers having photoactivatable functionalities for polymerization may also be used.

In some instances, a polymer may include a cleavage motif. A cleavage motif may include a peptide sequence inserted into the polymer that is a substrate for one or more proteases, including but not limited to a matrix metalloproteinase, a collagenase, or a serine proteinase such as Proteinase K. Another category of cleavage motif may include a photocleavable motif such as a nitrobenzyl photocleavable linker which may be inserted into selected locations of the prepolymer. In some embodiments, a nitrobenzyl photocleavable linker may include a 1-methinyl, 2-nitrobenzyl moiety configured to be photocleavable. In other embodiments, the photocleavable linker may include a benzoin moiety, a 1, 3 nitrophenolyl moiety, a coumarin-4-ylmethyl moiety or a 1-hydroxy 2-cinnamoyl moiety. A cleavage motif may be utilized to remove the solidified polymer network of an isolation structure. In other embodiments, the polymer may include cell recognition motifs including but not limited to a RGD peptide motif, which is recognized by integrins.

One type of polymer, amongst the many polymers that may be used, is polyethylene glycol diacrylate (PEGDA) or polyethylene glycol acrylamide (diacrylamide, multi-armed acrylamide or substituted versions as described herein).

Photoactivated polymerization may be accomplished using a free radical initiator Igracure® 2959 (BASF), a highly efficient, non-yellowing radical, alpha hydroxy ketone photoinitiator, is typically used for initiation at wavelengths in the UV region (e.g., 365 nm), but other initiators may be used. An example of another useful photoinitiator class for polymerization reactions is the group of lithium acyl phosphinate salts, of which lithium phenyl 2, 4, 6,-trimethylbenzolylphosphinate has particular utility due to its more efficient absorption at longer wavelengths (e.g., 405 nm) than that of the alpha hydroxy ketone class. Another initiator that may be used are water soluble azo initiators, such as 2, 2/Azobis [2-methyl-N-(2-hydroxyethyl)propionamide]. The initiator may be present within the flowable polymer solution at a concentration of about 5 millimolar, about 8 millimolar, about 10 millimolar, about 12 millimolar, about 15 millimolar, about 18 millimolar, about 20 millimolar, about 22 millimolar, about 25 millimolar, about 28 millimolar, about 30 millimolar, about 35 millimolar, or about 40 millimolar.

Crosslinking may be performed by photopatterning of linear or branched PEG polymers, free radical polymerization of PEG acrylates or PEG acrylamides, and specifically tailored chemical reactions such as Michael addition, condensation, Click chemistry, native chemical ligation and/or enzymatic reactions. In particular, photopatterning of crosslinking may be used to gain precise control of extent of the physical extent of the hydrogel barrier as well as the degree of crosslinking, as described in the following section and in the Examples.

Inhibitors may be included within the flowable polymer solution to ensure precise control of photopatterning and to prevent extraneous or undesired polymerization. One useful inhibitor is hydroquinone monomethyl ether, MEHQ, but other suitable inhibitors may be used. The inhibitor may be present in the flowable polymer solution at a concentration of about 1 millimolar, about 2 millimolar, about 5 millimolar, about 10 millimolar, about 15 millimolar, about 20 millimolar, about 25 millimolar, about 30 millimolar, about 35 millimolar, about 40 millimolar or more, as needed to provide the photopatterning control desired.

Tuneable permeability. One aspect of performing assays using a hydrogel in an in situ-generated barrier is to determine what species is desired to gain access to the area of interest. Selection of the chemical nature of the polymer (for example, molecular weight range, number of cross linkable moieties per polymer unit (linear, 2 arm, 4 arm, 8 arm, star or comb polymer), mixtures of polymers), the amount of initiator, and mode of polymerization are variables that may be modified to tune the hydrogel barrier formed. Generally, the initiator is a photoinitiator. Photopatterning provides precise control of the geometry of the polymerization as well as the extent of polymerization, and changes in exposure time and power of the illumination also can provide more control to arrive at a desired type of porosity and degree of robustness of the polymerized feature.

As described in Example 2-1, non-limiting examples of controlled permeability are shown. Mixtures of two different flowable polymers having similar molecular weight were found to be advantageous in providing hydrogel barriers with differential permeability. Using polymers having similar molecular weights confers similar rates of diffusion, which simplifies delivery to the region within the chamber. Since chambers that are sequestration pens are unswept region of the microfluidic device, introduction of the polymer into the sequestration pen occurs substantially only by diffusion.

In many variations, polymer selection may depend upon the biocompatibility of the polymer species, and may be related to the specific application to which a hydrogel in situ-generated barrier may be used.

In some variations, the hydrogel may be a polyethylene glycol polymer or a modified polyethylene glycol polymer.

A wide range of molecular weights of the flowable polymer may be suitable, depending upon the structure of the polymer. In some embodiments, the flowable polymer may have a molecular weight of about 500 Da to about 20 kDa, or about 500 Da, about 1 kDa, about 3 kDa, about 5 kDa, about 10 kDa, about 12k Da, about 15 kDa, about 20 kDa or any value therebetween. A useful star type polymer may have Mw (weight average molecular weight) in a range from about 500 Da to about 20 kDa (e.g., four arm polymer), or up to about 5 kDa for each arm, or any value therebetween. In some embodiments, a polymer having a higher molecular weight range, may be used at lower concentrations in the flowable polymer, and still provide an in situ-generated barrier or isolation structure that may be used in the methods described herein.

Reversing/removing/minimizing the in situ-generated isolation structure. A number of mechanisms may be used to remove or reduce the in situ-generated hydrogel barrier when there is no further purpose for it. For example, once an assay is completed and desirable biological cells have been identified, it may be useful to remove the hydrogel barrier in order to export the cells and/or continue culturing and expanding the biological cell demonstrating desirable activities or properties.

Mechanical force. Increasing flow can be used if at least a portion of the hydrogel barrier is located within a flow region as opposed to an isolation region of a pen. For example, the at least one isolation structure may be located within an isolation region of a sequestration pen, and after the assay is complete, the sequestration pen or the isolation region therein may be modified to bring flow through the isolation region.

In other variations, such as is described herein, laser initiated bubbles may provide forces that can deform or disrupt the hydrogel barrier, permitting export of the cells, as is more fully described in Example 2-5.

Hydrolytic susceptibility: Porogens, including polymers which are incapable of being chemically linked to the photoinitiated polymer(s), may be included when forming the hydrogel barrier. The degree/size of openings within the formed hydrogel can customize the hydrolysis rate via accessibility within the hydrogel barrier). In other embodiments, the pores formed may be employed to permit secreted materials or chemical reagents to pass through the hydrogel barrier but prevent a cell from moving into, out of, and/or through the isolation structure. In other embodiments, degradability of these polymers may be increased by introducing degradable segments such as polyester, acetal, fumarate, poly(propylene fumarate) or polyhydroxyacids into polymers (e.g., PEG polymers).

Reducing agents: PEG may be formed with disulfide linkages at intervals along the macromere, which may be random or predetermined. The disulfide bonds may be broken by Dithiothreitol (DTT), mercaptoethanol, or TCEP.

Thermal: poly N-isopropylacrylamide (PNIPAm) or other suitable LCST polymers may be used to introduce hydrogel barriers upon heating. They may be removed by decreasing the temperature of the formed polymer hydrogel barrier. The polymers may include ELPs or other motifs that also permit removal by other mechanisms such as hydrolysis or proteolysis. In particular, PNIPAm may be used to create a surface for adherent cells, but then switched to permit export.

Proteolytic susceptibility: Hydrogels may have any sort of peptide sequence engineered in, such that selective proteolysis upon a selected motif by a selected protease can remove/reverse/or minimize a hydrogel isolation structure. Some classes of modified PEG include PEG having elastin like peptide (ELP) motifs and/or having peptide motifs for susceptibility to a variety of proteases (enzyme sensitive peptide ESP). A large number of these motifs are known. One useful motif is RGD which may be constrained to be cyclic.

Osmotic susceptibility: Calcium concentration/other osmotic strategies can be employed to degrade and remove a hydrogel barrier. As above, changes of media flowed through the channel or flow region may dimensionally swell or de-swell hydrogel barriers.

Photocleavage: As described above, if a polymer of the solidified polymer network includes a photocleavable moiety, directing illumination of an exciting wavelength to the solidified polymer network will cause cleavage within sections of the solidified polymer network. This cleavage may provide complete or partial disruption of the solidified polymer network, thereby removing or reducing the hydrogel barrier.

In some applications, the hydrogel barrier may not be removed but may simply be swelled or de-swelled using light or medialsolvent changes. Some types of hydrogels may incorporate moieties that respond reversibly to light (for example, change regiochemistry about a rigid bond; form reversible crosslinks within the polymer, or form/break ion pairs).

Cell exportation and geometry of the in situ-generated barrier. The in situ-generated barrier of the methods of the present disclosure provides advantages of containing a cell within the chamber and accumulation effects for assays. In some embodiments, the methods of the present disclosure further comprise exporting a cell from the chamber. In some embodiments, the cell is further exported out of the microfluidic device. In some embodiments, exporting the cell comprises directing a laser illumination upon a selected area of the chamber, as mentioned above, to create a bubble pushing the cell toward the opening of the chamber. In other embodiments, exporting the cell comprises directing laser illumination upon a selected area of the chamber to create a bubble dislodging the cell and dielectrophoretic forces may be used to move the cell once it is dislodged from the surface of the culturing area.

Without intending to be limited by theory, the laser illumination is set to project on a thermal target on a surface of the chamber to generate heating in the fluidic medium surrounding the thermal target may nucleate and propagate bubbles. The bubble, upon collapse, creates a cavitating force. In other embodiments, the bubble may be grown by continued illumination to create a shear flow of fluid directed towards nearby substances. The force and/or the flow can push the in situ-generated barrier, fluidic medium, and the cell away, typically in a direction towards the opening of the chamber. As a result, once the in situ-generated barrier no long holds its original position containing the cell, the cell can be moved out of the chamber. In some embodiments, the cell is moved by the bubble to a location close to an opening of the chamber to the microfluidic channel, and a OEP force is applied to further move the cell into the microfluidic channel where later the cell is flushed out of the microfluidic device by a flow introduced therein. In some other embodiments, once the laser pulse has been stopped the induced bubble collapses resultingly drawing fluid back towards the distal end of the chamber.

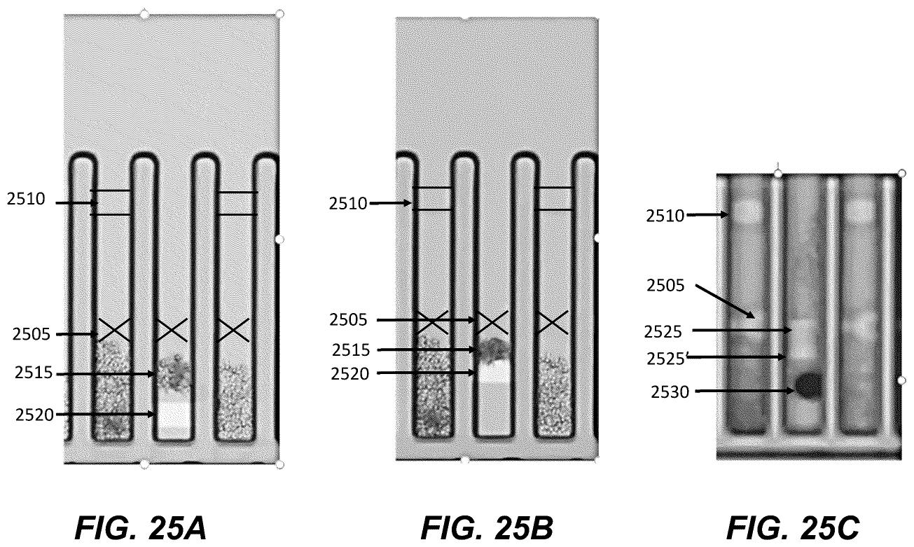

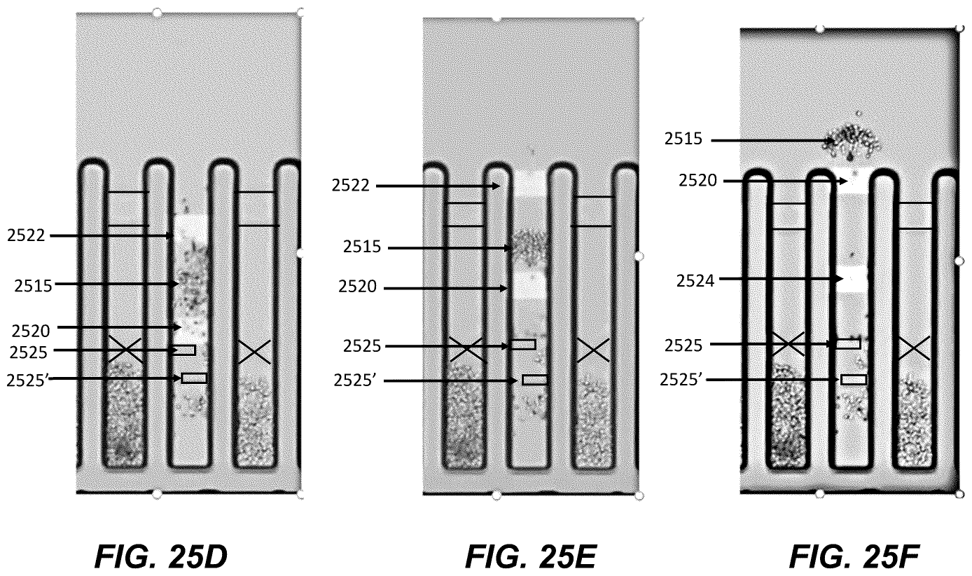

The site of illumination may be selected to be any discrete selected region of the microfluidic device, as may be useful. In some embodiments, the discrete selected region of illumination may be a location within a chamber (e.g., a sequestration pen) of a microfluidic device. In various embodiments, the discrete selected region of illumination is located within an isolation region of a sequestration pen, which may be configured like any sequestration pen described herein. In some embodiments, the laser illumination is projected at a cell-free area of the chamber. In some embodiments, the laser illumination is projected at an area near the distal end of the chamber. In certain embodiments, an OEP force is applied first to move the cell cultured in the chamber away from the distal end thereof to create a cell-free area, and then a laser illumination can be applied at the cell-free area to create a bubble. One method of exporting cells using laser illumination in the presence of a hydrogel barrier is further discussed below, and is shown in FIGS. 25A-25F.

The optical illumination, power of the laser, and other information regarding bubble dislodgement have been described, for example, in U.S. Pat. No. 10,829,728 (issued on Nov. 10, 2020) and U.S. Publication No. 20220033758 (published on Feb. 3, 2022), the contents of which is incorporated herein by reference in its entirety.

The geometry of the in situ-generated barrier can be selected to facilitate the cell exportation. Without intending to be bound by any theory, in some embodiments, the in situ-generated barrier is designed to have a structurally vulnerable portion so that, upon application of a threshold pressure (for instance, a force generated by the bubble created by the laser illumination), the in situ-generated barrier can be deformed, flipped or slipped from its original position, or changed of position resulting in an open space for a cell to be moved through. In other words, in those embodiments, the in situ-generated barrier will no longer impede or block the cell after the laser illumination.

In some embodiments, the in situ-generated barrier comprises one or more discrete sections, each of which is moveably connected to one or more surfaces of the chamber. As used here, “moveably connected” describes that the discrete section can be moved upon a threshold pressure. Therefore, application of a threshold pressure to the one or more discrete sections of the in situ-generated barrier moves at least one of the one or more discrete sections with respect to the one or more surfaces of the chamber and thereby creates an opening in the enclosed culture area. The opening can facilitate the exportation of the cell out of the chamber. It may be desired to select a hydrogel barrier design that provides non-uniformity in a center point of the width of the chamber, decreasing the width, thickness or height of the hydrogel gels/segments comprising the barrier as the forces created by the laser illumination are directed more forcefully there.

In some embodiments, the in situ-generated barrier comprises two or more discrete sections, wherein adjacent sections are separated from one another by a gap. In some embodiments, the in situ-generated barrier consists of (or consists essentially of) two discrete sections which are separated from one another by a gap. In some embodiments, the gap can be designed to cross the in situ-generated barrier and have an axis aligning with an axis of the chamber at an angle of 0°, 5°, 10°, 15°, 20°, 30°, 40°, 50°, 60°, 70°, 80°, or 90°, or any number between any two of listed numbers. In some embodiments, the in situ-generated barrier can have one, two, or more gaps, each of which may be on the order of the diameter of a single cell size, for example, 0.1× to 3.0×, 0.2× to 2.5×, 0.3× to 2.0×, 0.4× to 1.5×, 0.5× to 1.0×, or any range defined by two of the foregoing endpoints, where x is the average diameter of the cell.

In some embodiments, the structurally vulnerable portion can be of a suitable thickness. For instance, in some embodiments, a portion of the in situ-generated barrier has a thickness that is smaller than the height of the chamber.

In some embodiments, the in situ-generated barrier comprises a non-uniform thickness with respect to an axis of the chamber such that a portion of the in situ-generated barrier is less thick than other portions of the in situ-generated barrier. In some embodiments, the less thick portion of the in situ-generated barrier has a thickness that is smaller than the height of the chamber.

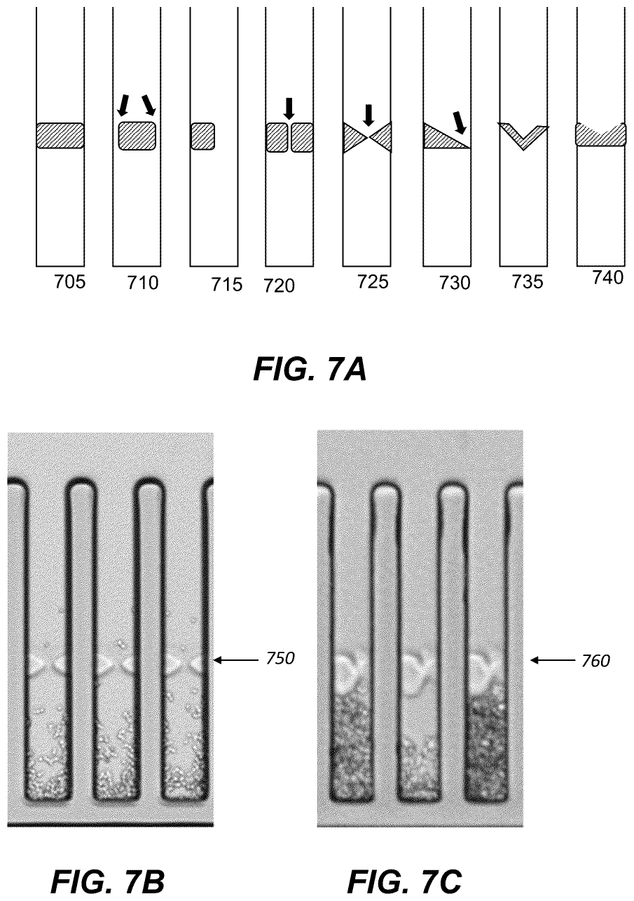

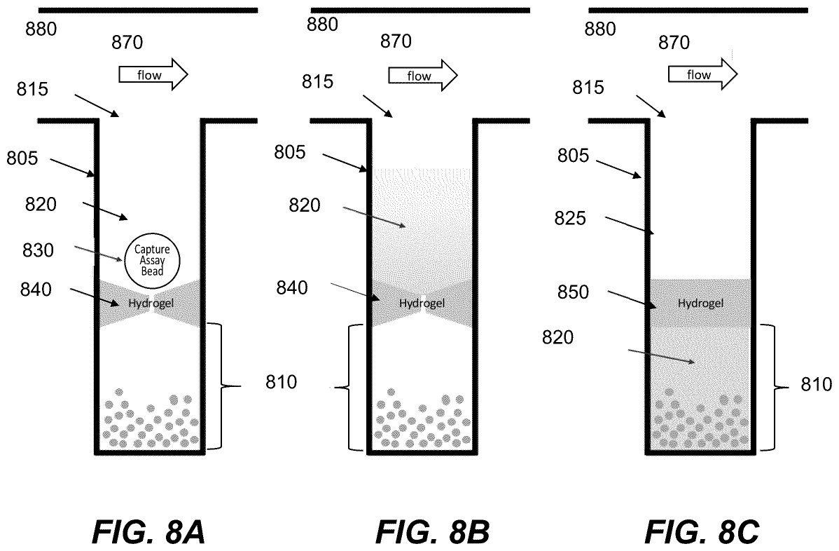

Accordingly, various exemplary shapes of in situ-generated barrier can be formed within the chamber, as shown in FIG. 7A. These include, but are not limited to a rectangular fully capping barrier (i.e. a fully sealed cap, 705, a center bar 710, a rectangular half bar 715, a side bars (two discrete side rectangular bars separated by a gap and extended from both sides of the wall), a “bowtie” 725 (two discrete side triangular bars separated by a gap and extended from both sides of the wall). Alternatively, the bowtie barrier can be formed by two discrete side trapezoid bars as shown in FIG. 8A or FIG. 8B), a single triangular bar 730, a V-shaped bar 735, and a V-cap (a rectangular bar having a v shaped depression on the side of the barrier facing the opening to the channel) 740. The center bar has two gaps (arrowed) respectively on each side between the walls of the chamber. The side bars, bowtie and single triangle bar both have a single gap (arrowed). The size of the gap can be 5, 6, 7, 8, 9, 10, 11, 12, 13, 14, 15, 16, 17, 18, 19, 20, 21, 22, 23, 24, 25, 26, 27, 28, 29, 30 microns or any range defined by two of the foregoing endpoints.

Further the size of the components of the hydrogel barrier may vary considerably. As shown in the photographs of FIGS. 7B-7C), where two embodiments of the “bowtie” barrier are shown. These examples of nonuniform barriers may have a variety of dimensions that are nonuniform. Non-uniformity may be located in the width (pen wall to pen wall dimension), in the “thickness” dimension (from distal-to-pen opening to proximal-to-pen opening dimension), and/or the “height” dimension (from inner surface of the substrate to the inner surface of the cover of the microfluidic device, e.g., z-dimension). The non-uniformity in the “thickness” dimension, e.g., the center of the “bowtie”, provides a convenient and standard location for bead disposition for the assay. The “bowtie” barrier may have a “thickness” at its largest dimension of the triangular shaped individual gel form of about 15 microns. This dimension is not limiting, and the “thickness” of the bowtie may be less than about 15 microns, less than about 12 microns, less than about 10 microns, less than about 8 microns, or less than about 5 microns. In other embodiments, the “thickness” of a triangular segment of the “bowtie” barrier may be more than about 10 microns, more than about 15 microns, or more than about 18 microns. As shown in FIG. 7B-7C, two different embodiments of hydrogel barriers having a “bowtie” non-uniform shape are shown, where the dimension 760 of the triangular segment forming the barrier (“thickness” dimension) is larger in the barrier of FIG. 7C than the dimension of the segment 750 of the barrier of FIG. 7B. Since the pens are of the same width, it can be seen that the barrier formed in FIG. 7B has a distinct gap between the two barriers, while the barrier of FIG. 7C does not have an obvious gap. While in the embodiment of FIG. 7B, a few cells have escaped the culture area below the barrier, the barrier of FIG. 7C does not permit cells to move past the barrier.

However, the use of hydrogel barriers does not limit the ability to selectively export cells from chambers having hydrogel barriers. As discussed in detail in Example 2-5, and shown in FIGS. 25A-25F, export of desired cells, at a user-defined timepoint may be performed.

Reporter molecule. Methods of assaying an intrinsic diffusion gradient and/or quantifying a level of secretion of a biological molecule by a biological micro-object(s) disclosed herein can comprise the use of one or more reporter molecules (e.g., detection reagents). In certain embodiments, such reporter molecules can be configured to: covalently or non-covalently bind to a secreted analyte of interest; and generate a signal that can be detected (e.g., using imaging). In some embodiments, the signal is proportional to one or more of the amounts of accumulated reporter molecule/RMSA complex resulting from one or more of: the secretion rate of a biological micro-object, the number of biological micro-objects, and/or the fraction bound of the analyte.

A reporter molecule typically includes a binding component configured to bind the secreted analyte of interest. Thus, the binding component may be any suitable binding partner capable of specifically binding to the secreted analyte of interest (e.g., with a binding constant less than 10 micromolar). As used herein, specific binding refers to a preference for binding the secreted analyte of interest over one or more other components of the system (e.g., one or more components on or within the microfluidic device). The binding component may comprise a protein, a peptide, a nucleic acid, a small organic molecule, or any combination thereof.

In some embodiments, the reporter molecule may be multi-valent, comprising more than one binding component such that the reporter molecule is able to bind more than one copy of the secreted analyte of interest or to bind more than one member of a group of secreted analytes. The stoichiometry of the reporter molecule-secreted analyte (RMSA) complex can therefore vary. One or more reporter molecules may bind to one or more secreted analytes, and additionally or alternatively one or more secreted analytes may bind to one or more reporter molecules. Thus, for example, a reporter molecule that binds a single copy of the secreted analyte may form an RMSA complex with a 1:1 stoichiometry. Alternatively, the RMSA complex may have a stoichiometric ratio of 2:1, 3:1, 4:1, 1:2, 1:3, 1:4, 2:2, 4:2, 2:4, etc. of reporter molecule: secreted analyte.

The reporter molecule may have any suitable molecular weight, provided that the reporter molecule is soluble and capable of diffusing in media disposed within the microfluidic device. For example, the reporter molecule may have a molecular weight that is about 10%, 20%, 30%, 40%, 50%, 60%, 70%, 80%, 90%, or about the same as the molecular weight of the secreted analyte of interest. Alternatively, the reporter molecule may have a molecular weight that is greater than the molecule weight on the secreted analyte of interest.

In some embodiments, the analyte of interest may be an antibody (or a fragment thereof) and the reporter molecule may comprise a binding component suitable for binding to antibodies (or fragments thereof). In some embodiments, the binding component of the reporter molecule may bind to an antibody Fc region (e.g., the Fc region of an IgG antibody). Thus, for example, the binding component of the reporter molecule may include a peptide, protein, aptamer, etc. configured to bind regions of an antibody (e.g., an IgG antibody). Molecules capable of specifically binding antibodies are well-known in the art. See, e.g., International Publication Nos. WO 2017/181135, filed on Apr. 14, 2017, and WO 2021/183458, Mar. 8, 2021, the contents of which is incorporated herein by reference in its entirety.

Detectable label. In some embodiments, the binding component of the reporter molecule may intrinsically possess the ability to generate a detectable signal, such as a visible, luminescent, phosphorescent, or fluorescent signal. In other embodiments, the reporter molecule may comprise a detectable label, which may be covalently attached directly or indirectly to the binding component of the reporter molecule. In other embodiments, the reporter molecule may comprise a detectable label that binds non-covalently to the binding component of the reporter molecule. The detectable label may be a visible, luminescent, phosphorescent, or fluorescent detectable label. In some embodiments, the detectable label may be a fluorescent label. Any suitable fluorescent label may be used, including but not limited to fluorescein, rhodamine, cyanine, phenanthrene or any other class of fluorescent dye.

In some embodiments, the binding component of the reporter molecule may comprise a capture oligonucleotide and the detectable label may be an intercalating dye. For example, the reporter molecule may comprise a capture oligonucleotide and either an intrinsic or extrinsic fluorescent dye may be the detectable label. In some embodiments, the detectable label of the reporter molecule may not be detectable until the capture oligonucleotide binds the analyte of interest, as might be the case when the detectable label is an intercalating dye. More generally, in some embodiments, a detectable label of a reporter molecule may not be detectable until after the RMSA complex has formed, as formation of the RMSA complex may shift the detectable signal to a new wavelength does not present prior to binding.

As used herein, “a signal associated with the detectable label” or similar phases refers to a signal that is directly or indirectly emitted by the detectable label within an area of interest. In some embodiments, the signal associated with the detectable label is detected after a steady state equilibrium is reached. In other embodiments, the signal associated with the detectable label is detected while perfusing another fluidic medium that does not comprise the reporter molecule into the flow region.

Cell culture and induction. The cell to be evaluated by any of the methods of the present disclosure is not limited. In some embodiments, the cell can be a eukaryotic cell or a prokaryotic cell. In certain embodiments, the cell is an animal cell, a plant cell, fungal cell, or a bacteria cell. In some embodiments, the cell is a fungal cell. In some embodiments, the cell is a yeast cell, including but not limited to a Saccharomyces cell (e.g., Saccharomyces cerevisiae) or a Pichia cell (e.g., Pichia pastoris). In some other embodiments, the cell is a bacterial cell, which may be, but is not limited to Escherichia coli (E. coli), or any other bacterial cell that may be engineered to produce a desired bioproduct.

In some embodiments, the cell is maintained in the chamber. In some embodiments, the cell is cultured and expands (i.e., proliferates) in the chamber into a clonal population. In certain embodiments, the cell expands to a number of cells of which the secretion of a molecule of interest is of a level sufficient to be detected by the method of the present disclosure.

In some embodiments, culturing in the chamber of the microfluidic device may include culturing a cell or a clonal population thereof within a volume of medium less than 5 nanoliters. In some variations, the macroscale reactor of which the bioproductivity is predictive of may have a volume of 100 mL, 1 L, 10 L, 100 L or more. In some variations, culturing in the chamber of the microfluidic device may include culturing under substantially similar conditions to conditions of culturing in the macroscale reactor.

In some embodiments, the cell is cultured in the presence of a selected level of a component of a fluidic medium. In some embodiments, the component may be a nutrient for the clonal population of the cells. In some embodiments, the selected level of the component may be a growth limiting level of the component.

In some embodiments, cell is induced to secrete the molecule of interest. The induction can be performed according to the general knowledge of the cell. In some embodiments that the cell is engineered to secrete the molecule of interest, the induction can be performed based on the nature of the promoter constructed for the expression of the molecule of interest in the cell. In some embodiments that the cell is a yeast cell engineered with an AOXI promoter, a BMMY medium or a BMIM medium is introduced into the flow region and allows to diffuse into the chamber to induce the secretion. In some embodiments, the fluidic medium used for induction comprises at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, or about 15% methanol v/v. Preferably, a BMIM medium is used to induce the secretion. In certain embodiments, the medium used for induction is oxygenated with at least about 10%, 15%, 20%, 25%, 30%, 35%, 40%, 45%, 50%, 55%, or 60% of oxygen.

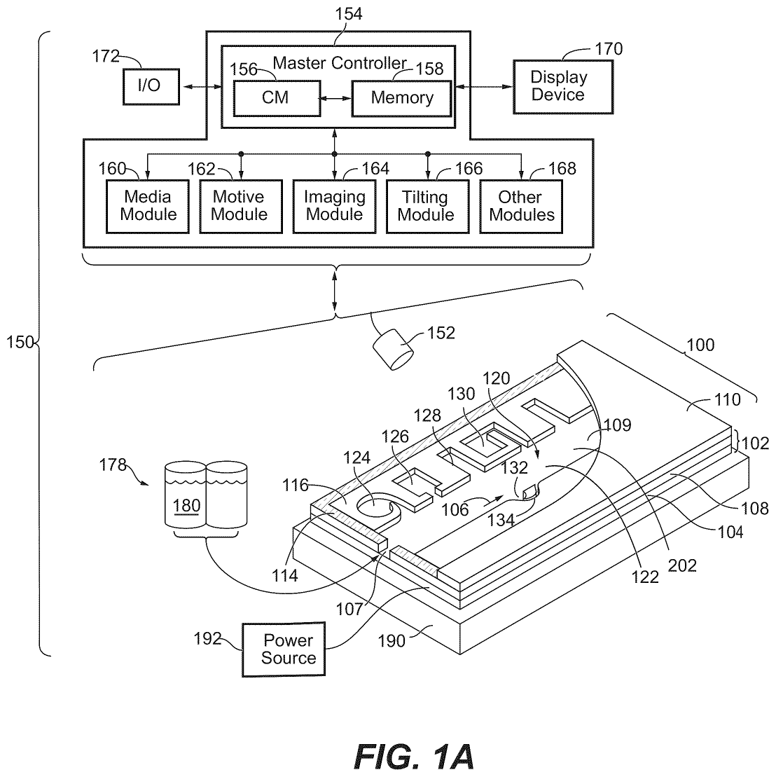

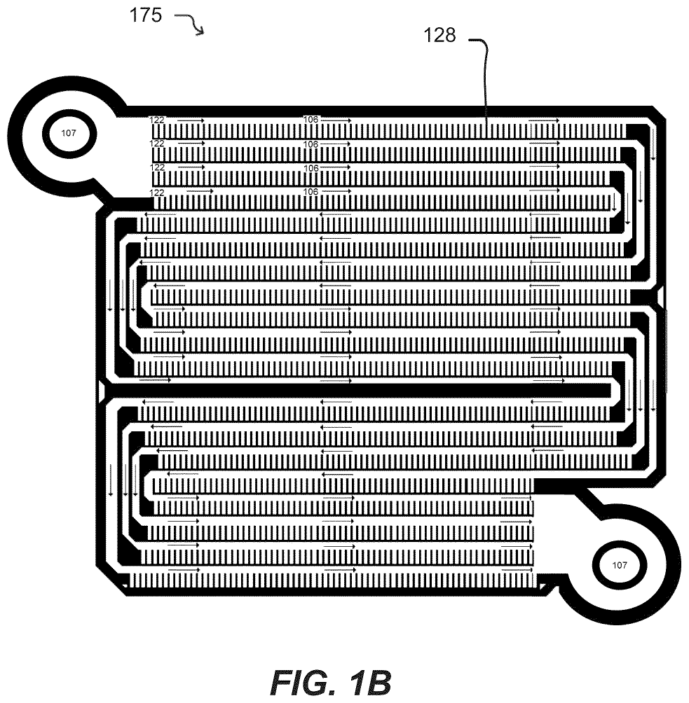

Assays. In light of the foregoing, the methods of the present disclosure provide a variety of different approaches to evaluate bioproductivity of a cell. Three of those variations are shown in FIGS. 8A-8C. FIG. 8A shows a bead assay. FIG. 8B shows a diffusion gradient assay, and FIG. 8C shows an accumulation assay herein. FIGS. 8A-8C are top views of the chambers 805, and the chambers normally are disposed horizontally, similarly to the sequestration pens 124, 126, 128, 130 shown in FIG. 1A. The chambers 805 open laterally from the flow region, e.g., channel 880, where flow 870 is contained. As shown in FIGS. 8A-8C, each chamber 805 has an in situ generated hydrogel barrier 840 or 850 formed within the chamber, dividing the chamber into two areas. The hydrogel barrier is generally introduced after cell(s) have already been introduced into the chamber. The first area is a culturing area 810, which is disposed distal from the hydrogel barrier, in relationship to the opening 815 of the chamber to the channel 880. This culturing area 810 is an unswept region of chamber, and no flow 870 from the channel 880 flows directly into that part of the chamber, even if the hydrogel barrier were not there. The second area 820 created by installation of the hydrogel barrier, is an assaying area and is located to the proximal side (in relation to the opening 815 of the chamber to the channel) of the hydrogel barrier 840 or 850.

As shown in FIG. 8A, an assay is provided (“bead assay”), where detection within the assay area 820 is performed by capturing secreted analyte to a bead 830. The secreted analyte diffuses either through the hydrogel barrier 840 or through a small gap between segments of the hydrogel barrier that is shown in FIG. 8A.

As shown in FIG. 8B, an assay is provided (“diffusion gradient assay”) where detection of the secreted analyte occurs in the same region as that of the bead assay, but no bead is provided. Alternatively, an area of interest is selected within the assay area 820, and a labelling reagent is permitted to diffuse into the assay area and bind to the analyte of interest. A gradient profile of the labelled analyte, as it diffuses towards the opening of the chamber and into the channel can be obtained, providing an amount of fluorescence detected that is proportional to the amount of analyte. The detected amount of fluorescence may be used as a raw total, raw ranking or a raw score. The detected signal may also be further processed to provide a normalized (for biomass), corrected (for background unbound labelling reagent and the like), and/or normalized against other detected signals in other chambers on the chip to produce a total or score. Further details of how to conduct a diffusion assay are described below at the section entitled General Diffusion Assay Techniques and in the references described therein.

In FIG. 8C, a third type of assay is shown. In this assay (“accumulation assay”), a hydrogel barrier 850 that spans the width of the chamber without interruption is introduced into the chamber. The chamber is divided into two areas, a culturing area 810 and second area 825 that is separated from cells. The secreted analyte is permitted to accumulate within the culturing area 810 and an assay area 820 is selected within the culturing area. This permits a cell/colony of cells to secrete an analyte and accumulate within area 820. The hydrogel barrier in this case does not readily permit diffusion of the secreted analyte across the hydrogel, so the secreted analyte will accumulate. This is particularly useful if the rate of secretion of the analyte is low. The labelling reagent is introduced from the channel into the chamber by diffusion. Because the labelling reagent is selected to have a smaller molecular weight (size), the labelling reagent can diffuse from area 825 through the hydrogel barrier 850, and into the culturing area 810/Assay area 820 and bind to the secreted analyte. The labelled analyte may be detected and produce a raw total, ranking or score. The detected signal may also be further processed to provide a normalized (for biomass), corrected (for background unbound labelling reagent and the like), and/or normalized against other detected signals in other chambers on the chip.

Without intending to be bound by theory, the bead assay may be used for evaluating a predicted secretion level of about 0.01 mg/liter to about 0.25 mg/liter; a diffusion gradient assay may be used for a predicted secretion level of from about 20 micrograms/ml (about 1 picogram/cell/day or about 7 attomoles/cell/day) to about 2500 micrograms/ml (about 17 picograms/cell/day); and an accumulation assay may be used for a predicted secretion level of about 0.1 mg/liter to more than about 2.5 mg/liter, or more than 25 mg/liter or more.

Many different variations of assays may be used to assess bioproductivity of a cell or a clonal population derived therefrom, and alternatives are described herein and may be adapted as suitable for the particular use case. Additionally, further variants of the diffusion assay (open pen assay) and an immiscible fluid sealed pen assay are described below.

Accumulation assay. In light of the foregoing, an accumulation assay is provided. In some embodiments, the in situ-generated barrier is formed at middle position of the chamber (FIG. 8C) and defines an enclosed culture area at the distal end of the chamber. The in situ-generated barrier has a first permeability with respect to the analyte and a second permeability with respect to the reporter molecule. In some embodiments, the first permeability is lower than the second permeability. In other words, a diffusion rate of the analyte diffusing out of the barrier is slower than a diffusion rate of the reporter molecule diffusing. Given that the RMSA complex is a larger than the secreted analyte, its diffusion rate would be even slower, resulting in the RMSA complex accumulating with the enclosed culture area. In some embodiments, to offer a better accumulation of the RMSA complex, the in situ-generated barrier has a porosity that impedes diffusion of the RMSA complex through the in situ-generated barrier. In certain embodiments, the porosity of the in situ-generated barrier substantially prevents diffusion of the RMSA complex through the in situ-generated barrier. In some embodiments, the area of interest is within the enclosed culture area. In some embodiments, the area of interest is within a cell-free area of the enclosed culture area. In some other embodiments, the area of interest is within an area of the enclosed culture area containing the cells. In some embodiments, the area of interest does not include a portion of the in situ-generated barrier.

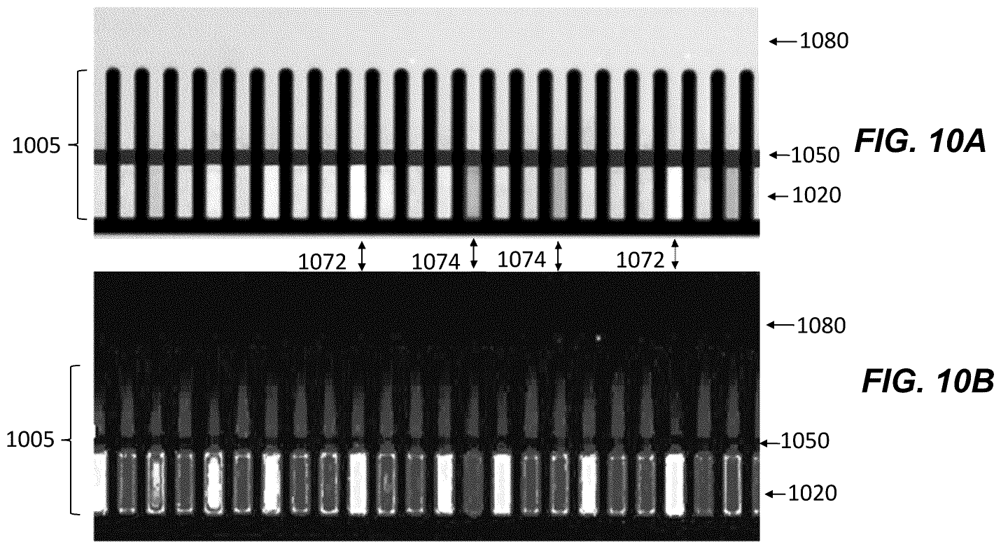

An image can be taken after a steady state equilibrium is reached as described above for detecting a signal associated with the detectable label of the reporter molecule. As shown in FIGS. 10A and 10B, fluorescence images of the chambers 1005, which open to channel 1080, where hydrogel barriers 1050 have been introduced in each chamber at the same point distal from the opening of the chamber to the channel. FIG. 10A is taken at a timepoint when labelled reporter molecule is being flowed through channel 1080 and has diffused into the pens 1005 Fluorescent signal is observed in both the channel and chambers, and additionally in the culturing area 1020. Here, the free reporter molecule (i.e., unbound) in the channel and the chamber and the RMSA both emit fluorescent signals. A range of intensities of signal can be observed at the distal end of the chambers, representing different productivities of the cells cultured in each chamber. Among them, better producers can be identified, the chambers 1072 comprising two of which are annotated for both FIGS. 10A and 10B, and the cells can be exported.

A fluorescence image can also be taken at a later timepoint, while flushing a fluidic medium that does not comprise the reporter molecule into the microfluidic device as described above, as shown in FIG. 10B. The channel in this image is dark because the fluidic medium that is perfused at this moment does not comprise the reporter molecule. The in situ-generated barrier 1050 is not clearly seen in this image but it is at the same position as indicated in FIG. 10A. A range of intensities of signal can also be observed at the distal end of the chambers in the culturing area 1020, showing differential signal still accumulated within the culturing areas below the barrier 1050. These show different productivities of the cells cultured in each chamber. It can be seen that the signal in the chambers 1072 comprising the positive secretor is brighter than other chambers, correlating with the observation from FIG. 10A. Alternatively, two poorly performing colonies are identified at chambers 1074, where the image is slightly less fluorescent in FIG. 10A and very dim in FIG. 10B. This demonstrates that while substantial amounts of reporter molecule have diffused in the respective culturing areas 1020, once flushing with medium carrying no labelling molecule has been performed, unbound reporter molecule has diffused out and little to no bound RMSA is found.

Diffusion gradient assay. In light of the foregoing, a diffusion gradient assay is provided. In some embodiments, the in situ-generated barrier is formed at middle position of the chamber (FIG. 8B) and defines an enclosed culture area at the distal end of the chamber and an assay area at the proximal end of the chamber. In some embodiments, the area of interest is located within the chamber but not within the enclosed culture area, for instance, within the assay area. In certain embodiments, the area of interest is within a cell-free region of the chamber. In some embodiments, the area of interest does not include a portion of the in situ-generated barrier.

The in situ-generated barrier has a first permeability with respect to the analyte and a second permeability with respect to the reporter molecule. In some embodiments, the first permeability is lower than the second permeability. Preferably, the in situ-generated barrier has a porosity that allows diffusion of the RMSA complex through the in situ-generated barrier. In some embodiments, the in situ-generated barrier used in the diffusion gradient assay comprises a gap (e.g., a bowtie barrier as described above) through which the RMSA complex can diffuse (e.g., and thereby cross the in situ-generated barrier). Because the analyte is secreted by the cell in the chamber and diffuse from the distal end of the chamber toward the proximal end thereof, a gradient of signal associated with the detectable label can be observed within the assay area.

Further details of diffusion gradient assays and data analysis thereof is provided below at the section entitled General Diffusion Assay Techniques.

Bead assay. In light of the foregoing, a bead assay is provided, as shown in FIG. 8A. The methods for the bead assay includes: disposing the cell into a chamber of a microfluidic device, the microfluidic device having a microfluidic circuit comprising a flow region and the chamber, wherein the chamber comprising an opening to the flow region; forming an in situ-generated barrier within the chamber, wherein the in situ-generated barrier defines within the chamber an assay area and an enclosed culture area for culturing the cell; disposing a micro-object in the assay area of the chamber, wherein the micro-object comprises a capture moiety configured to bind an analyte secreted by the cell; allowing the cell to secreting the analyte; introducing a first fluidic medium comprising a reporter molecule into the flow region, wherein the reporter molecule comprises a first detectable label and a binding component configured to bind to the analyte; and detecting a first signal associated with the first detectable label within an area of interest within the microfluidic circuit, thereby evaluating the bioproductivity of the cell.

In some embodiments, the in situ-generated barrier has a porosity that allows diffusion of the analyte through the in situ-generated barrier so that the analyte can bind the micro-object within the assay area. In some embodiments, the in situ-generated barrier used in the diffusion gradient assay comprises a gap (e.g., a bowtie barrier as described above) through which the analyte can diffuse (e.g., and thereby cross the in situ-generated barrier) while the micro-object cannot pass through the gap.

In some embodiments, the area of interest is located within the chamber but not within the enclosed culture area, for instance, within the assay area. In certain embodiments, the area of interest is within a cell-free region of the chamber. In some embodiments, the area of interest does not include a portion of the in situ-generated barrier.

In some embodiments, the micro-object is a surface coated with the capture moiety. As used herein, “capture moiety” is a chemical or biological species, functionality, or motif that provides a recognition site for the analyte. In certain embodiments, the micro-object is a bead coated with the capture moiety. The bead may be made of any suitable material, such as polymer, metal, ceramic, glass, or any combination thereof. The bead may be magnetic or may not be magnetic.

In some embodiments, the analyte comprises a first tag and a second tag. The first tag is configured to be bound by the capture moiety of the micro-object. The second tag is configured to be bound by the binding component of the reporter molecule. Without intending to be bound by theories, the binding between the micro-object and the analyte accumulates the analyte on the surface of the micro-object and therefore amplifies the signal associated with the detectable label of the reporter molecule.

In some embodiments, the capture moiety comprises a peptide or a protein. In some embodiments, the first tag can be an epitope tag (e.g., a protein tag) including, but not limited to FLAG-tag, E-tag, Myc-tag, T7, NE-tag, Spot-tag, V5-tag, VSV-tag, and the like, which are known in the art. In some embodiments, the first tag is a FLAG tag (for example, Thermo Fisher Cat. No. 701629), and the capture moiety is an anti-FLAG antibody. In some variations, the micro-object includes a metal chelate species which can recognize a protein tag such as a His-tag (poly Histidine, which is chelated by a nickel or cobalt chelate. The nickel chelate may be Ni(II)-nitrilotriacetic acid (Ni-NTA). Another metal chelating tag is TC tag (which is bound by FLASH or ReAsH biarsenical compounds). Any suitable protein tag may be used to capture secreted analyte to the capture moiety of the micro-object.

In some embodiments, the bead is a bead coated with streptavidin covalently or noncovalently, and the capture moiety comprises a biotin functionality, which can bind the streptavidin of the bead. In some embodiments, except for streptavidin/biotin, other coupling groups can be used, including but not limited to, biotin/avidin, biotin/NeutrAvidin, and digoxygenin/anti-digoxygenin.

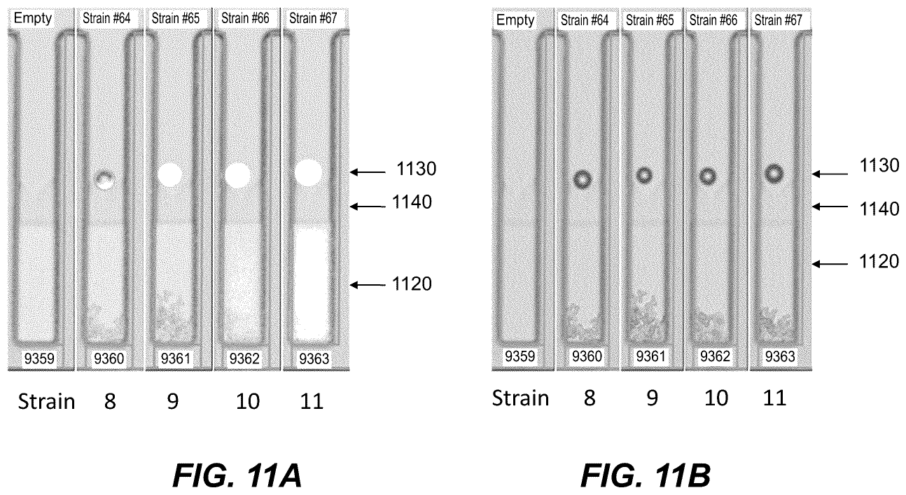

FIG. 11A and FIG. 11B shows images of an exemplary bead assay of the present disclosure. FIG. 11A is a fluorescent image, and FIG. 11B is a brightfield image. In this experiment, four strains (Strain 8, Strain 9, Strain 10, Strain 11) were used and their bioproductivities had been pre-determined. Their bioproductivities were consistent with the numbering of the strains with Strain 11 being the best producer among the four and Strain 8 being the least productive among them. An in situ-generated barrier 1140 having a v-shaped cap shape, like 740 of FIG. 7A was formed at mid-pen to contain the cells within the enclosed culture area 1120. The bead 1130 was positioned in the assay area and disposed between the v-shape of the barrier. As shown in the fluorescent FIG. 11A, the bead of Strain 11 exhibited the stronger intensity compared to the others, e.g., had the most reporter molecule labeling captured analyte. The bead of Strain 8 had the lowest intensity, which is consistent with the pre-determined bioproductivities. Additionally, it is noticeable that the bead of Strain 8 was brighter at the side close to the enclosed culture area in the fluorescence image FIG. 11A, indicating the RMSA was diffusing in the direction from the hydrogel barrier and did not saturate the side of the bead 1130 that faces the opening of the chamber to the channel, only slightly shown in these figures.

Recovery step in bead assay. In some embodiments when a bead assay is performed, a bead is introduced and penned into the chamber before the induction. In some embodiments, a fluidic medium comprising the bead is introduced into the microfluidic channel and the perfusion of the fluidic medium is stopped for applying OEP to pen the bead into the chamber. During the period of penning the bead, because the fluidic medium is stagnated, e.g., stopped, there is no replenishment of nutrient for the cell and the waste produced by the cell diffuses into the channel, but is not swept away. Therefore, in some embodiments, after the bead is penned, another culture period (i.e., a recovery step) is performed to allow the cell to recover to normal status. In some embodiments, the recovery step comprises introducing a culture medium into the flow region of the microfluidic device and continue the perfusion of the culture medium for at least about 10 min, 20 min, 30 min, 40 min, 50 min, 1 hr, 1.5 hr, 2 hr, 2.5 hr, 3 hr, 3.5 hr, 4 hr, 4.5 hr, 5 hr, 5.5 hr, or 6 hr, or any range defined by two of the foregoing endpoints.

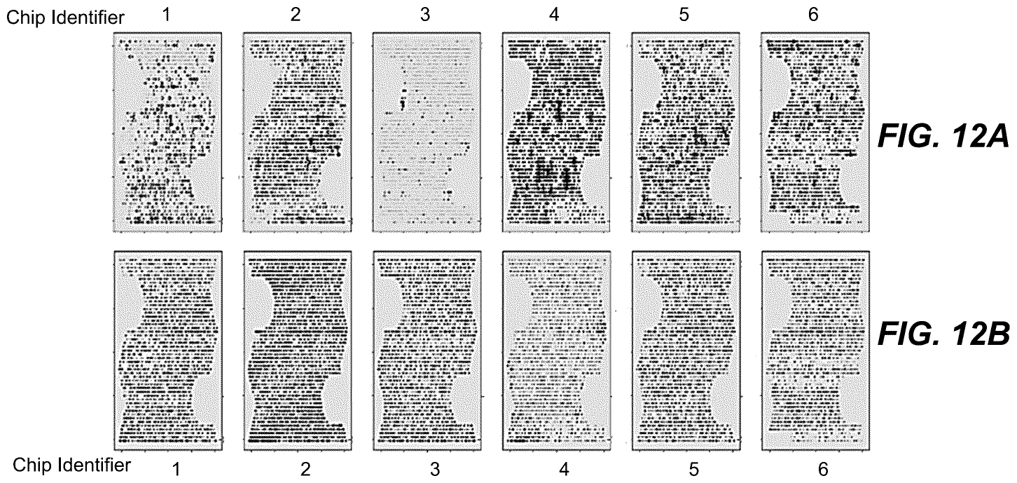

FIG. 12A and FIG. 12B show an experiment comparing the induction efficiency with and without the recovery step. In this experiment, a pre-determined high producer yeast strain was introduced and cultured on chips 1 through 6. After beads were penned into respective chamber, a recovery step of perfusing BMGY medium with 40%02 and 80% air/media fraction (cycle duration was 10 minutes) was performed on chips #4, 5, and 6 for 3 hours. Chips #1, 2, and 3 were control chips where no recovery process was performed. Then, a bead assay was performed on each chip and the fluorescent signals were detected. The fluorescent signal intensity is shown for each chip for each chamber across the entire chip.

FIG. 12A shows the bead score maps of each chip. Stronger signals (darker signal) in chambers indicate higher induction. FIG. 12B shows OD maps of each chip. Stronger signals (darker signal) in chamber indicates higher biomass, e.g., more cells in the chambers. The bead score maps of the three chips not utilizing the recovery process (Chips 1, 2, and 3) show non-uniform and poor induction even if the three chips all had cells in chambers, as shown in FIG. 12B. In comparison, the three chips (Chips 4, 5, and 6) utilizing the recovery process show uniformed and better induction. The result suggests the recovery step is beneficial to mitigate the influence of bead penning step and improve the induction efficiency.

Signal normalization and correction. The detected signal associated with the detectable label of the report molecule corresponds to the bioproductivity of the cell to be evaluated. Generally, signals obtained from cells cultured and evaluated under substantially same conditions can be compared with each other to determine which one of them offers better bioproductivity. In some embodiments, considering that a cell that is expanding faster does not necessarily secrete more, the methods of the present disclosure further comprise normalizing the detected signal associated with the detectable label with the number of the cells existing in the chamber to obtain a relative specific productivity. In some embodiments, the number of the cells is represented by the biomass of the cells. In some embodiments, the biomass is measured before an assay is performed; for example, right before introducing the reporter molecule into the microfluidic device. The biomass can be measured according to the methods described in the present disclosure.

In some embodiments, the signal is further corrected with a reference signal. In those embodiments, the methods of the present disclosure further comprise introducing a reference molecule into the flow region, wherein the reference molecule comprises a second detectable label different from the first detectable label; allowing the reference molecule to diffuse into the chamber; and detecting a reference signal associated with the second detectable label. The reference molecule does not bind the analyte and is used to provide a basic level of signal under the optical and cultural environment within the microfluidic device.

In many embodiments, the reference molecule is preferable to behave similarly as the reporter molecule behaves except that the reference molecule does not bind the secreted analyte. To be more specific, for example, if the reporter molecule enters the cell to be evaluated to bind the analyte, the reference molecule should enter the cell too except that the reference molecule does not bind the secreted analyte. In other examples, if the reporter molecule binds a surface of the cell, the reference molecule should bind to the surface too. In more other examples, if the reporter molecule does not bind or enter the cell, the reference molecule should not interact with the cell either. Moreover, the diffusion rate of the reference molecule in the environment of the chamber is preferable to be similar or substantially the same with that of the reporter molecule. In one of the examples described herein, the reporter molecule is a nanobody of 30 kDa having a globular structure, and the reference molecule is a 10 kDa dextran exhibiting similar diffusion behavior because of its linear structure.

In many embodiments, the second detectable label comprises a visible, luminescent, phosphorescent, or fluorescent detectable label. In some embodiments, the reference molecule is introduced together with the reporter molecule in a fluidic medium. In some embodiments, an image is taken for detecting the signal of the reference molecule after a steady state equilibrium is reached.

Selection. The methods of the present disclosure provide information for selecting or deselecting a cell cultured in the microfluidic device based on its bioproductivity. In some embodiments, the microfluidic device comprises a first chamber and a second chamber. In those embodiments, a first cell and a second cell are disposed into the first chamber and the second chamber respectively, and they are allowed to secret a molecule of interest. A first signal and a second signal are detected respectively. In some embodiments, the first signal and the second signal are compared with each other, and one of the first cell and the second cell is selected for having a stronger signal, which corresponds to a higher bioproductivity. In some embodiments, both of the first signal and the second signal are compared with a threshold, and one or both of the first cell and the second cell are selected if their signals are stronger than the threshold; or none of them is selected if their signals are weaker than the threshold.

The threshold can be determined from a reference cell of which the bioproductivity was pre-determined. The reference cell might be cultured in another chamber, i.e., a third chamber, of the same microfluidic device, or was cultured in a different microfluidic device under same or substantially the same conditions and has its signal normalized and corrected for comparison.

Kit. In light of the foregoing, a kit for evaluating bioproductivity of a cell is provided to perform the methods of the present disclosure. The kit comprises a reporter molecule comprising a first detectable label and a binding component configured to bind an analyte secreted by a cell to form a reporter molecule: secreted analyte complex (RMSA complex); and a prepolymer configured to be controllably activated to form an in situ-generated barrier comprising a solidified polymer network, wherein the in situ-generated barrier has a porosity that substantially prevents the cell from crossing through the in situ-generated barrier.

In some embodiments, the kit further comprises a reference molecule comprising a second detectable label different from the first detectable label, and further wherein the reference molecule does not bind the analyte. The reference molecule is as described above.

In some embodiments, the kit further comprises a microfluidic device comprising a microfluidic circuit comprising a flow region and a chamber, wherein the chamber comprises an opening to the flow region. The microfluidic device can be as described herein.

Microfluidic Prediction of Yeast Productivity. In light of the foregoing, methods are therefore provided for improving yeast cell bioproductivity including; culturing one or more yeast cells within each of a plurality of chambers of a microfluidic device, where the microfluidic device includes a flow region configured to flow a first fluidic medium and the chamber opens to the flow region; expanding the one or more yeast cells to form a population of yeast cells in each of the plurality of chambers; monitoring the production of a biomolecule by the population of yeast cells in each of the plurality of chambers; and predicting one or more populations of yeast cells configured to effectively produce the biomolecules. These methods may be used to screen mutant strains of a yeast, which may be either randomly mutagenized or may be mutagenized according to pre-selected insertions into the yeast genome. Effectively producing the biomolecules may include one or more of: producing the biomolecules more rapidly than a parent, unmutagenized strain, producing the biomolecules utilizing less or less expensive feedstock than a parent unmutagenized strain, and producing the biomolecules more rapidly even in the presence of waste products than a parent unmutagenized strain.

In some embodiments, effectively producing the biomolecules may include effectively producing the biomolecules when culturing the population of yeast cells in a macroscale reactor.

In some variations, predicting may further include selecting one or more populations of yeast cells producing higher levels of the biomolecule relative to levels of production of the biomolecule of the population of yeast cells in the other chambers of the plurality of chambers.

In some embodiments, monitoring the production of the biomolecule may include detecting a detectable signal associated with the biomolecule. The detectable signal can be associated with a detectable label of a reporter molecule as described herein. In some variations, the biomolecule may be inherently detectable. In other variations, the biomolecule may be detectable upon labelling with a detectable label as described herein.

In some variations, the method further includes monitoring the production of the biomolecule at a time wherein the culturing conditions approximate pseudo steady state conditions.

In some embodiments, effectively producing the biomolecules may include effectively producing the biomolecules based on feedstock. In some other embodiments, effectively producing the biomolecules based on feedstock may include at least one of efficiently converting the feedstock to biomolecules and efficiently producing the biomolecules in the presence of byproduct accumulation.

The simultaneous culture of ˜10⁴⁻⁵ segregated colonies of differing genotypes, assay and selection of desired clonal populations may be achieved. The microfluidic system can employ light-actuated cell positioning (e.g., Opto-ElectroPositioning (OEP™), described herein, which can induce a dielectrophoretic force, which in this case is a negative dielectrophoretic force, to individually manipulate single cells by repelling the cells. In other embodiments, this force may be a positive dielectrophoretic force, which transports the cells by attracting them to the non-uniform dielectric field induced.

The phenotype of each colony can then be assessed by monitoring the growth and secretions using bright-field imaging and fluorescence-based assays. Once colonies bearing desirable phenotypes are identified, OEP can be used to assist in unpenning the cells, which can then be exported into microplates for further study.

Within the microfluidic device/system, the productivity of each strain could be directly monitored in real time during continuous culture, yielding phenotypes that correlated strongly (R²>0.8, p<0.0005) with behavior in industrially relevant bioreactor processes. This method allows a much closer approximation of a typical fed-batch fermentation than conventional batch-like droplet or microplate culture models, in addition to rich time-dependent data on growth and productivity.

Novel methods for identifying improved small molecule-producing strains are described herein, and rely on fluorescence-based detection of secreted product, from which per-cell productivities can be inferred. Although assays have previously been used to identify mammalian cell lines for antibody production, it was not known whether these methods could be applied to screening microbial strains for efficient small-molecule production. High-throughput (>10³ strains/week) strain screening based on small-molecule secretion rate under chemostat-like culture conditions has been demonstrated for the first time using the methods described herein. Using strains that produce a fluorescent product as a test case, it was found that assay scores arising from these assays correlate well with peak strain performance in 0.5- and 2.0-L pulse-fed bioreactors.

It was also discovered that the methods can be used to rapidly isolate strains from random whole-genome mutagenesis libraries that exhibit improved specific productivities in ambr250 bioreactors. In some embodiments, in an optimized two-tiered screening workflow described herein, reduced the timeline of library screening by 2-fold, obviated solid culture and colony-picking, and eliminated >95% of material waste produced in conventional microplate screening. Further the methods yielded mutants with substantially improved (by up to 85%) peak specific productivities in bioreactors. In certain embodiments, each screen of ˜5×10³ mutants was completed in under 8 days (including 5 days involving user intervention), saving ˜50-75% of the time required for conventional microplate-based screening methods.

Methods of Determining Bioreactor Performance. In metabolic engineering, specific productivity (qₚ; mol product·mol biomass⁻¹·hour⁻¹) and growth rate (u (Greek letter “Mu”); hour⁻¹) are two key variables intrinsic to the genotype of a strain under a set of fixed culture conditions. If a scaled-down screening system effectively models strain physiology in a lab-scale bioreactor, estimates of qₚ and μ can be used to predict aspects of bioreactor performance. Microfluidic culture conditions were empirically selected by optimizing the correlation between strain qₚ on the microfluidic system and in a conventional lab-scale fermentation platform. The assays used allow measurement of biomass and product secretion, which together enable inference of both qₚ and μ. In the particular embodiments described herein, conditions were not selected to optimize correlation of μ between the microfluidic device environment and the lab-scale bioreactor, but the methods are not so limited.

The values of qₚ and μ for a strain are industrially relevant because they are determinants of yield (Y; mol product·mol sugar fed⁻¹) and volumetric productivity (P; mol product·L⁻¹·hour⁻¹), which are two key factors in the cost of production during manufacturing. In this work, the methods described herein were evaluated by comparing assay values for strains to qₚ, Y, and P values observed from lab-scale bioreactor fermentations. These fermentations typically begin with a period of rapid growth, followed by a later stage of pseudo-steady-state production when nutrients are more restricted. In some embodiments of the methods described herein, the assays attempt to model the latter stage. Thus, the relevant performance metrics were taken from the interval 24-48 hours following bioreactor inoculation, after the end of the rapid growth phase.

Sealed Chamber Assay. In light of the foregoing, a method for assessing relative productivity of a detectable molecule by a population of yeast cells, in a microfluidic device having an enclosure comprising a channel and a plurality of chambers, each chamber of the plurality having an opening fluidically connecting the chamber to the channel, is provided. The method provides results correlating well with macroscale culture assays and permitting early, cost-effective screening. The method comprises disposing a yeast cell configured to produce the detectable molecule in each chamber of the plurality of chambers; flowing a first aqueous medium into the channel; culturing the yeast cell to expand to a clonal population of yeast cells; flowing a water immiscible fluidic medium into the channel, displacing substantially all of the first aqueous medium in the channel; monitoring over a period of time an increase of a signal from the detectable molecules produced by the clonal population of yeast cells in each chamber of the plurality; and determining a relative productivity for each clonal population of yeast cells.

In some embodiments, determining the relative productivity may include determining a relative productivity of the clonal population in the presence of increasing levels of byproducts from the clonal population. Although there are many ways to transduce a fluorescent signal in response to the local concentration of an analyte, detection is easiest when the product is intrinsically fluorescent. A set of S. cerevisiae strains engineered to produce a fluorescent small molecule was thus chosen to demonstrate the feasibility of screening for a secretion phenotype. Therefore, in some embodiments, the molecule may be inherently detectable. In other embodiments, the molecule is detectable upon labelling with a detectable label as described herein.

In some embodiments, the sealed chamber assay can be performed for about 10 min, about 20 min, about 30 min, about 40 min, about 50 min, about 60 min, about 2 h, about 4 h, about 6 h, about 8 h, about 10 h, about 12 h, about 14 h, about 16 h, about 18 h, about 20 h, about 22 h, about 24 h, about 26 h, about 28 h, about 30 h, about 32 h, about 34 h, or about 36 h. In some embodiments, the sealed chamber assay can be performed for 20 minutes (short term) to 24 hours (long term). In an embodiment of a 24-hour sealed chamber assay, four strains cultured gave endpoint fluorescence values that ranked them consistently with the relative titers attained in microplate models after carbon sources had been exhausted, demonstrating comparability with macroscale cultures.

In some embodiments, when the sealed chamber is performed for a long term (e.g., 24 hours), offers some interesting opportunities that warrant further exploration: (1) tracking growth and productivity under the constraint of limited feedstock provides a direct comparison of resource utilization from strain to strain, a readout complementary to the real-time productivity measurements from the open-chamber assay. (2) Because many commercial fermentations are run in fed-batch rather than continuous perfusion conditions, high product and byproduct accumulation can present an obstacle to high performance. Using a water immiscible fluidic medium to block the efflux of product may help to apply selective pressure in screening efforts to reduce feedback inhibition and increase product tolerance; likewise, blocking the efflux of toxic byproducts may help screen for strains that produce them at lower concentrations. Therefore, this sealed chamber format can be a complementary tool for assessing strain resilience to produce and by-product accumulation.

In some embodiments, the water immiscible fluidic medium comprises an alkane, a fluoroalkane, an oil, a hydrophobic polymer, or any combination thereof. Th oil can be a hydrophobic oil, including but not limited to, a silicone oil or a fluorinated oil. Specific examples of water immiscible fluidic media include isooctane (or heptamethyl nonane (HMN)), hexadecane, 2-(Trifluoromethyl)-3-ethoxydodecafluorohexane (HFE-7500, 3M™, Novec™), Fluorinert™ FC-40, (Aldrich Cat No. F9755), Fluorinert™ FC-70, (Aldrich Cat No. F9880), bis(2-ethylhexyl) carbonate (TEGOSOFT® DEC, (Evonik)), (Tridecafluoro-1,1,2,2,-tetrahydrooctyl) tetramethydisiloxane (Gelest, Cat #SIB1816.0), silicone oil (5 centistoke viscosity, Gelest Cat. #DMS-T05), and the like.

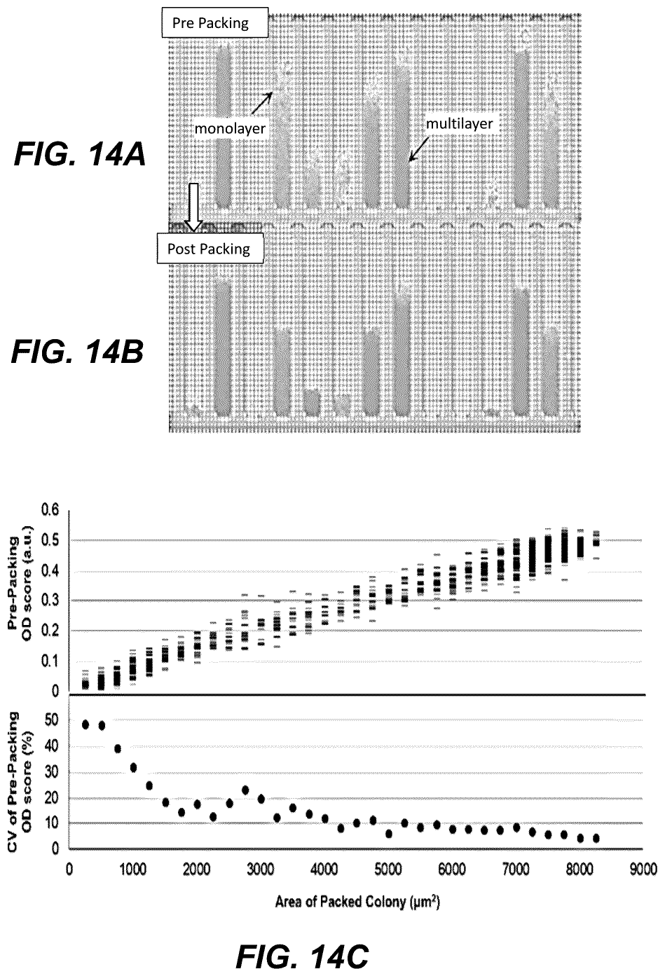

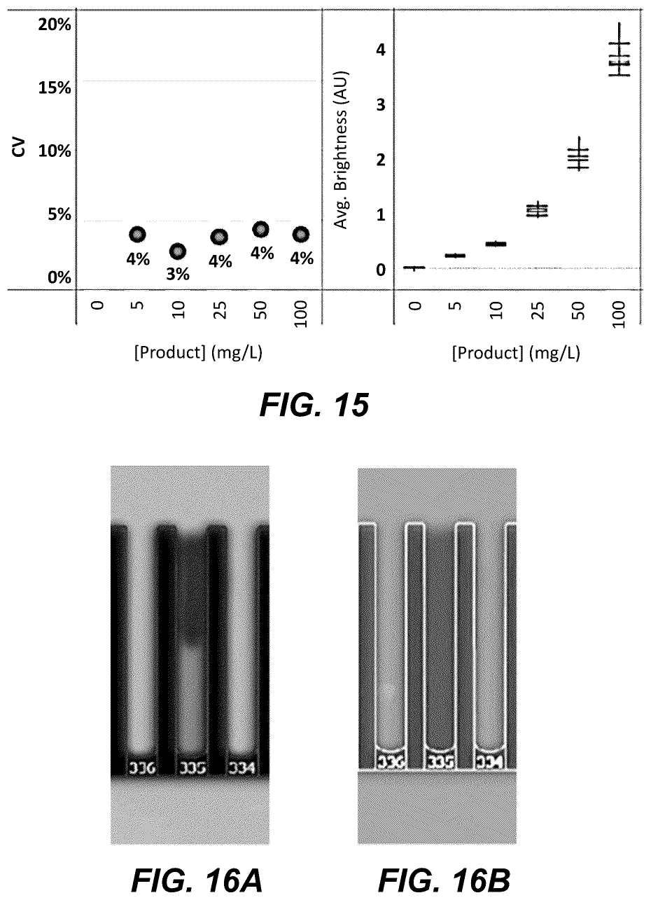

While work has been performed on secretion of macromolecules, with sufficiently slow diffusion to allow accumulation and visualization using fluorescent readouts for low-molecular-weight compounds (<1 kDa) as investigated here, rapid diffusion from the chambers was initially a concern. A method was therefore devised to seal the NanoPen chambers using a hydrophobic oil with high respiratory gas solubility, but low product and carbohydrate solubility. After sealing, each chamber effectively becomes a miniature batch reactor with fixed carbon source, accumulating product, and continuous gas exchange. When the assay is complete, the oil can again be replaced with aqueous media to allow export of desired strains (See FIGS. 14A-C and 15).

In some variations, the method may further include subsequently flowing a second aqueous medium into the channel, thereby displacing the water immiscible fluidic medium from the channel. In other variations, the method may further include flowing a third aqueous medium comprising a surfactant into the channel, thereby clearing a residual portion of the water immiscible medium from the channel. In some variations, the method may further include unpenning a selected clonal population of yeast cells out of the chamber and exporting the selected clonal population of yeast cells out of the microfluidic device.

In some embodiments, displacing substantially all of the first aqueous medium in the channel by the water immiscible fluidic medium may be performed without displacing the first aqueous medium in the chambers of the plurality of chambers.

Open Chamber Assay. The rapid development of fluorescence signal in the sealed-chamber assay described permits the use of an alternative method, in which chambers are left unsealed throughout the experiment. In this assay, local product concentration within each chamber is mainly governed by the rate of production by the colony and diffusion away from the colony. More details about this type of assay are described below in the section entitled General Diffusion Assay Techniques and also described in International Application Serial No. PCT/2017/027795, entitled “Methods, Systems, and Kits for In-Pen Assays”, filed on Apr. 14, 2017, published as International Application Publication WO2017/1811135; International Application Serial No. PCT/US2018/055918, entitled “Methods, Systems, and Kits for In-Pen Assays”, filed on Oct. 15, 2018, published as International Application Publication WO2019/075476; and International Application Serial No. PCT/US2021/021417, entitled “Methods, Systems, and Kits for In-Pen Assays”, filed on Mar. 9, 2020, published as International Application Publication WO2021/184458, the entirety of each of which disclosures are herein incorporated by reference for any purpose.

As a result of the effective boundary condition just outside of each NanoPen (product concentration≈0), the concentration gradient in the cell-free gradient measurement area at steady state will be proportional to the rate of secretion of the analyte. This allows for inference of the productivity in each pen in the few seconds preceding a single fluorescence image of the chip by simply extracting the slope of the linear fit of signal intensity with respect to position in the chamber. If images are acquired periodically, time-dependent productivity data for every colony may be collected throughout the experiment.

Accordingly, a method is provided for assessing a relative productivity of a detectable molecule by a population of yeast cells, in a microfluidic device having an enclosure including a channel and a plurality of chambers, each chamber of the plurality having an opening fluidically connecting the chamber to the channel, the method including: disposing a yeast cell configured to produce the detectable molecule in each chamber of the plurality of chambers; perfusing an aqueous medium through the channel; culturing the yeast cell to expand to a clonal population of yeast cells; increasing a rate of perfusing the aqueous medium for a selected first period of time thereby establishing a substantially steady state of diffusion of the detectable molecules from each chamber into the channel; imaging a signal from the detectable molecules produced by the clonal population of yeast cells in each chamber of the plurality; and determining a relative productivity for each clonal population of yeast cells.

In some embodiments, the molecule may be inherently detectable. In some other embodiments, the molecule may be detectable upon labelling with a detectable label as described herein.

In some variations, the first period of time may be about 1 min, about 2 min, about 3 min, about 4 min, about 5 min, about 7 min, about 10 min, about 20 min, or about 30 min.

In some variations, the rate of perfusing the aqueous medium may be increased from a first rate of flow to a second rate of flow by a factor of about 2 to about 10 for the selected period of time.

In some variations, the method may further include reducing the rate of perfusion to the first rate of flow for a second selected period of time; increasing the flow to the second rate of flow for the selected first period of time, thereby establishing the substantially steady state of diffusion of the detectable molecules; and imaging the signal from the detectable molecules produced by the clonal population of yeast cells at a second point in time. The second period of time may be an extended period of culturing before imaging again, and may be about 1 h, about 2 h, about 4 h, about 6 h or any value therebetween. The periods of culturing, interrupted by periods of increased flow, followed by imaging may be repeated over an extended culturing period of about 6 h, about 8 h, about 10 h, about 12 h, about 14 h, about 16 h, about 18 h about 20 h, about 24 h, about 36 h or more.

In some embodiments, determining the relative productivity may include determining a relative productivity of the clonal population when culturing in the presence of increasing levels of byproducts from the clonal population.

There are several advantages offered by the open-chamber assay: (1) the chemostat-like culture system likely provides a closer approximation of common fed-batch commercial processes, wherein nutrients are constantly provided and waste is removed; (2) low concentrations of feed source and other nutrients, which might be quickly depleted in a sealed assay, can be used in order to avoid the problem of overflow metabolism often observed in batch processes; (3) the export of desired clones is simplified and accelerated; (4) the open-chamber assay gives a more direct readout for rate of conversion of sugar into product, since secreted intermediates like ethanol may diffuse out of the pen before being reconsumed; (5) productivity data can be aggregated based on colony size at different time points, rather than requiring all data to be collected at a single time point with possibly wide-ranging OD scores.

In some embodiments, a feedstock used in the methods of the present disclosure comprises a carbon source including, but not limited to, sucrose, glucose, fructose, or a combination thereof, each of them can be at a concentration of from about 0.001% to 5%. In some embodiments, each of them can be at a concentration of about 0.001, about 0.005, about 0.01, about 0.05%, about 0.1%, about 0.5%, about 1%, about 1.5%, about 2%, about 2.5%, about 3%, about 3.5%, about 4%, about 4.5%, about 5%, or any range defined by two of the foregoing endpoints. In some embodiments, the carbon source is from about 0.001% to 10% in total.

In an example described in Example 3-4, differing media compositions and other factors can be varied to probe efficiency and resiliency for clones tested in the open chamber microfluidic assay. Cane syrup largely comprising sucrose is a common feedstock for commercial-scale fermentation, and thus sucrose is frequently used for microtiter plate screens. Glucose, sucrose, and an equimolar mixture of glucose and fructose were tested at 30 g/L total sugar loading in three separate screens. Similar correlations between Beacon qₚ score and bioreactor yield, volumetric productivity, and qₚ were observed for all three conditions, with the minimum within-strain variability occurring at assay times between 12 and 20 hours.

As described in detail below, the same carbon sources were also tested at 0.1% g/L. Such conditions are interesting candidates for a culture model because in feedback-regulated and exponentially fed bioreactors, steady-state extracellular sugar concentrations are typically low, a condition that is difficult to sustain in microtiter plate cultures. Growth under carbon limitation required an extended culture period to accumulate sufficient biomass for reliable normalization (for instance, >60 hours), and ultimately yielded poor correlation between Beacon qₚ score and bioreactor qₚ. Interestingly, analysis of normalized score as a function of colony size indicated a decline in the per-cell productivity in larger colonies under these conditions, which could suggest intra-colony differences in cellular access to sugar.

According to Example 3-4, low sugar culture conditions generally have worse chip-to-bioreactor correlation among the tested conditions, lower stability in productivity over time, lower dynamic range, and larger assay CV. The 1.5% glucose+1.5% fructose was therefore selected to be the carbon source for on-chip production.

Two Tiered Screening method. In light of the foregoing, a two tiered screening method is provided. In the first tier, one or more libraries may be screened per each clone, screening thousands of individual clones. After assay, selection of high scoring clones and export of those clones, the hit clones were expanded in a 96 well plate. The expanded hit population was re-submitted to the open chamber assay, where larger numbers of replicates were assayed (n>50, for example). With the higher number of replicates, strains may be selected for promotion to bioreactor expansion with higher confidence. As discussed in Example 3-5, a screening workflow timeline schematic was demonstrated that permits two dual Tier 1/Tier 2 screenings to be performed within two weeks, requiring minimal personnel effort when conducted within the automated microfluidic device and system described herein.

Each Tier-2 screen identified at least one mutant with mean qₚ score improved over the parent strain by >20%. Seven mutants improved by varying extents were then tested alongside their parent strains in Ambr250 bioreactors to assess performance at lab scale. Four of the seven strains exhibited peak bioreactor qₚ values improved over parent by 10-85%. These gains were aligned with or even larger than the magnitude of increases observed in the nanoliter culture model. The strain with the largest bioreactor qₚ improvement additionally achieved a 20% increase in average P over a 6-day fermentation, making it a lead candidate for further engineering.

These methods, performed within microfluidic systems, represent an extremely promising opportunity for the high-throughput screening of microbial cell factories. Remarkably, the methods can identify strains with improved qₚ at a volume 109-fold smaller than the bioreactor it models. Strain productivity on the microfluidic system correlated with peak bioreactor performance parameters as well as or better than data from conventional microplate culture models. The improvement in data quality may be partly attributed to the direct readout for qₚ, as opposed to endpoint yield measurements from carbon-exhausted cultures or error-prone plate-based productivity measurements. It is likely also a consequence of maintaining a roughly constant chemical environment, in which strains are held in a stable metabolic state throughout the experiment.

Moreover, the two-tiered libraries above were processed without microplates, agar trays, liquid handling robotics, or extraction solvents. In some embodiment, complete results for both tiers can be generated in under ten days-approximately half of the time typically used for a microplate library screen of comparable size with sophisticated automation. The importance of reducing this cycle time cannot be overstated, as each round of strain design in an engineering campaign depends on results from the previous round. Together, the rapid data turnaround and high data quality showcased here could reduce time-to-market for a metabolic engineering product by months or more.

As stated above, strain improvement efforts are motivated by the need to reduce manufacturing costs, which are influenced by several factors. The assays of the present disclosure could be used to improve other phenotypes as well. For example, screens could be designed to find strains more likely to sustain peak qₚ throughout a longer fermentation by (1) choosing strains with less selective pressure for mutation, (2) identifying strains with resistance to feedback inhibition by products or intermediates, or (3) uncoupling growth rate from metabolic pathway flux.

Some strains with highly improved bioreactor qₚ values exhibit relatively smaller improvements in P and Y, which must be increased to reduce manufacturing cost. The assays of the present disclosure offer the exciting opportunity to monitor qₚ and μ simultaneously, which can in theory allow one to screen for strains with improved P and Y as well. These variables can be written as:

$$P = {q}_{p} {c}_{X}$$

$$Y = \frac{{q}_{p}}{{q}_{s}} = \frac{{q}_{p}}{a μ + b {q}_{p} + c {q}_{{CO}_{2}} + …}$$

where c_{X} is the biomass density, qₛ is the specific rate of sugar consumption, q_{CO}_{₂} is the specific rate of CO₂ release, a, b, and c are stoichiometric coefficients, and “ . . . ” indicates any other byproducts formed. Because c_{X} is a direct consequence of u and dilution rate, these relationships show that in addition to qₚ, μ is also an important determinant of both P and Y.

This study has established the power of these methods as performed within microfluidic systems for predictive modeling of microbial molecule fermentations and its utility to other small-molecule targets. The low-volume chip offers flexibility by enabling assays that require precious reagents or dynamic control of the cellular environment.

### III. BIOMASS MEASUREMENT

While some cells are enough to count explicitly via digital image processing, smaller cells (e.g., microbial cells) can grow in multilayers within the chambers of the microfluidic device so that counting might not be suitable. Therefore, methods for biomass measurement in a microfluidic device are provided. The method comprises obtaining a microfluidic device comprising a chamber having a biomass to be measured, wherein the microfluidic device comprises a microfluidic circuit comprising a flow region and a chamber fluidically connected to the flow region, wherein the chamber comprises an opening to the flow region; obtaining a first brightfield image of the chamber or a first area thereof comprising the biomass; and measuring a first optical density score (OD score) from the first brightfield image.

The “OD score” measured on chip as described herein refers to a metric that is derived from the intensity of brightness measured from two bright-field images. The metric compares the brightness of a region containing the biomass in the first bright-field image, and provides a normalization from an image measured of the same region in a reference image having no biomass present. More details about the measurement and calculation of the OD score are described in the following paragraphs.

In some embodiments, the first optical density score corresponds to the measured biomass. In some embodiments, the OD score is linear/proportional to the biomass of the culture with a coefficient of variation (CV) less than 50%, 45%, 40%, 35%, 30%, 25%, 20%, 15%, 10%, 5%, or 1%. In some embodiment, the OD score is particularly representative to the biomass while colonies of larger size are measured. For instance, the colony to be measured occupies an area of larger than 1000, 1500, 2000, 2500, 3000, 3500, 4000, 4500, 5000, 5500, 6000, 6500, 7000, 7500, 8000, 8500, or 9000 um². In some embodiments, the OD score corresponds to the biomass of the culture provided that the OD score is at least 0.08, or between any two of the values: 0.08, 0.1, 0.15 0.2, 0.25, 0.3, 0.35, 0.4, 0.45, 0.5, 0.55, and 0.6.

In some embodiments, the method further comprises concentrating biomass to obtain a consolidated area containing the biomass, and wherein the first area is within the consolidated area. In certain embodiments, concentrating the biomass is performed by centrifugation or gravity.

In some embodiments, the microfluidic device is as described herein. In some embodiments, the flow region comprises a microfluidic channel, and the opening of the chamber is proximal to the microfluidic channel and oriented substantially parallel to a flow of a fluidic medium in the microfluidic channel, when the fluidic medium is flowing in the microfluidic channel. In certain embodiments, the chamber comprises an isolation region and a connection region fluidically connecting the isolation region to the flow region; and wherein the connection region comprises the opening to the flow region.

In some embodiments, obtaining the microfluidic device comprising the biomass to be measured comprises disposing a cell into the chamber, and optionally, expanding the cell into a clonal population within the chamber. In certain embodiments, disposing a cell into the chamber comprises: introducing a fluidic medium comprising the cell and penning the cell into the chamber.

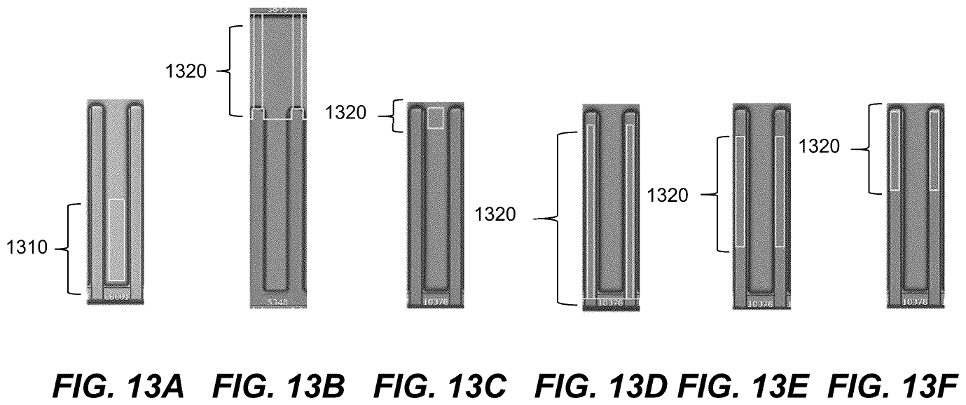

Area of interest. In some embodiments, an area of interest for measuring the biomass can be a whole area of the chamber or an area of the chamber as long as the area comprises at least a portion of the biomass to be measured. In some embodiments, the first area is within the isolation region 1310 (See FIG. 13A. In some embodiments, the first area can comprise a portion of the connection region.

Correction area. The method further comprises selecting a second area in the microfluidic circuit, wherein the second area does not comprise the biomass; measuring a second optical density within the second selected area; and correcting the first optical density with the second optical density. The second area (i.e., a correction area) can be with an area 1320 of the microfluidic channel (FIG. 13B, an area 1320 of the connection region (i.e., a sweeping region of the chamber. FIG. 13C, an area 1320 of the walls of the chamber (FIG. 13D, a portion of the walls 1320 (FIG. 13E and FIG. 13F. In some embodiments, the correction area can be within an empty (i.e., without any biomass) chamber of the same microfluidic device having the chamber comprising the biomass to be measured.

In some embodiments, the second optical density is measured from a second brightfield image. In certain embodiments, the second brightfield image is taken under substantially same illumination conditions as the first brightfield image. As used herein, “substantially same illumination conditions” refers to there is no difference or the difference between the illumination conditions under which the two or more bright-field images were taken can be ignored. In some embodiments, “substantially same illumination conditions” refers to the difference between the illumination conditions under which the two or more bright-field images were taken has been normalized so that it would not significantly affect the results of the assays. The parameters to be considered in terms of “substantially same illumination conditions” might include, but not limited to, illumination wavelength and/or power, exposure time, magnification, field of view, angle of view, or a combination thereof. In some embodiments, the second optical density is measured from the first brightfield image.

Normalization and Correction. In some embodiments, the first optical density score can be normalized by a reference optical density score. The reference optical density score can be obtained from a reference brightfield image, which is taken when the chamber is empty (i.e., without any cells/biomass). In certain embodiments, the reference brightfield image is taken before the cell is introduced into the microfluidic device. In some embodiments, the reference optical density score is measured in an area the same as the first area for the first optical density score. In certain embodiments, the reference brightfield image is taken under substantially same illumination conditions as the first brightfield image.

In some embodiments, the OD score can be normalized by the following formula of standard OD:

$${OD}_{i , t} = 1 - \frac{{\overline{I}}_{i , t}}{{\overline{I}}_{i , Ref}}$$

where OD_{i,t} is the score for pen i at time t; Ī_{i,t} is the corrected brightness of pen i at time t; and Ī_{i,Ref} is the corrected brightness of pen i in a reference image. Ī_{i,t} and Ī_{i,Ref} are defined as:

$${\overline{I}}_{i , t} = \frac{{I}_{i , t}}{\frac{1}{\vert {E}_{v} \vert} {\sum }_{x ∈ {E}_{v}} {I}_{x , t}}$$

$${\overline{I}}_{i , Ref} = \frac{{I}_{i , Ref}}{\frac{1}{\vert {E}_{v} \vert} {\sum }_{x ∈ {E}_{v}} {I}_{x , Ref}}$$

where Eᵥ is the set of empty pens in image v, and the denominators represent the average raw brightness of empty pens.

Alternatively, the OD score can be obtained by the following formula of logarithmic OD:

$$OD = {Log}_{10} ( \frac{{I}_{pen - {t}_{e}}}{{I}_{pen - {t}_{i}}} ) * ( \frac{10000}{2 * Thickness} )$$

$${I}_{pen} = \frac{{I}_{C} - k * {I}_{R}}{{I}_{R} * ( 1 - k )}$$

where “Thickness” refers to the thickness of the chip, which, in some embodiments, is about 35 microns. I_{C} is the intensity measured within the area of interest (cell counting area); I_{R} is the intensity measured within the reference area. Factor k accounts for non-specific reflection of lights of the chip's glass-air and glass-air interface, which, in some embodiments, is estimated to be 0.2 accounting for 20% of non-specific reflection.

To validate OD score as a biomass measurement, chambers in the microfluidic device were seeded with varying numbers of S. cerevisiae cells from a mixture of strains, which were then cultured on the Beacon system until a range of colony sizes was visible. After imaging the chip (FIG. 14A) and obtaining a pre-packing OD score (see top panel of FIG. 14C), cells were packed tightly into the NanoPen chambers via centrifugation to achieve uniform density and then reimaged (FIG. 14B). The area, e.g., consolidated area, of the resulting packed colony was assumed to be a linear reflection of biomass. The OD score was linear over a wide range of colony sizes (upper panel of FIG. 14C), with typical CV<15% for colonies >3500 um² in area, e.g., having an OD score 0.15-0.30. When the area occupied by cells was too small, e.g., less than about 2000 cubic microns, the CV increased markedly (lower panel of FIG. 14C). Additionally, unpacked colony area did not linearly predict packed colony area, likely because larger colonies accumulated biomass in multilayers rather than strictly by increasing in area.

The OD score can be used to normalize the productivity measurement as described above. For example, in some embodiments, the productivity at pixel i at time 1 (C_{i,t}) of a sealed chamber assay or an open chamber assay can be calculated as below.

$${C}_{i , t} = \frac{{F}_{i , t} - {F}_{i , Bg}}{{F}_{i , Ref} - {F}_{i , Bg}}$$

where F_{i,t} is the fluorescence intensity at pixel i at given time t; F_{i,Bg} is the fluorescence intensity of the background reference at pixel i; and F_{i,Ref} is the fluorescence intensity of the 50 mg/L reference at pixel i. In the Sealed-Pen Assay, the total relative productivity was quantified by measuring the increase of product concentration in each NanoPen divided by the assay duration. In the open-pen assay, the total relative productivity was quantified by measuring the gradient of product concentration 20 μm below the opening of each NanoPen. The qₚ score of each colony was then calculated as the total relative productivity divided by OD score.

Biomass measurement error. In some embodiments, to maximize assay resolving power, colonies were therefore pre-filtered before analysis, including only those with an OD score above a threshold value of 0.025, 0.05, or 0.08. As above, colony area showed poorer linearity with post-packing area, and was not a reliable measurement of biomass.

Chip image analysis. In a library screen workflow, the top producers were selected based on assay scores with customizable criteria. For instance, the exported colonies may be selected to be colonies which maintained the highest qₚ scores over 3 consecutive assay time points, equivalent to a 3-hour duration.

Statistical analysis. Cell metabolic activity is known to vary among cells with a common genotype and fluctuate stochastically in time within single cells. For colonies containing small numbers of cells, such heterogeneity can contribute meaningfully to variation in measurements made on samples of a single strain. In some embodiments, screening efforts seek to identify strains with improved ensemble performance in a bioreactor; therefore, the summary statistic of interest for distinguishing strain performance in Tier-2 screens was the 95% confidence interval of the mean qₚ score, which is represented by error bars in plots of Tier-2 data. Additional information on sources of variability is discussed as follows 12.

Assessment of measurement error and pen-to-pen phenotypic heterogeneity. Variability in measurements of biomass and productivity among colonies of a single genotype can have a number of contributing factors, including: biomass measurement error (as discussed above); fluorescence measurement error; deviation from theoretical steady-state diffusion model, which is applicable to open assay only; intraclonal phenotypic heterogeneity (pen-to-pen, within the same genotype); process-dominated (e.g., nutrient or temperature gradients); biological (e.g., epigenetic); intra-colony phenotypic heterogeneity (e.g., spatial condition inhomogeneity); and/or single-cell phenotypic stochasticity (temporal; evident only at very low cell count).

Fluorescence measurement error. Error in measurement of average fluorescence for a single chamber in the sealed-pen assay was estimated by equilibrating an entire microfluidic chip with the analyte at multiple concentrations and comparing the assay values across the chip. CV values were observed to be <5% at all relevant concentrations (FIG. 15).

Assuming no covariance between fluorescence and OD score, error can be estimated by propagating the errors from the two measurements taken in the assay, which are combined by division in the calculation of normalized score:

$${σ}_{{q}_{p}} \approx {μ}_{{q}_{p}} \sqrt{{( \frac{{σ}_{p r o d}}{{μ}_{p r o d}} )}^{2} + {( \frac{{σ}_{OD}}{{μ}_{O D}} )}^{2}} ,$$

where σ and μ respectively represent the standard deviation and mean value of calculated sealed-pen productivity, OD score, or qₚ score. This can be equivalently written as:

$$C {V}_{q P} \approx \sqrt{{{CV}_{p r o d}}^{2} + {{CV}_{OD}}^{2}} ,$$

where CV is the coefficient of variation (σ/μ).

The errors from the biomass and productivity measurements were estimated to have a lower bound of 5% and 15%, which gives a rough expected lower bound CV of 16% for the overall assay. This value aligns with the CV values of 15-20% observed for larger colonies of any strain, suggesting that future improvements on the assay noise could probably be achieved by reducing the noise in the biomass measurement.

To estimate residual error due to other sources besides measurement of biomass or fluorescence, the assumption can be made that such errors are additive with the assay variability:

$${σ}_{o b s} \approx \sqrt{{{σ}_{assay}}^{2} + {{σ}_{residual}}^{2}}$$

To assess sources of residual error (σ_{residual} up to ˜12% of the mean), tests were performed on several strains for regional bias within an OptoSelect chip. Even under carbon limitation conditions, no obvious spatial bias in the qₚ score was observed at any perfusion rate, indicating that media exchange was fast relative to the rate of glucose uptake.

Only one example of bias was observed during this work, manifested as an edge effect in the oil sealed-pen assay. This was likely a result of differential access to oxygen, which can easily permeate the walls within the microfluidic chamber, but not the top or bottom electrodes of the chip. It was possible to remove the effect by periodically perfusing air bubbles through the main channel, which ensured an excess of oxygen in the chip.

### IV. MICROFLUIDIC DEVICES AND SYSTEMS

It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g., rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g., photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n−1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1B, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object. In some instances, the trap 132 comprises a side passage 134 that is smaller than the target micro-object in order to facilitate flow through the trap 132.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

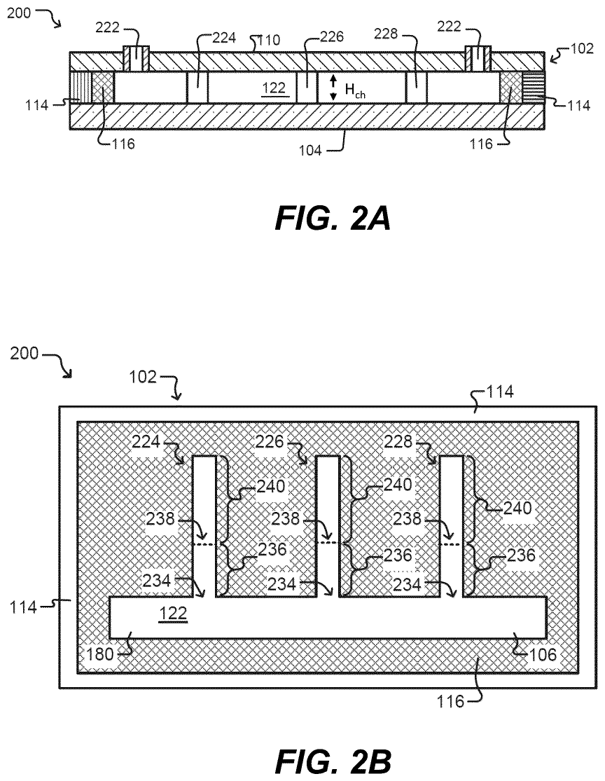

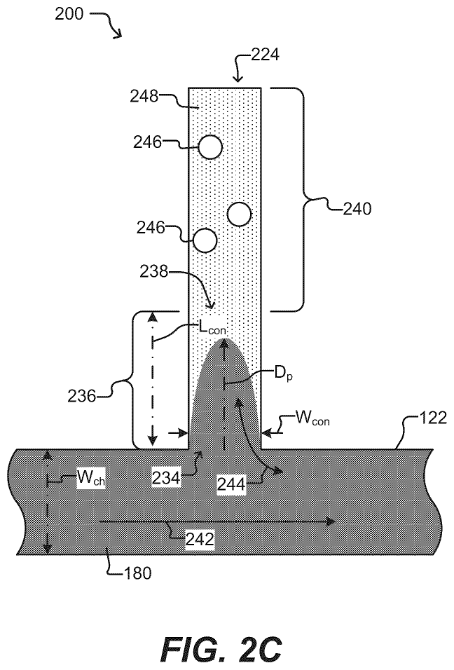

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%, 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%. 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width W_{ch} of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94%95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g., a portion of the connection region adjacent to the proximal opening 234).

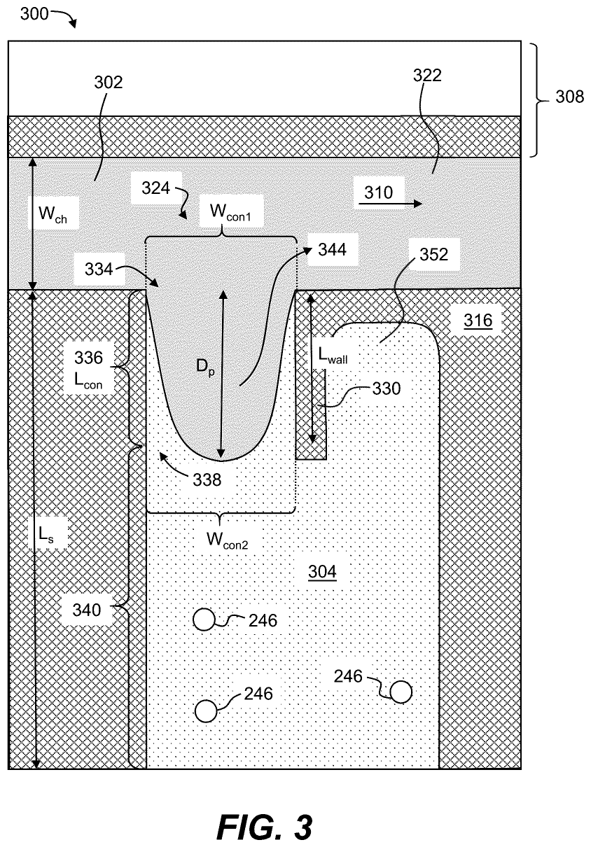

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 include a microfluidic channel 322, having a width W_{ch}, as described herein, and containing a flow 310 of first fluidic medium 302 and one or more sequestration pens 324 (only one illustrated in FIG. 3). The sequestration pens 324 each have a length Lₛ, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n−1 openings can be valved. When the n−1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Microfluidic circuit element dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure.

For any of the microfluidic devices described herein, a microfluidic channel may have a uniform cross sectional height along its length that is a substantially uniform cross sectional height, and may be any cross sectional height as described herein. At any point along the microfluidic channel, the substantially uniform cross sectional height of the channel, the upper surface of which is defined by the inner surface of the cover and the lower surface of which is defined by the inner surface of the base, may be substantially the same as the cross sectional height at any other point along the channel, e.g., having a cross sectional height that is no more than about 10%, about 9%, about 8%, about 7%, about 6%, about 5%, about 4%, about 3%, about 2% or about 1% or less, different from the cross-sectional height of any other location within the channel.

Additionally, the chamber(s), e.g., sequestration pen(s), of the microfluidic devices described herein, may be disposed substantially in a coplanar orientation relative to the microfluidic channel into which the chamber(s) open. That is, the enclosed volume of the chamber(s) is formed by an upper surface that is defined by the inner surface of the cover, a lower surface defined by the inner surface of the base, and walls defined by the microfluidic circuit material. Therefore, the lower surface of the chamber(s) may be coplanar to the lower surface of the microfluidic channel, e.g., substantially coplanar. The upper surface of the chamber may be coplanar to the upper surface of the microfluidic channel, e.g., substantially coplanar. Accordingly, the chamber(s) may have a cross-sectional height, which may have any values as described herein, that is the same as the channel, e.g., substantially the same, and the chamber(s) and microfluidic channel(s) within the microfluidic device may have a substantially uniform cross sectional height throughout the flow region of the microfluidic device, and may be substantially coplanar throughout the microfluidic device.

Coplanarity of the lower surfaces of the chamber(s) and the microfluidic channel(s) can offer distinct advantage with repositioning micro-objects within the microfluidic device using DEP or magnetic force. Penning and unpenning of micro-objects, and in particular selective penning/selective unpenning, can be greatly facilitated when the lower surfaces of the chamber(s) and the microfluidic channel to which the chamber(s) open have a coplanar orientation.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or W_{con1}) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above. Generally, however, the width (W_{con} or W_{con1}) of the proximal opening of the connection region of a sequestration pen is less than the width (W_{ch}) of the microfluidic channel. In some embodiments, the width (W_{con} or W_{con1}) of the proximal opening is about 8%, 9%, 10%, 11%, 12%, 13%, 14%, 15%, 16%, 17%, 18%, 19%, 20%, 22%, 24%, 25%, or 30% of the width (W_{ch}) of the microfluidic channel. That is, the width (W_{ch}) of the microfluidic channel may be at least 2.5 times, 3.0 times, 3.5 times, 4.0 times, 4.5 times, 5.0 times, 6.0 times, 7.0 times, 8.0 times, 9.0 times or at least 10.0 times the width (W_{con} or W_{con1}) of the proximal opening of the connection region of the sequestration pen.

In some embodiments, the size W_{C} (e.g., cross-sectional width W_{ch}, diameter, area, or the like) of the channel 122, 322, 618, 718 can be about one and a quarter (1.25), about one and a half (1.5), about two, about two and a half (2.5), about three (3), or more times the size W_{O} (e.g., cross-sectional width W_{con}, diameter, area, or the like) of a chamber opening, e.g., sequestration pen opening 234, 334, and the like. This can reduce the extent of secondary flow and the rate of diffusion (or diffusion flux) through the opening 234, 334 for materials diffusing from a selected chamber (e.g., like sequestration pens 224, 226 of FIG. 2B) into channel 122, 322, 618, 718 and subsequently re-entering a downstream or adjacent chamber (e.g., like sequestration pen 228). The rate of diffusion of a molecule (e.g., an analyte of interest, such as an antibody) is dependent on a number of factors, including (without limitation) temperature, viscosity of the medium, and the coefficient of diffusion Do of the molecule. For example, the Do for an IgG antibody in aqueous solution at about 20° C. is about 4.4×10⁻⁷ cm²/sec, while the kinematic viscosity of cell culture medium is about 9×10⁻⁴ m²/sec. Thus, an antibody in cell culture medium at about 20° C. can have a rate of diffusion of about 0.5 microns/sec. Accordingly, in some embodiments, a time period for diffusion from a biological micro-object located within a sequestration pen such as 224, 226, 228, 324 into the channel 122, 322, 618, 718 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37° C.) or decreased (e.g., to about 15° C., 10° C., or 4° C.) thereby increasing or decreasing the rate of diffusion, respectively. Alternatively, or in addition, the concentrations of solutes in the medium can be increased or decreased as discussed herein to isolate a selected pen from solutes from other upstream pens.

Accordingly, in some variations, the width (e.g., W_{ch}) of the microfluidic channel at the proximal opening to the connection region of a sequestration pen may be about 50 to 500 microns, about 50 to 300 microns, about 50 to 200 microns, about 70 to 500 microns, about to 70-300 microns, about 70 to 250 microns, about 70 to 200 microns, about 70 to 150 microns, about 70 to 100 microns, about 80 to 500 microns, about 80 to 300 microns, about 80 to 250 microns, about 80 to 200 microns, about 80 to 150 microns, about 90 to 500 microns, about 90 to 300 microns, about 90 to 250 microns, about 90 to 200 microns, about 90 to 150 microns, about 100 to 500 microns, about 100 to 300 microns, about 100 to 250 microns, about 100 to 200 microns, or about 100 to 150 microns. In some embodiments, the width W_{ch} of the microfluidic channel at the proximal opening to the connection region of a sequestration pen may be about 70 to 250 microns, about 80 to 200 microns, or about 90 to 150 microns. The width W_{con} of the opening of the chamber (e.g., sequestration pen) may be about 20 to 100 microns; about 30 to 90 microns; or about 20 to 60 microns. In some embodiments, W_{ch} is about 70-250 microns and W_{con} is about 20 to 100 microns; W_{ch} is about 80 to 200 microns and W_{con} is about 30 to 90 microns; W_{ch} is about 90 to 150 microns, and W_{con} is about 20 to 60 microns; or any combination of the widths of W_{ch} and W_{con} thereof.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W_{con1} of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Lₛ of about 40-600 microns, about 40-500 microns, about 40-400 microns, about 40-300 microns, about 40-200 microns, about 40-100 microns or about 40-80 microns. The foregoing are examples only, and a sequestration pen may have a length Lₛ selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, 20, 22, 24, or 25 microliters/sec. The foregoing are examples only, and the flow of fluidic medium within the microfluidic channel can have a maximum velocity (e.g., Vₘₐₓ) selected to be a value between any of the values listed above. The flow of fluidic medium within the microfluidic channel typically may be flowed at a rate less than the Vₘₐₓ. While the Vₘₐₓ may vary depending on the specific size and numbers of channel and sequestration pens opening thereto, a fluidic medium may be flowed at about 0.1 microliters/sec to about 20 microliters/sec; about 0.1 microliters/sec to about 15 microliters/sec; about 0.1 microliters/sec to about 12 microliters/sec, about 0.1 microliters/sec to about 10 microliters/sec; about 0.1 microliter/see to about 7 microliters/sec without exceeding the Vₘₐₓ. In some portions of a typical workflow, a flow rate of a fluidic medium may be about 0.1 microliters/sec; about 0.5 microliters/sec; about 1.0 microliters/sec; about 2.0 microliters/sec; about 3.0 microliters/sec; about 4.0 microliters/sec; about 5.0 microliters/sec; about 6.0 microliters/sec; about 7.0 microliters/sec; about 8.0 microliters/sec; about 9.0 microliters/sec; about 10.0 microliters/sec; about 11.0 microliters/sec; or any range defined by two of the foregoing values, e.g., 1-5 microliters/sec or 5-10 microliters/sec. The flow rate of a fluidic medium in the microfluidic channel may be equal to or less than about 12 microliters/sec; about 10 microliters/sec; about 8 microliters/sec, or about 6 microliters/sec.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG M_{w}<100,000 Da) or alternatively polyethylene oxide (PEO, M_{w}>100,000). In some embodiments, a PEG may have an M_{w} of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules: second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g., vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

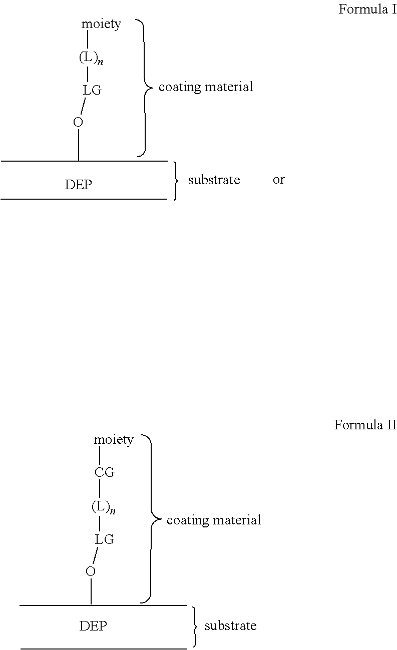

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rx and a reactive pairing moiety Rₚₓ (i.e., a moiety configured to react with the reactive moiety Rx). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology(ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Pat. No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Pat. No. 9,533,306 (Chiou, et al.), each of which is incorporated herein by reference in its entirety. Examples of suitable optically-actuated electrokinetic devices that include combined OET/OEW configured devices can include those illustrated in U.S. Patent Application Publication No. 2015/0306598 (Khandros, et al.), U.S. Patent Application Publication No 2015/0306599 (Khandros, et al.), and U.S. Patent Application Publication No. 2017/0173580 (Lowe, et al.), each of which is incorporated herein by reference in its entirety.

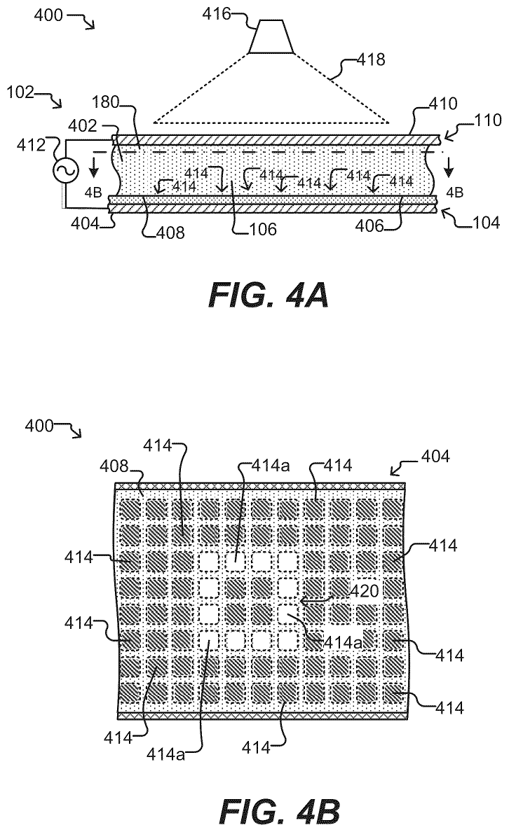

It should be understood that, for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 400 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 414 can be created anywhere and in any pattern on the inner surface 408 of the electrode activation substrate 406, in accordance with the light pattern 418. The number and pattern of the DEP electrode regions 414 thus need not be fixed, but can correspond to the light pattern 418. Examples of microfluidic devices having a DEP configuration comprising a photoconductive layer such as discussed above have been described, for example, in U.S. Pat. No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 402, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-object (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrate that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source 192 referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 202, as also discussed above. Such frequency ranges and average or peak power ranges are known in the art. See, e.g., U.S. Pat. No. 6,958,132 (Chiou, et al.), US Pat. No. RE44,711 (Wu, et al.) (originally issued as U.S. Pat. No. 7,612,355), and U.S. Patent Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz, et al.), which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied in an alternating manner with the other forces. For the microfluidic devices described herein, repositioning of micro-objects may not generally rely upon gravity or hydrodynamic forces to position or trap micro-objects at a selected position. Gravity may be chosen as one form of repositioning force, but the ability to reposition of micro-objects within the microfluidic device does not rely solely upon the use of gravity. While fluid flow in the microfluidic channels may be used to introduce micro-objects into the microfluidic channels (e.g., flow region), such regional flow is not relied upon to pen or unpen micro-objects, while localized flow (e.g., force derived from actuating a deformable surface) may, in some embodiments, be selected from amongst the other types of repositioning forces described herein to pen or unpen micro-objects or to export them from the microfluidic device.

When DEP is used to reposition micro-objects, bulk fluidic flow in a channel is generally stopped prior to applying DEP to micro-objects to reposition the micro-objects within the microfluidic circuit of the device, whether the micro-objects are being repositioned from the channel into a sequestration pen or from a sequestration pen into the channel. Bulk fluidic flow may be resumed thereafter.

System. Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an optoelectrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g., for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g., a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e., at 0° relative to x- and y-axes), a vertical orientation (i.e., at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3°, 4°, 5°, or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e., an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long-term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

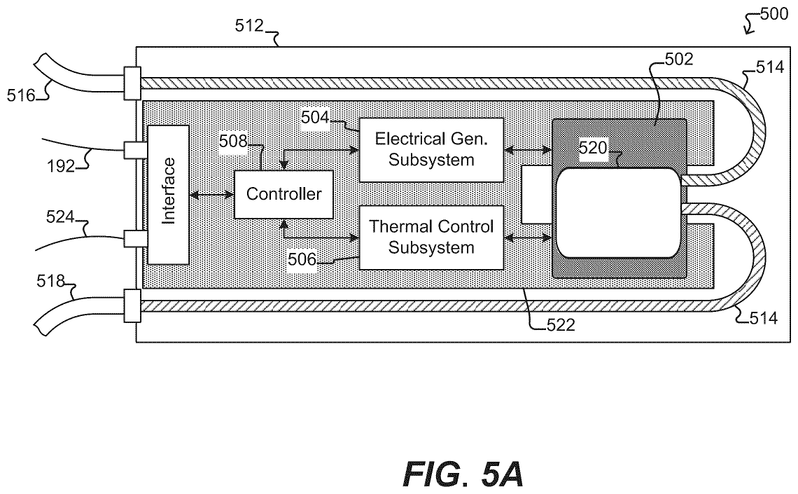

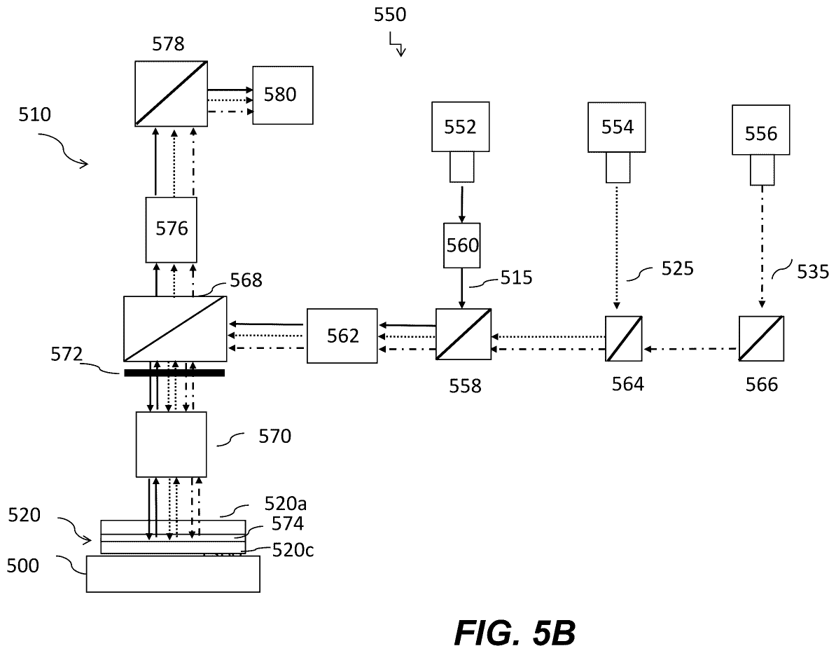

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 522, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 5A the controller 508 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns×5 microns to about 10 microns×10 microns, or any values therebetween. The structured light modulator 560 can include an array of mirrors (or pixels) that is 2000×1000, 2580×1600, 3000×2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 562 can have an aperture that is large enough to capture all (or substantially all) of the light beams emanating from the structured light modulator 560.

The structured light 515 having a wavelength of about 400 nm to about 710 nm, may alternatively or in addition, provide fluorescent excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive illumination light 535 from the third light source 556), and the second light, brightfield illumination light 525, may be transmitted therefrom to the first dichroic beam splitter 558. The second light, brightfield illumination light 525, may then be transmitted from the first dichroic beam splitter 558 to the first tube lens 562.

The third light source 556 can transmit light through a matched pair relay lens (not shown) to a mirror 566. The third illumination light 535 may therefrom be reflected to the second dichroic beam splitter 564 and be transmitted therefrom to the first beam splitter 558, and onward to the first tube lens 562. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 556) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 510. The objective lens 570 can have one or more magnification levels available such as, 4X, 10X, 20X.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 510 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattern, into each of the plurality of sequestration pens in the sample plane 574 within the field of view. For example, each of the plurality of illumination spots can have a size of about 5 microns×5 microns; 10 microns×10 microns; 10 microns×30 microns, 30 microns×60 microns, 40 microns×40 microns, 40 microns×60 microns, 60 microns×120 microns, 80 microns×100 microns, 100 microns×140 microns and any values there between. The illumination spots may individually have a shape that is circular, square, or rectangular. Alternatively, the illumination spots may be grouped within a plurality of illumination spots (e.g., an illumination pattern) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. No. 8,921,055 (Chapman), U.S. Pat. No. 10,010,882 (White et al.), and U.S. Pat. No. 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Additional system components for maintenance of viability of cells within the sequestration pens of the microfluidic device. In order to promote growth and/or expansion of cell populations, environmental conditions conducive to maintaining functional cells may be provided by additional components of the system. For example, such additional components can provide nutrients, cell growth signaling species, pH modulation, gas exchange, temperature control, and removal of waste products from cells.

### V. EXAMPLES

System and device: An OptoSelect™ device, a nanofluidic device controlled by an optical instrument, BeaconR were employed (Both are manufactured by Berkeley Lights, Inc.) The instrument includes: a mounting stage for the chip coupled to a temperature controller; a pump and fluid medium conditioning component; and an optical train including a camera and a structured light source suitable for activating phototransistors within the chip. The OptoSelect device includes a substrate configured with OptoElectroPositioning (OEP™) technology, which provides a phototransistor-activated OEP force. The chip also included a plurality of microfluidic channels, each having a plurality of NanoPen™ chambers (or chambers) fluidically connected thereto. The volume of each chamber is around 1×10⁶ cubic microns.

Device priming. 250 microliters of 100% carbon dioxide were flowed in to the OptoSelect device at a rate of 12 microliters/sec, followed by 250 microliters of PBS containing 0.1% PluronicR F27 (Life Technologies® Cat #P6866) flowed in at 12 microliters/sec, and finally 250 microliters of PBS flowed in at 12 microliters/sec. Introduction of a Wetting Solution followed, which introduces a conditioned surface to the surfaces within the microfluidic device. The details of the surface and its introduction are described in US Application Publication US 2016/0312165, filed on Apr. 22, 2016 and U.S. Application Publication No. US2019/0275516, filed on Nov. 20, 2018, each of which disclosures are herein incorporated by reference in its entirety.

Media perfusion during culture. Medium is perfused through the OptoSelect™ device according to either of the following two methods: (1) Perfuse at 0.01 microliters/sec for 2 h; perfuse at 2 microliters/sec for 64 sec; and repeat. (2) Perfuse at 0.02 microliters/sec for 100 sec; stop flow 500 sec; perfuse at 2 microliters/sec for 64 sec; and repeat.

System Preparation. Prior to initiating the workflow, the Beacon instrument was sterilized by purging all fluidic lines with SporGon decontamination reagent (Decon Labs, Inc.). After soaking for 2 hours, lines were rinsed with sterile water for 1 hour to eliminate residual SporGon.

Following sterilization, OptoSelect™ chips were loaded onto Beacon instrument for a sequence of pre-workflow operations. The Wetting Solution was introduced to the chip and then incubated to functionalize the surfaces. DI water was then flushed through each chip to remove the Wetting Solution. To enable the sealed-pen productivity assay (see section below), a specialized differential wetting procedure was implemented in place of the typical wetting procedure. In this case, a functionalization that renders surfaces hydrophobic was implemented to wet the surface along the channels exclusively, while the standard Wetting Solution was used to wet the surface in the NanoPen chambers, e.g., sequestration pen(s). Differential wetting allowed for different surface properties between the channels and the NanoPen chambers, enhancing the sealing performance in the sealed-pen productivity assay.

After wetting and priming, the Beacon instrument automatically located the fiducial markers on the chips for x-y stage and focus calibration. Lastly, two types of reference imaging were performed sequentially. The bright-field high-magnification (10× objective) images were taken across the chips as a reference for the quantification of colony size during cell culture. Subsequently, the fluorescence images (4× objective, FITC channel) were captured before and after equilibrating 50 mg/L of the final product throughout all OptoSelect chips. The fluorescence images taken before and after equilibration were used as the background reference and the normalization reference, respectively, for the quantification of product concentration during the productivity assay.

Single-Cell Loading. Broth from microplate precultures or mutagenesis recovery was diluted in PBS to a target OD600 of 0.1 and transferred to a 300-uL 96-well plate for import (Corning). The import plate was then loaded into the well-plate incubator on the Beacon instrument for single cell loading. During the loading process, the temperatures of the well-plate incubator and the OptoSelect chips were set at 4° C. and 18° C., respectively, to reduce cell proliferation during the loading period.

For each strain to be loaded into the NanoPen chambers, 25 μL of cell suspension was imported from the well-plate incubator to the channels of the OptoSelect chips. Single cells were identified automatically by the Beacon instrument control software using a convolutional neural network algorithm optimized for yeast cells. A positioning strategy was then automatically implemented to maximize loading throughput using the OEP technology. Residual cells in the channels were flushed to waste after loading. In general, it took roughly 4-5 hours to load the cells across all 4 chips in a single library screen workflow.

### Experiment 1: Biomass Measurement of Nonmammalian Cells

### Example 1-1: Yeast Culture and Standard Curve

Cells from a Pichia cell population engineered to secrete a non-endogenous product, e.g., protein, organic molecule, and the like, were suspended in a load medium (145 mM cellobiose, 0.3 mM phosphate buffer, 0.2 mM MgCl₂, 0.4 mM HEPES and 0.1% F127) and introduced into an OptoSelect™ microfluidic device, e.g., chip, in a Beacon® Optofluidic system for biomass measurement. In Examples 1-1 to 2-3, the chip is a sealed chip, which prevents exchange of gaseous components through the edges of the chip construction. The sealed chip is described in further detail in International Application Serial No. PCT/US2021/048196, entitled “Apparatuses, Methods and Kits for Microfluidic Assays”, filed on Aug. 30, 2021, and published as International Application Publication WO2022/047290, the entire disclosure of which is herein incorporated by reference. Single cells were disposed into respective sequestration pens and cultured on-chip at 27° C. with constant perfusion of fresh medium BMGY (1% glycerol) and 80% air fraction. Time-lapse bright-field images were taken every 30 minutes to keep track of cell growth for each colony. After 48 hours, the bright-field biomass measurements were analyzed using the Cell Analysis Suite 2.1 Software from Berkeley Lights (See International Application No. PCT/US2020/060784, entitled “Systems and Methods for Analysis of Biological Samples, filed on Nov. 16, 2020 and published as International Application Publication WO20210987449 A1, the entire disclosure of which is incorporated by reference for any purpose).

The OD score of the colony in each sequestration pen was quantified as described above in the section entitled “Biomass Measurement”. However, other methods of measuring OD may be used in other variations of this experiment.

The cells in each sequestration pen were also counted by using the Dalmatian algorithm as described in Sergey A Shuvaev et al., DALMATIAN: An Algorithm for Automatic Cell Detection and Counting in 3D., Front Neuroanat 2017 Dec. 12; 11:117, which disclosure is herein incorporated by reference in its entirety. The OD scores were plotted against the cell counts, and the linear correlation between the OD scores and the cell counts were determined by using a linear regression model. The coefficient of variations (CV) of cell counts according to 0.02 OD score intervals were determined respectively.

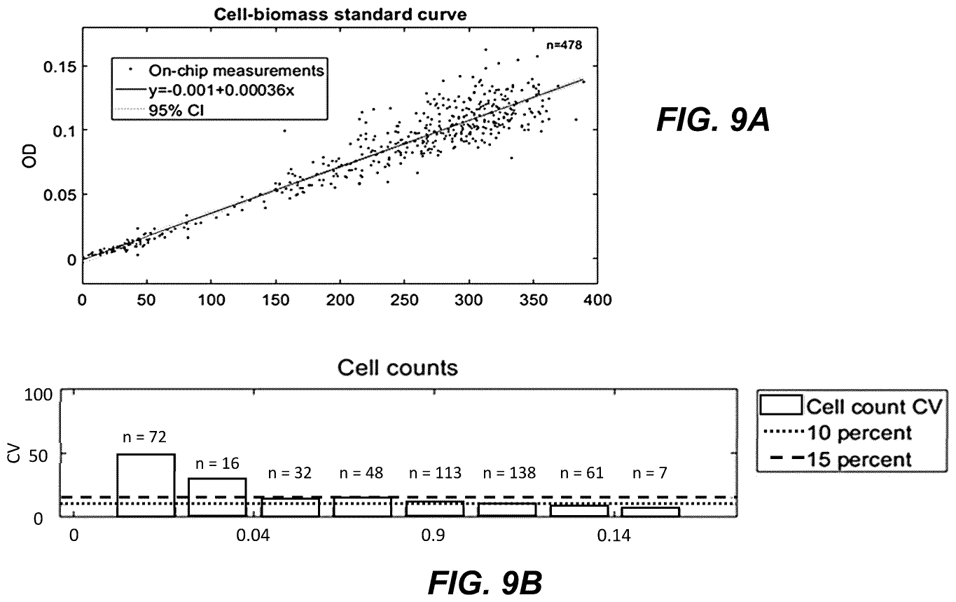

The results show that the OD scores obtained by the method of the present disclosure were correlated with the cell count number using Dalmatian algorithm (FIG. 9A). Furthermore, the CV values were below 15% by any OD scores above 0.04 and below 10% for any OD scores above around 0.09 (FIG. 9B). These results prove the method of the present disclosure can provide liable biomass measurement that is relative to actual cell counts.

### Example 1-2: Yeast Culture with Centrifugation

S. cerevisiae cells from a population of genetically modified cells were disposed at varying numbers into respective sequestration pens of an OptoSelect™ chip in a Beacon® Optofluidic system for biomass measurement. The cells were then cultured on-chip at 33.5° C. with constant perfusion of fresh media (BSM+carbon source) at 0.1 uL/s until a range of colony sizes was visible. Time-lapse bright-field images were taken every hour to keep track of cell growth for each colony. The bright-field biomass measurements were analyzed using the Cell Analysis Suite 2.1 Software, as mentioned above.

The OD score of each colony at a given time was quantified as described above. After imaging the chip, cells were packed tightly in the sequestration pen via centrifugation (1,000 g, 10 minutes) to achieve uniform density before reimaging to validate OD score as a biomass measurement for the colonies.

FIG. 14A shows the bright-field images of the sequestration pens before and after centrifugation (FIG. 14B). The area of the resulting centrifuged colony was assumed to be a linear reflection of biomass. The OD score of the colonies measured before centrifugation was proportional to this centrifuged colony area, indicating that it was linear over a wide range of colony sizes (FIG. 14C), with typical CV<15% for colonies >3500 μm² in area. Moreover, a coefficient of variation (CV) of 10-15% for the range of biomass measured in larger colonies (OD score 0.15-0.30) was observed. To maximize assay resolving power, colonies were therefore pre-filtered before analysis, including only those with an OD score above a threshold value of 0.08. Notably, colony area showed poorer linearity with post-centrifugation area, and was not a reliable measurement of biomass

### Experiment 2: Bioproductivity Assessment: Bead Assay, Diffusion Gradient Assay, and Accumulation Assay

Example 2-1: Hydrogels forming assay barriers. In Examples 2-2 through 2-4, variations on an 8 arm 20K PEG polymer were used to form the hydrogel barriers. The extent of crosslinking with resultant effects on permeability/impermeability was controlled by varying the length of photopatterning (about 1000 msec to about 5 sec, or repeated exposures of about 1000 msec); the concentration of inhibitor present in the hydrogel solution introduced into the microfluidic device; and the proportion of crosslinkable moieties on the 8 armed modified PEG polymer.

Variation A. The effect of hydrogel composition and extent of crosslinking was examined. Two polymer compositions were examined: the first as an 8 arm, 8 acrylamide terminated 20K PEG polymer. The second composition was a 90:10 mixture of the 8 arm, 8 acrylamide terminated 20K PEG polymer: linear (1 arm) acrylamide 20 kDa PEG polymer. Each was introduced to a microfluidic chip and polymerized in different sections using different lengths of exposure and power, ranging from 40% power to 100% power, and 3 sec to 20 sec. Some of these conditions did not create fully polymerized hydrogel barriers (FIG. 7A, 705 type having a thickness of about 100 microns). However, 50% power, 20 sec ensures fully sealing barriers and was used for diffusion experiments.

A mixture of FITC-labelled IgG (150 kDa) and Alexa-647 labeled streptavidin (66 kDa) was perfused through the device after hydrogel formation. Images were obtained 1 hr after initial introduction of the labelled materials in the respective color cube. As seen in FIG. 16A, the hydrogel barrier permitted a significant amount of fluorescently labeled streptavidin to diffuse into the culturing area distal to the hydrogel barrier. In contrast, in FIG. 16B, the same pen (annotated as 336) did not permit diffusion of the FITC-IgG across the hydrogel barrier. Results (data not shown) were the same with the hydrogel barrier formed from the 90:10 mixture of 8 arm: linear PEG polymers.

Variation B. Permeability control was examined by creating hydrogel barriers fully spanning the width of the sequestration pen.

The behavior of two different formulations was examined (formulation F1 equals 100%8 arm 20K PEG having 8 acrylamide termini; formulation F2 equals 25%8 arm 20K PEG having 8 acrylamide termini: 75%8 arm 20K PEG having 1 acrylamide terminus, 7 non-crosslinkable termini (e.g., hydroxyl termini). The initiator and inhibitor ratios were the same (Lithium phenyl-2, 4, 6 trimethylbenzoylphosphinate, LAP) and photoinitiator (hydroquinone monomethyl ether, MEHQ). Different sections of sequestration pens on the same microfluidic chip had two different types of hydrogel barriers introduced. The first type was a mid-pen barrier having a thickness of about 15 microns, leaving a culturing region distal to the barrier. The second type of barrier was a hydrogel plug that extended to the distal end of the sequestration pen. F1 formulation barriers were introduced using a 1 to 1.5 second exposure, 10× objective, 50% power for both types of barriers. F2 formulation barriers were introduced using a 3.5 sec to 5 sec exposure, 10× objective, 50% power.

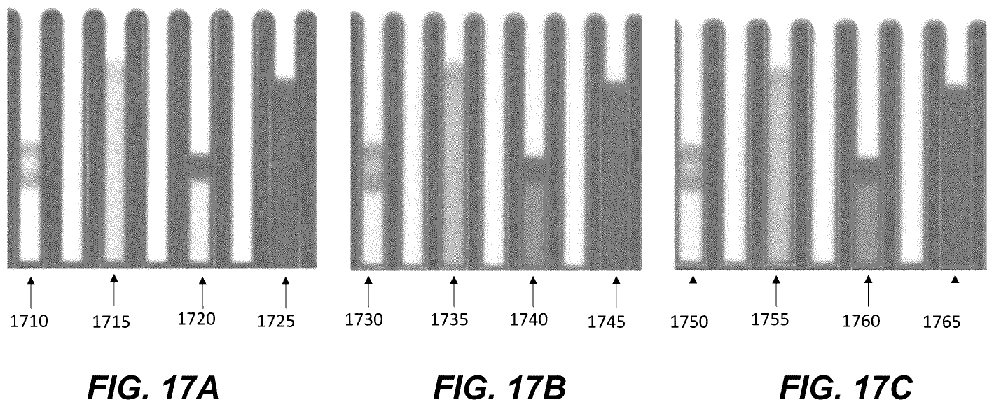

Three different reagent flows were successively introduced. For each reagent flow, images were obtained after a 90 min period of equilibration.
Flow 1: Fluorescent anti-Spot nanobody (MW=30 kDa), shown in FIG. 17A.
Flow 2: Fluorescent anti-SPOT nanobody plus Protein (MW=50-55 kDa), shown in FIG. 17B.
Flow 3: Fluorescent anti-SPOT nanobody plus Protein (MW=50-55 kDa) plus anti-FLAG antibody (MW=150 kDa), shown in FIG. 17C.

As is shown in FIGS. 17A-17C, the low molecular weight labelled nanobody can easily diffuse through the F2 mid-pen 15 micron barrier, as shown in each of pens 1710, 1730, 1750, equilibrating to essentially the same concentration as that of the channel and the area of the pen proximal to the barrier. In FIG. 17A, the hydrogel plug in pen 1715 showed significant amounts of the small labelled reagent, demonstrating that diffusion occurs through the hydrogel and is not due to diffusion around the barrier. Additionally, the barrier having the denser F1 formulation, as shown in pen 1720 also permits diffusion through the barrier, but at a reduced concentration. In pen 1725, the denser F1 formulation hydrogel plus shows quite limited diffusion of the low molecular weight reagent.

In FIG. 17B, where the larger protein is present and which binds to the fluorescently labelled reagent, forming a higher weight complex, affecting diffusion of the bound pair. While not easily seen in pen 1730, the effect is more evident in pen 1735, having the hydrogel plug. The larger bound complex cannot as easily diffuse into the hydrogel, even with the F2 formulation. The denser hydrogels of pens 1740 and 1745 show increasingly decreased amounts of fluorescent reagent, and the large reagent: analyte complex is cannot easily diffuse through.

FIG. 17C shows the effect of adding an anti-FLAG antibody to the reagent mixture, which can also bind to the protein, thus making an even larger tri-component complex. The levels of diffusion were slightly more inhibited in pens 1750, 1755, 1760, 1765.

Further variations: The ratios of 8 arm, 8 acrylamide termini polymer: 8 arm, 1 acrylamide termini polymer may be varied to be in any suitable ratio, e.g., about 10:90; 20:80; 30:70; 40:60: 50:50; 60:40; 70:30; 80:20; 90:10 w/w % or any value therebetween. The molecular weights of the PEG polymers may be varied and do not need to be 20K polymers, but may have a MW of about 5 kDa, 10 kDa, 15 kDa, 20 kDa or more. A consideration in combining polymers of different molecular weights that the rate of diffusion into the pen depends upon the molecular weight. Therefore, the actual ratio of polymers in the pen available for polymerization will therefore reflect the difference in diffusion rates.

### Example 2-2: Hydrogel Geometry for Assaying Soluble Bioproduct

In this example, various hydrogel shapes were formed in the sequestration pen. They were tested for their performance for retaining cells within the distal culturing region of the sequestration pen, as well as assaying productivity, for example, but not limited to using a diffusion gradient assay. Further details of diffusion gradient assays and data analysis thereof is provided below at the section entitled General Diffusion Assay Techniques and in the references described therein.

Pichia cells engineered to secrete a non-endogenous protein were suspended in BMGY (Buffered Glycerol-complex Medium) medium before loading. The engineered non-endogenous protein sequence also included Spot-tag, an inert, unstructured 12 amino acid sequence added to the genetic insert. The additional 12 amino acid sequence is added to a region of the engineered insertion sequence located outside (to the N-terminal end or the C-terminal end) of the desired non-endogenous protein sequence, which can be detected by anti-SPOT nanobodies (Chromotek™). The cells were introduced and disposed into respective sequestration pens by gravity. Hydrogel mixture was introduced into the flow region and allowed to diffuse into the sequestration pens. Then, gel polymerization was initiated by photoactivation at selected area to form various shapes, including full cap (15 um band fully spanning pen, FIG. 7A, barrier 705, center bar (15 microns, as measured in the distal-to-pen opening to proximal-to-pen opening dimension “thickness”×20 microns, “width”, as measured from pen wall to pen wall, forming a pillar centered in the pen leaving a 10 micron gap to the pen walls on each side, FIG. 7A, barrier 710, half bar (15 micron thick band extending from the left wall to the center of the pen, FIG. 7A, barrier 715, and side bars (Two 15 micron thick bands extending from left and right wall leaving an approximately 20 micron gap at the center of the pen, FIG. 7A, barrier 720. The hydrogel barrier(s) were formed in mid-pen and separated the sequestration pen into two areas. The area distal from the opening of the sequestration pen was used as a culture area for cells to live and expand.

Cells were cultured on-chip with constant perfusion of fresh BMGY medium at 30° C. and 80% air fraction (cycle duration was 10 minutes and the flow rate was 0.1 microliters/s) for 17 hours. Then, BMIM medium with 1% MeOH was introduced to induce the secretion for 4 hours (27° C., 5 microliters/s flow rate). After that, BMIM medium with anti-SPOT nanobodies (labeled with ATTO594, Chromotek™) was introduced. The perfusion was continued at 0.014 microliters/s for 45 minutes for equilibration, and then BMIM medium without anti-SPOT nanobodies was introduced and flushed at 0.1 microliters/s for 30 minutes. Images were taken to observe the gradient of soluble fluorescently labeled product at indicated time points.

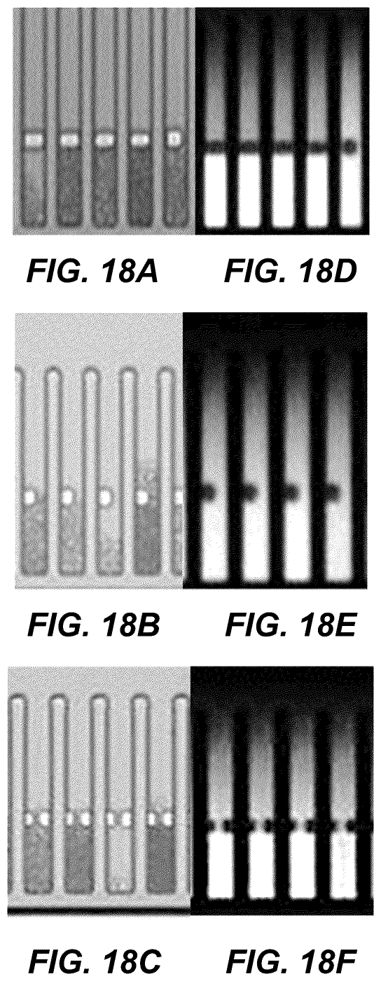

FIGS. 18A-18C shows cell expansion in the sequestration pens and that the hydrogel barrier succeeded in containing the cells within the culturing area, for a variety of hydrogel barrier shapes. In some circumstances, a small number of cells might breach the hydrogel barrier; nevertheless, substantially most, e.g., more than about 80%, more than about 90%, more than about 95% or more than about 99% of all cells were still contained inside the culture area. The results (FIGS. 18D-18F) of a diffusion gradient assay for each respective barrier type, performed as described within Example 2-3, showed that the free anti-SPOT nanobodies rapidly equilibrated in pens with partial hydrogels (center bar, half bar, and side bars) and different intensities can be observed between pens.

### Example 2-3: Bioproductivity Assessment Assays

In this example, four different Pichia strains (Strains 1-4) engineered to secrete a first protein (Protein 1), three different Pichia strains (Strains 5-7) engineered to secrete a second protein (Protein 2), and four further different strains (Strains 8-11) engineered to secrete a third protein (Protein 3), were tested in this assay, but already had known productivities. Each of Proteins 1, 2, and 3 were labeled with Spot-tag, which can be detected by anti-SPOT nanobodies (Chromotek™) as well as the FLAG-tag. Cells were resuspended in PBS before loading and introduced into the flow region. For each stain, single cells were disposed into respective individual sequestration pens using dielectrophoretic forces, which in this example, are optically actuated dielectrophoretic forces. In this example, a single cell was disposed into an individual sequestration pen using positive light actuated dielectrophoretic forces. Details of positive dielectrophoretic transport of cells are described in International Application Serial No. PCT/US2020/066229, entitled “Methods of Penning Micro-Objects Using Positive Dielectrophoresis”, filed on Dec. 18, 2020, and published as International Patent Application Publication WO2021/127576, the entire disclosure of which is herein incorporated by reference in its entirety for any purpose. However, the invention is not so limited, for example, when other cell types such as bacterial cells, negative dielectrophoretic forces may be used to selectively place a single cell (or more than one cell) into each individual sequestration pen.

Hydrogel formation. In assays where hydrogel barriers were used in the productivity assay, flowable hydrogel polymer was introduced in solution, and allowed to diffuse into the sequestration pens. Photoinitiator was also included within the solution containing the flowable hydrogel polymer. The hydrogel barriers were formed by photopatterning, e.g., photoactivation of polymerization to form the solidified hydrogel barriers. The formed hydrogel defined the sequestration pen into two regions (e.g., areas). The region that was furthest away from the opening and contained cells therewithin was a culture area. In some instances, the other region that was closest to the opening was an assay area (See FIG. 8A), for example using a bead assay and a hydrogel barrier.

Variation 1. Culturing, Induction and Assay using Capture Bead. Strains 1-11 were used in this experiment. The hydrogel formulation included 8 arm 20K PEG having 8 acrylamide termini, included inhibitor (Lithium phenyl-2, 4, 6 trimethylbenzoylphosphinate, LAP) and photoinitiator (hydroquinone monomethyl ether, MEHQ). A nitrogen gas purge followed the introduction of the flowable polymer mixture. Gel polymerization was initiated by photoactivation in the DAPI filter cube, using 10× objective, for Is at 50% power. Gel polymerization was initiated by photoactivation at mid-pen to form a cap formed from two individual triangular gel barriers, like the barrier shown in FIG. 7A, barrier 725. The nominal size of the illumination used to form the barrier produced a “bowtie” shape with about a gap in the center) in each pen. Depending on the exposure time, exact amount of initiator, inhibitor and/or polymer composition, the triangular gel barriers may meet in the center rather than leaving a discernable gap. In other instances, the triangular barriers may leave a gap that is less than about 1, 2, 3, 4, 5, 6, 7, 8, 9 or about 10 microns wide. “Bowtie” barriers can function equally well in either case. What these barriers share is a non-uniform thickness. That non-uniformity may be in the width (pen wall to pen wall dimension), in the “thickness” dimension (from distal-to-pen opening to proximal-to-pen opening dimension), and/or the “height” dimension (from inner surface of the substrate to the inner surface of the cover of the microfluidic device, e.g., z-dimension). The non-uniformity in the “thickness” dimension, e.g., the center of the “bowtie”, provides a convenient and standard location for bead disposition for the assay. The “bowtie” barrier had a “thickness” at its largest dimension of the triangular shaped individual gel form of about 15 microns. This dimension is not limiting and the “thickness” of the bowtie may be less than about 15 microns, less than about 12 microns, less than about 10 microns, less than about 8 microns, or less than about 5 microns. In other embodiments, the “thickness” of a triangular segment of the “bowtie” barrier may be more than about 10 microns, more than about 15 microns, or more than about 18 microns. As shown in FIGS. 7B-7C, two different embodiments of hydrogel barriers having a “bowtie” non-uniform shape are shown, where the dimension 760 of the triangular segment forming the barrier (“thickness” dimension) is larger in the barrier of FIG. 7C than the dimension of the segment 750 of the barrier of FIG. 7B. Since the pens are of the same width, it can be seen that the barrier formed in FIG. 7B has a distinct gap between the two barriers, while the barrier of FIG. 7C does not have an obvious gap. While in the embodiment of FIG. 7B, a few cells have escaped the culture area below the barrier, the barrier of FIG. 7C does not permit cells to move past the barrier.

Further variations. While the “bowtie” non-uniform barrier was used in this experiment, other non-uniform barriers may be suitable barriers for productivity assays using beads to capture secreted biomolecules, and may include some of the non-uniform barriers shown in FIG. 7A. Depending on culture period duration and the specific type of cells being assayed, many different configurations of barriers will work. The specific type of cell may determine what type of barrier may be used, as cells that tend to grow in close association within the culturing region of the divided pen, may not even require a barrier that blocks more than half the width of the pen.

Cell culture, Bead Load, and Induction. Cells were then cultured on chip with constant perfusion of fresh BMGY medium at 30° C. and 80% air fraction (cycle duration was 10 minutes and the flow rate was 0.1 μL/s) for 14 hours. The period of time for culturing may be selected as desired, and may be less than about 14 h, 12 h, 10 h, 8 h, or less or may be more than about 10 h, 12 h, 14 h, 16 h, 18 h, or about 20 h. Overgrowth of the culturing area may be a determining factor in selecting the culturing period.

Assay beads coated with anti-FLAG antibodies which bind to the FLAG® peptide sequence) were suspended in loading buffer and then introduced into the flow region before the induction. Single beads were disposed into each pen and located in the assay region. In some further variations, a second period of culturing may be added after bead introduction. This period of culture may be for about 1 h, 2 h, 3 h, 4 h, or any value therebetween, and assures that the cells are in a state such that induction will be more uniformly successful. Flow of liquid media (BMGY) is alternated with air perfusion, in a ratio of 20% liquid: 80% gaseous (air) in a 10 min cycle, at 1 microliter/sec.

After introduction of the bead (and in some variations, after the second period of culturing), BMIM medium with 1% MeOH was introduced to induce secretion of the molecule of interest (the analyte). The induction was performed for 5 hours with the perfusion of the BMIM medium continued (1 microliter/s, 27° C.). In some variations, the induction period is varied from a 5 h period, and may be selected to be about 1 h, 2 h, 3 h, 4 h, 6 h, 7 h or more.

Assaying. BMIM medium with anti-SPOT nanobodies (labeled with ATTO594, Chromotek™) was introduced. The perfusion was continued at 0.011 μL/s for 30 minutes for equilibration, and then BMIM medium without anti-SPOT nanobodies was introduced and flushed at 5 μL/s for 60 minutes. Brightfield images were taken before assaying for biomass measurement, which was used to normalize the results of the measurement.

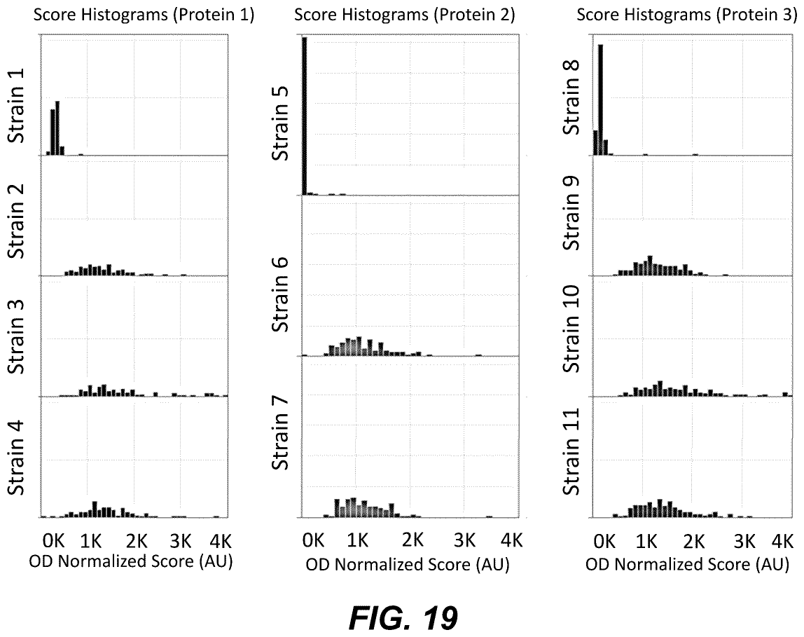

Mean bead fluorescent signal was detected and normalized with biomass (OD score). The results were shown in the histograms of FIG. 19 showing the production results for Protein 1, Protein 2, and Protein 3 (from left column to right column) for respective Strains 1-11. The x-axis shows the OD normalized score (AU) (marked at 1K, 2K, 3K, and the y-axis shows count (0 to 100). The histograms match the expectation of productivities as Strain 1, Strain 5, and Strain 8 were expected to be the worst producers of the respective proteins of the tested groups. Furthermore, it was observed that there were different levels of productivities between sequestration pens containing cells from the same strain. The locations of the highest scoring pens were stored by the analysis software of the Beacon system and can be used to export the cells exhibiting high productivities in this assay. Therefore, the better producers from each strain may be exported for expansion and further development.

Further variation. In another variation, the “bowtie” or other non-uniform hydrogel barrier may be introduced, where the hydrogel is permeable to the secreted analyte or where there is a gap between portions of the hydrogel barrier. The secreted analyte, being a soluble product may diffuse out of the culturing area, through the hydrogel and/or through a gap in portions of the hydrogel barrier. A diffusion gradient assay may then be performed in an area of interest that is selected to be located between the opening of the pen into the channel and the surface of the hydrogel facing the opening of the pen into the channel (“assay area” as shown in FIG. 8B). BMIM medium with anti-SPOT nanobodies (labeled with ATTO594, Chromotek™) is introduced, and the soluble analyte is labelled with the anti-SPOT nanobodies. Many variations of diffusion gradient assay imaging may be used, and details of these variations are provided below at the section entitled General Diffusion Assay Techniques and in the references described therein.

Variation 2. Accumulation Assay. In another variation, secreted product was accumulated within the same region as the cells, below a hydrogel barrier which substantially spans the width of the sequestration pen, as described herein (e.g., located within a pen to divide the pen into two regions, distal and proximal to the opening, forming a full cap as shown in FIG. 7A, barrier 705. Cells were cultured within the distal, culturing region. An area of interest for monitoring the amount of detectable product was located within the culturing region, but in a portion of the region that preferably does not contain any cells. For example, a portion of the culturing region closest to the distal side of the hydrogel barrier was defined as the area of interest for the assay. The area of interest typically may not include any portion of the gel itself, when performing this assay. An accumulation-type assay may be useful where the culturing cells produce the bioproduct at a low rate, which may be difficult to detect using a bead to capture secreted bioproduct.

Cell culture and Induction. After introduction of individual cells into respective sequestration pens, as described above for Strains 1-11, cells were then cultured on chip with constant perfusion of fresh BMGY medium at 30° C. and 80% air fraction (cycle duration was 10 minutes and the flow rate was 0.1 μL/s) for 14 hours. The period of time for culturing may be selected as desired, and may be less than about 14 h, 12 h, 10 h, 8 h, or less or may be more than about 10 h, 12 h, 14 h, 16 h, 18 h, or about 20 h. Overgrowth of the culturing area may be a determining factor in selecting the culturing period.

BMIM medium with 1% MeOH was introduced to induce secretion of the molecule of interest (the analyte). The induction was performed for 5 hours with the perfusion of the BMIM medium continued (1 microliter/s, 27° C.). In some variations, the induction period is varied from a 5 h period, and may be selected to be about 1 h, 2 h, 3 h, 4 h, 6 h, 7 h or more.

Accumulation Assay. BMIM medium with anti-SPOT nanobodies (labeled with ATTO594, Chromotek™) was introduced. The perfusion was continued at 0.011 microliters/s for 30 minutes for equilibration, and then BMIM medium without anti-SPOT nanobodies was introduced and flushed at 5 microliters/s for 60 minutes. Brightfield images were taken before assaying for biomass measurement, which was used to normalize the results of the measurement. Fluorescent Images were taken during flushing to determine signal intensities of the anti-SPOT nanobodies within an area of interest that was selected to be within the culture area of each pen.

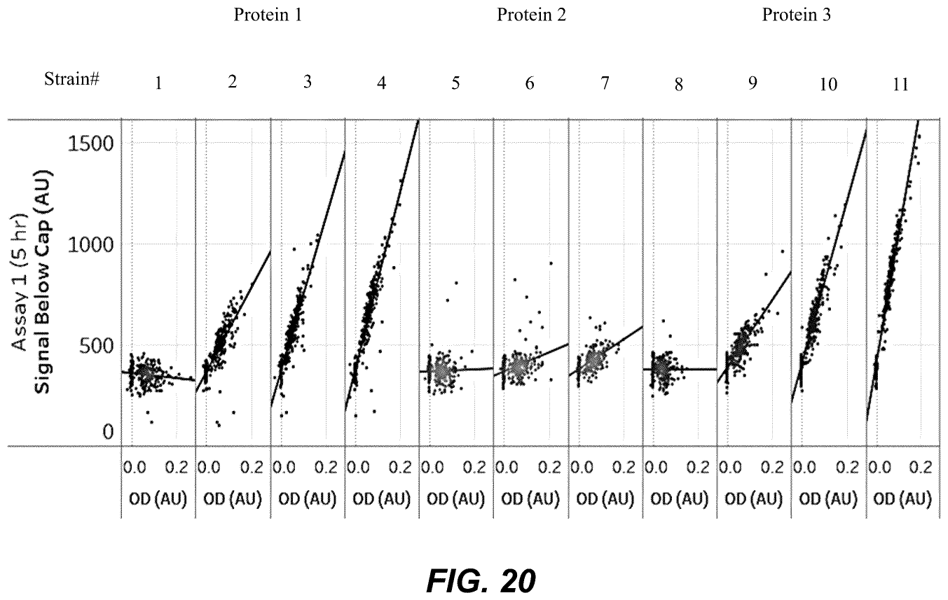

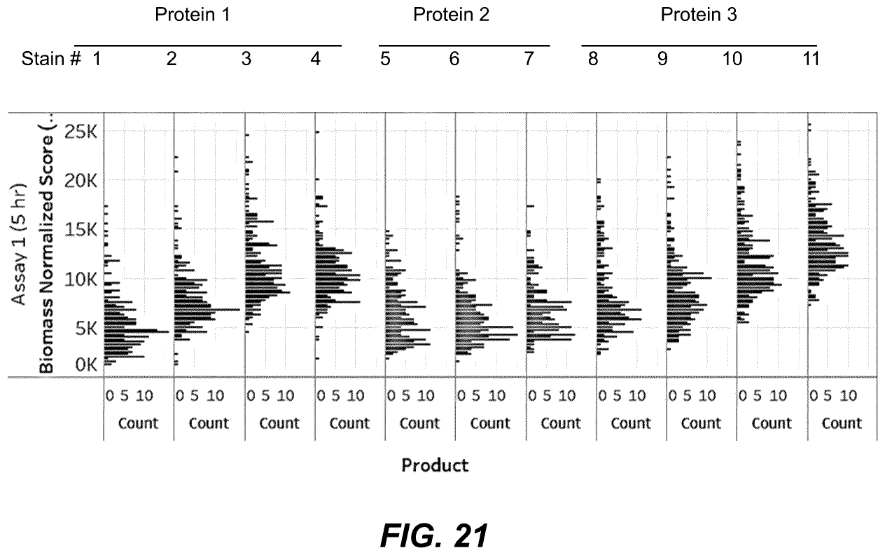

The results, shown in FIG. 20, showed that the presence of a hydrogel fully sealing the pen results in concentration of secreted analytes within the culture area that can then be detected by using the anti-SPOT nanobodies. The x-axis shows biomass, as measured by OD, and the y-axis shows signal intensity in the area of interest below the hydrogel barrier. A linear correlation with colony size demonstrates that this method of quantifying secretion is highly predictive of productivity. FIG. 21 shows histograms of the signals normalized to the OD, where the y-axis shows the score (e.g., detectable signal intensity) normalized for biomass and the x-axis shows the number of pens having each score. The results were consistent with expectation, and the distribution of scores provides additional information for selecting a better producer. For example, Strain 3 and Strain 4 were overall better producers than Strain 2. Nevertheless, some pens of Strain 2 exhibited signals stronger than most of the pens of Strain 3 and Strain 4. Accordingly, the diversity of secretion productivity within a given strain can be captured and selection of individual clonal populations from a strain can yield superior candidates for further development.

### Example 2-4: Bioproductivity Assessment Using Accumulation Assay

In this example, three Pichia strains, Strain 2, Strain 3, Strain 4, and Strain 11 (the same as in Example 2-3) were assayed for their productivities. The Protein 1 was labeled with Spot-tag, which can be detected by anti-SPOT nanobodies (Chromotek™). Cells were resuspended in PBS before loading and introduced into the flow region. For each stain, single cells were disposed into the respective sequestration pens by OEP, as described above in Example 2-3. Fluorescently labeled dextran TRED was used for in-pen correction.

Hydrogel formation. Then, PBS including a mixture of 75%8 arm 20K PEG having 8 acrylamide termini) and 25%8 arm PEG having only one acrylamide terminated arm hydrogel mixture was introduced and allowed to diffuse into the sequestration pens. The flowable polymer mixture also included photoinitiator (Lithium phenyl-2, 4, 6 trimethylbenzoylphosphinate, LAP) and inhibitor (hydroquinone monomethyl ether, MEHQ). A nitrogen gas purge followed the introduction of the flowable polymer mixture. Gel polymerization was initiated by photoactivation in the DAPI filter cube, using 10×, for 3 s at 50% power, at mid-pen to a fully sealing cap for the accumulation assays. The formed hydrogel defined a culture area within the sequestration (See FIG. 8C, and FIG. 7A, barrier 705).

Cell culture and Induction. Cells were then cultured on chip with constant perfusion of fresh BMGY medium at 30° C. and 80% air fraction (cycle duration was 10 minutes and the flow rate was 0.1 microliters/s) for 14 hours. After culture, BMIM medium with 1% MeOH was introduced to induce the secretion of the molecule of interest (the analyte). The induction was performed for 5 hours with the perfusion of the BMIM medium continued (5 microliters/s, 27° C.).

Assaying. BMIM medium with anti-SPOT nanobodies (labeled with ATTO594, Chromotek™) and BMIM medium with 10 kDa Dextran (Texas Red™, #D1828, Thermo Fisher) were introduced. The perfusion was continued at 0.007 microliters/s for 85 minutes for equilibration. Fluorescent Images were taken to determine signal intensities of the anti-SPOT nanobodies and dextran within the culture area of each pen for measurement under both equilibrium conditions and flush conditions. Further details of diffusion gradient assays and data analysis thereof is provided below at the section entitled General Diffusion Assay Techniques and in the references described therein. Brightfield images were taken for biomass measurement before assay, which were used to normalize the results of the measurement for biomass (OD).

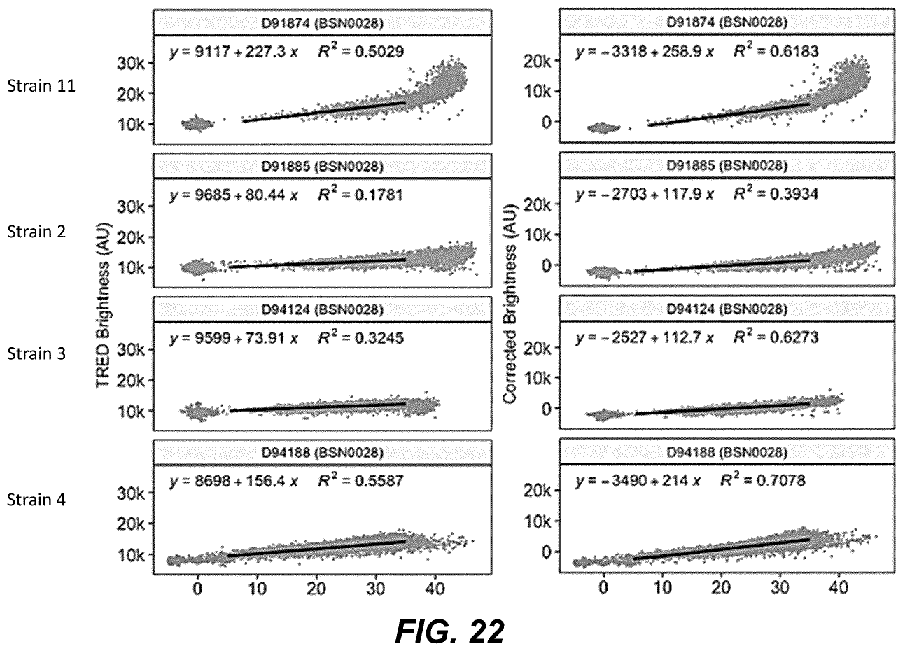

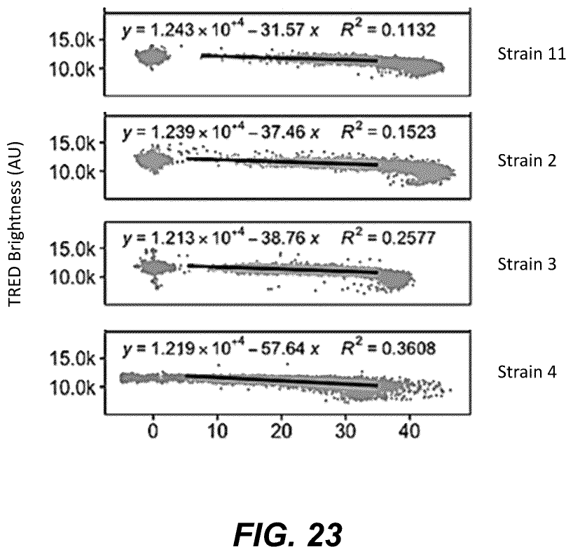

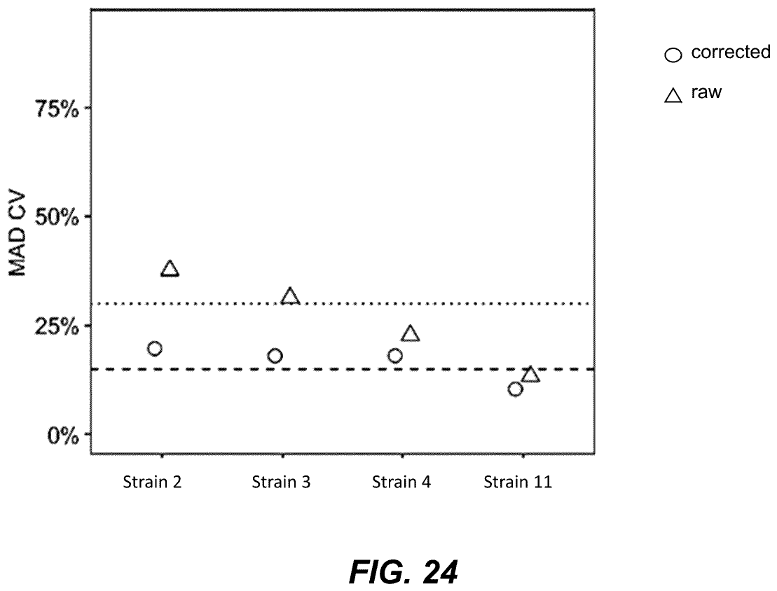

The graphs shown in the left column of FIG. 22 shows the detected signal (raw) of anti-SPOT nanobodies again OD score for each of Stains 2, 3, 4, and 11. Strain 11 and Strain 4 exhibited better secretion than Strain 2 and Strain 3, which were consistent with the known productivities of these strains. The graphs in the right hand column of FIG. 22 shows the same data as that of the graphs in the left hand columns, but corrected against the fluorescent dextran values across the microfluidic chip. FIG. 23 shows the detected signal of in-pen dextran used for the corrections. Because dextran was not secreted by the cells and also did not bind the cells, the signals observed for TRED labelled dextran were flat against the OD score and used as an in-pen control correcting the signals of anti-SPOT nanobodies. The corrected signals of anti-SPOT nanobodies were shown in the right column of FIG. 22. The correction significantly reduced the coefficient of variation (Median absolute deviation coefficient of variation; MAD CV) as shown in FIG. 24.

Example 2-5. Export. Selected pens are prepared in order to export the cells within the culture area, and a sequence of photographs illustrating the export process is shown in FIGS. 25A-25F. In FIG. 25A, the non-uniform hydrogel barrier used for the bead assay (“bowtie”) is annotated for clarity as seen in the figures, for example barrier 2505, as well as the uniform barrier 2510. The central pen was the only pen in this view for which export was desired. At this first time point, for all pens for which export is not desired, additional uniform hydrogel barriers 2510 were already introduced, which prevents cells from exiting those pens. This was performed in case any cells in non-desired pens were located in the area proximal to the opening of the pen, thus reducing risk of clonality loss for the cells being exported. Therefore, the non-selected pens have two hydrogel barriers, and the selected pens have a single barrier. Starting export of cells 2515, a light actuated dielectrophoretic bar was programmed to move towards the opening of the pen to the channel. The dielectric force in this instance repels the cells ahead of the light 2520, which is the visible light activating transistors in the substrate, inducing the negative dielectrophoretic force. At a second, later timepoint, as shown in FIG. 25B, the light bar 2520 is shown at a location closer to the opening of the pens, having activated the transistors within the substrate, thus moving the cells closer to the hydrogel barrier 2505. The non-uniform hydrogel barrier, however, prevented cells from moving past the barrier.

At a third still later timepoint, as shown in FIG. 25C, a laser pulse had been directed towards an area of the substrate most distal to the opening of the pen, and the location of the effect 2530 is shown. The dielectrophoretic force had already cleared cells from that portion of the culturing area, so direct impact of laser illumination on cells was prevented. The z-focus was changed for this individual photo, and the focus was resultingly affected. However, it is clear that the laser pulse induced heating and/or a bubble in the media that deformed two triangular segments forming the non-uniform “bowtie” barrier. The two segments 2525 and 2525′ have flipped within the pen from their original position, and now provided a passage for cells to move through. The heating (including expansion of the media) or bubble exerts a force towards the proximal end of the pen, e.g., towards the opening of the pen, and therefore exerts a force upon the center of the hydrogel barrier. The “bowtie” non-uniform hydrogel barrier permitted deformation in the center, and more easily deformed to permit passage of cells.

At a fourth time point, as shown in FIG. 25D, (z focus is resumed once the laser is not operating) where the sweep of the pen with the light bar 2520 was resumed, moving cells past the deformed barrier segments 2525 and 2525′ (also annotated for clarity) that no longer prevent cell transport. Another light bar 2522 preceded the central group of cells, the dielectrophoretic forces inducing clearance of the path for the cells. At a fifth subsequent time point, as shown in FIG. 25E, the light bar 2520 continues moving the cells towards the opening of the pen, which opens into the channel at the top of the figure. Finally, at a sixth timepoint, as shown in FIG. 25F, the light bar 2520 has initiated dielectrophoretic forces that transported the cells 2515 into the channel. A third light bar 2524 swept through the pen, following the first group of cells 2515, to initiate dielectrophoretic forces to capture any remaining cells into the channel to be joined with the first group of cells. After this timepoint, flow was started in the channel to carry the selected cells to an external vessel such as a well in a wellplate. The cells may be further expanded or processed in another manner.

### Experiment 3: Yeast Productivity

Cell Materials: All strains used in this study were derived from CEN.PK113-7D, a model laboratory strain of Saccharomyces cerevisiae

Single-Cell Loading. Broth from microplate precultures or mutagenesis recovery was diluted in PBS to a target OD₆₀₀ of 0.1 and transferred to a 300-uL 96-well plate for import (Corning). The import plate was then loaded into the well-plate incubator on the Beacon instrument for single cell loading. During the loading process, the temperatures of the well-plate incubator and the OptoSelect chips were set at 4° C. and 18° C., respectively, to reduce cell proliferation during the loading period.

For each strain to be loaded into the NanoPen chambers, 25 μL of cell suspension was imported from the well-plate incubator to the channels of the OptoSelect chips. Single cells were identified automatically by the Beacon instrument control software using a convolutional neural network algorithm optimized for yeast cells. A positioning strategy was then automatically implemented to maximize loading throughput using the OEP technology. Residual cells in the channels were flushed to waste after loading. In general, it took roughly 4-5 hours to load the cells across all 4 chips in a single library screen workflow.

Culture and biomass Measurement. After cell loading, the cells were cultured on-chip at 33.5° C. with constant perfusion of fresh media (BSM+carbon source) at 0.1 μL/s. Time-lapse bright-field images were taken every hour to keep track of cell growth for each colony. Depending on the experiment, one of two types of productivity measurements was performed within the culture period: the sealed-pen assay or the open-pen assay, as described below.

Example 3-1: Sealed-chamber productivity and yield inference. Although there are many ways to transduce a fluorescent signal in response to the local concentration of an analyte, detection is easiest when the product is intrinsically fluorescent. A set of S. cerevisiae strains engineered to produce a fluorescent small molecule was thus chosen to demonstrate the feasibility of screening for a secretion phenotype.

While work has been performed on secretion of macromolecules, with sufficiently slow diffusion to allow accumulation and visualization using fluorescent readouts for low-molecular-weight compounds (<1 kDa) as investigated here, rapid diffusion from the chambers was initially a concern. A method was therefore devised to seal the NanoPen chambers using a hydrophobic oil with high respiratory gas solubility, but low product and carbohydrate solubility. After sealing, each chamber effectively becomes a miniature batch reactor with fixed carbon source, accumulating product, and continuous gas exchange. When the assay is complete, the oil can again be replaced with aqueous media to allow export of desired strains.

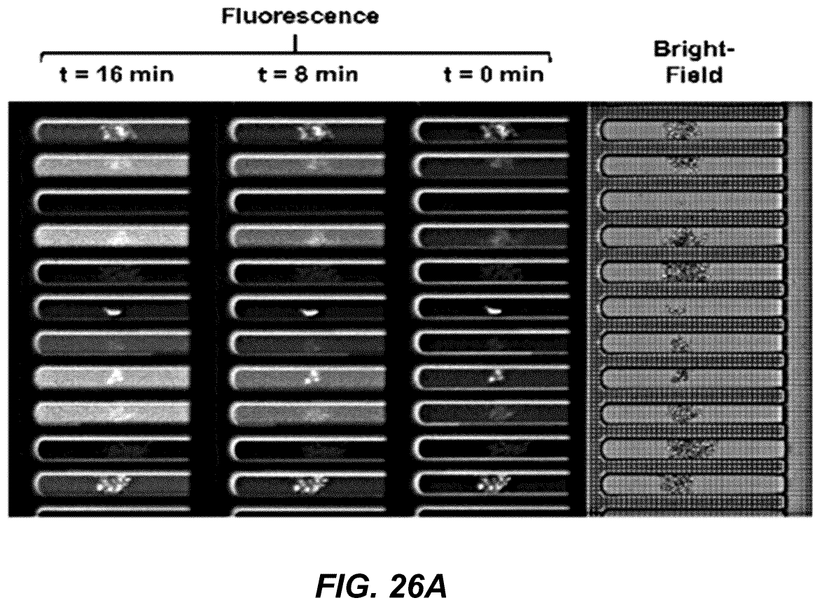

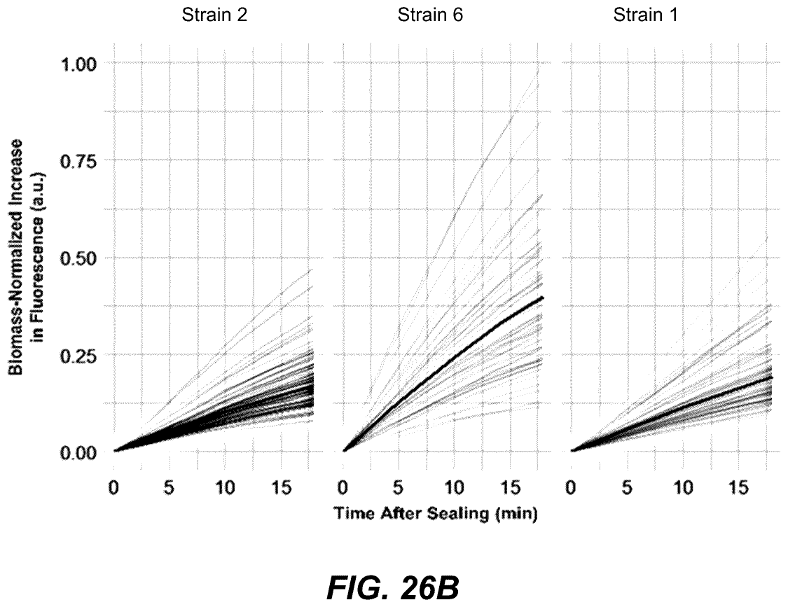

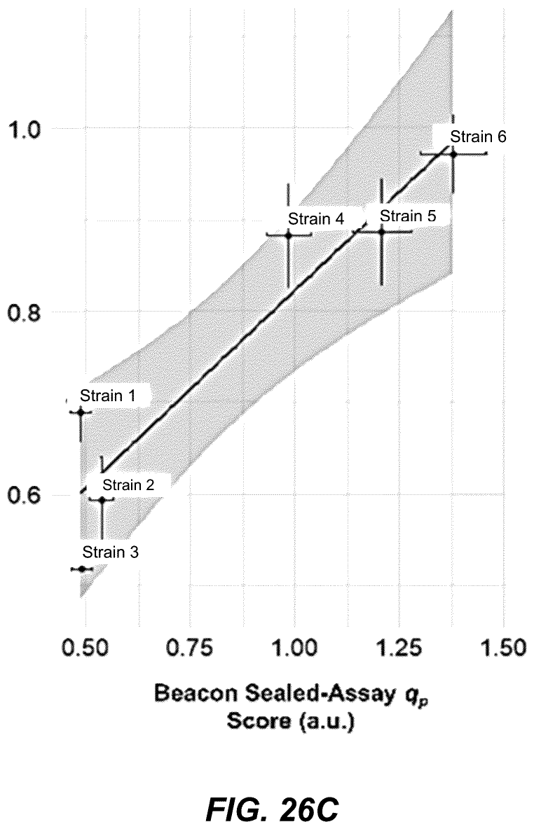

To determine whether strain performance in the sealed-chamber assay aligns with lab-scale bioreactor performance, six strains previously tested in 0.5-L bioreactors were selected. The strains were sequentially disposed into NanoPen chambers on OptoSelect chips within the microfluidic controlling system in high replication (n=40-50). Strains were cultured for ˜18 hours. Fluorinated oil (HFE-7500) was imported into the main channel to seal each chamber and thereby mitigate diffusion of the secreted product into the main channel. The increase in fluorescence of the secreted product was tracked during incubation for an additional 20 minutes using periodic fluorescence imaging (FIG. 26A), allowing inference of the relative P for each NanoPen from the slope of the intensity curve for each colony during the 20-minute interval. To compare the per-cell metabolic fluxes generated by each strain, relative specific productivities for the cells in each chamber were calculated by normalizing the slope of the fluorescence intensity curve to the measured OD score, giving what is hereafter referred to as the “qₚ score” FIG. 26B shows data for Strain 2. Strain 6, Strain 1, where the x axis is time after sealing in minutes, and the y-axis is biomass-normalized increase in fluorescence. Note that the precise definition of qₚ includes generation of both secreted and intracellular product; however, the strains tested in this work predominantly secrete (e.g., the vast majority of) the fluorescent product. Therefore, rough relative inferences of qₚ can be made from the biomass-normalized measurement of secreted product in this context.

The microfluidic qₚ scores clearly distinguished three top performing strains from three weaker producers (FIG. 26C). Mean qₚ scores from the microfluidic assay showed positive correlation (R²>0.85) with the qₚ measured in bioreactor experiments in the 24-48-hour interval. Therefore, the microfluidic system and assays provides a relevant culture model for the selection of the most promising strains for bioreactor testing.

Example 3-2. Long-Term Sealed Chamber Assay. In addition to the 20-minute productivity measurement, the sealed-chamber method was used to assess the average yield of product with respect to feedstock. After cell loading, the chip was perfused with production media containing 3% sucrose for 60 minutes to supply fresh media to all chambers. Fluorinated oil (HFE-7500) was then imported into the main channel to seal each pen for 24 hours. The increases in fluorescence and biomass were monitored over the 24-hour duration by acquiring images every 30 minutes. To supply oxygen to the oil-sealed chambers, 5 microliters of oil was pushed back and forth through the chip at 0.2 μL/s every 20 minutes. The tubing leading to the chip was highly gas permeable, which allowed reoxygenation of the fluorinated oil. The oxygen solubility of the oil is 100 mL gas/L oil, which is 25-fold higher than that of water (4.8 mL gas/L water). Therefore, the oil perfusion is considerably more effective in oxygenating the chambers than media at similar flow conditions.

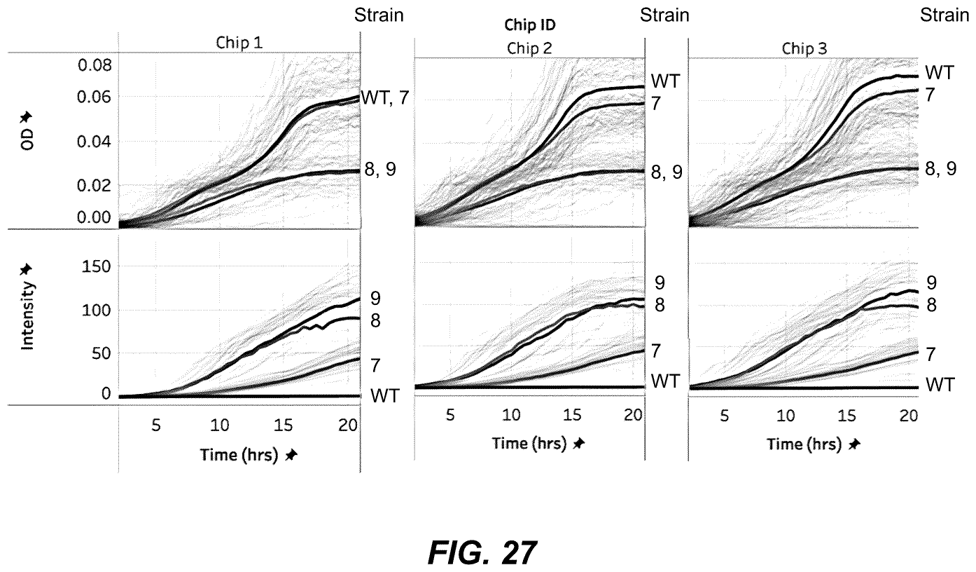

FIG. 27 shows the increase of biomass and product concentration in each pen during the 24-hour sealed-chamber yield assay. In total, 4 strains were assayed: one wild type (Strain WT), one low producer (Strain 7, ˜90 mg/L product titer in microplates), and two moderate producers (Strain 8 and Strain 9, each with ˜450 mg/L titer). Three replicates are shown in FIG. 27. Consistent with behavior in plate culture models, the wild type and weak producer accumulated more biomass, while the moderate producers generated a higher fluorescence intensity, indicating that resources were shunted toward product pathway flux. The 24-hour sealed-chamber cultures of four strains gave endpoint fluorescence values that ranked them consistently with the relative titers attained in microplate models after carbon sources had been exhausted, demonstrating comparability with macroscale cultures.

Tracking growth and productivity under the constraint of limited feedstock may provide a direct comparison of resource utilization from strain to strain, a readout complementary to the real-time productivity measurements from the open-chamber assay. Because many commercial fermentations are run in fed-batch rather than continuous perfusion conditions, high product and byproduct accumulation can present an obstacle to high performance. Using oil to block the efflux of product may help to apply selective pressure in screening efforts to reduce feedback inhibition and increase product tolerance; likewise, blocking the efflux of toxic byproducts may help screen for strains that produce them at lower concentrations. Therefore, this sealed-chamber format can be a complementary tool for assessing strain resilience to produce and by-product accumulation.

Example 3-3: Real-time productivity monitoring via steady-state product gradient assay (Open-chamber assay). The rapid development of fluorescence signal in the sealed-chamber assay prompted investigation of an alternative method, in which pens are left unsealed throughout the experiment. In this assay, local product concentration within each chamber is mainly governed by the rate of production by the colony and diffusion away from the colony. As a result of the effective boundary condition just outside of each chamber (product concentration≈0), the concentration gradient in the cell-free gradient measurement area at steady state will be proportional to the rate of secretion of the analyte. This allows for inference of the productivity in each pen in the few seconds preceding a single fluorescence image of the chip by simply extracting the slope of the linear fit of signal intensity with respect to position in the chamber. Further details of diffusion gradient assays and data analysis thereof is provided below at the section entitled General Diffusion Assay Techniques and in the references described therein. As images were acquired periodically, time-dependent productivity data for every colony was collected throughout the experiment. Strain productivity was high enough to enable quantitation of productivity within hours of initiating incubation.

Open-pen real-time productivity assay. The open-pen assay measured the steady-state position-dependent fluorescence gradient in each NanoPen during media perfusion. In this gradient, the cells were the source and the channels the sink for the fluorescent product, maintaining a gradient proportional to the secretion rate of the cells. During the open-pen assay, the media perfusion rate was raised to 0.3 μL/s for 10 minutes to establish steady chemical gradients from the NanoPens to the channels before taking the fluorescence images. The only requirements for this assay were to set a sufficiently high flow rate to maintain a uniform sink in the channel and sufficient time to reach a steady state. The mechanics of this assay were such that measurements could be captured at a 12-minute interval on 1 chip without significant interruption to culture. For routine experiments, the open-pen assay was performed at 1-hour intervals to measure the productivity of each colony across 4 chips running in parallel.

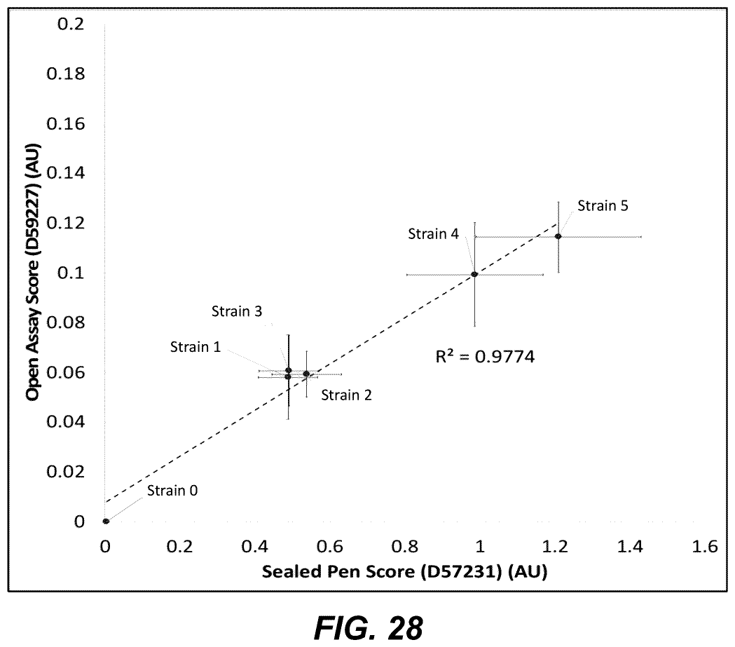

To check the comparability of the sealed- and open-chamber assays, six strains including five of the six strains tested in the sealed-chamber assay discussed above were subjected to the new format using identical culture media. The mean qₚ scores for each strain from the two assays correlated extremely well (R²>0.98, FIG. 28) and the variability was comparable, with most strains showing CVs around 15-20%. The open assay was chosen as the primary assay method for correlation analysis and library screening because of its additional advantages, including temporal resolution and minimal interruption during culture.

Example 3-4. Identification of optimized culturing conditions in the open chamber assay. An expanded set of 11 strains that had exhibited varying performance in 0.5-L bioreactors was selected to help identify optimal chip culture conditions for screening (Table 1). Using the real-time data made available by the gradient productivity assay, several media compositions and assay durations were evaluated to maximize correlation of qₚ score to values of Y, P, and qₚ measured from bioreactor fermentations. In each experiment, an R² value was computed for the linear regression of the bioreactor metric with respect to the mean chip score at a given time. A plot was then constructed to show the evolution of the correlation strength over time to aid in the selection of assay duration. Ultimately, the conditions that optimized prediction of bioreactor performance resulted in a strong positive correlation (R²=0.82) between mean qₚ score and 24-48 hour interval bioreactor qₚ for a set of 11 diverse engineered strains. As a comparison, endpoint titers in microplate culture model tests of the same strains yielded at best a correlation of R²=0.73, also with respect to bioreactor qₚ. The low sugar culture conditions generally have worse chip-to-bioreactor correlation, lower stability in productivity over time, lower dynamic range, and larger assay CV. The 1.5% glucose+1.5% fructose was therefore selected to be the carbon source for on-chip production.

TABLE 1
Head-to-head comparison between various media compositions used in the open-
chamber assay. 1.5% glucose + 1.5% fructose was selected as the carbon source
in the PR media. The maximum chip-to-bioreactor correlation was selected by
tracking the Pearson's R observed between microfluidic qₚ score and bioreactor
peak qₚ, Y, and P as a function of microfluidic assay time.

| | 3% | 3% | 1.5% Glucose + | 0.01% | 0.01% | 0.005% Glucose + |
|---|---|---|---|---|---|---|
| | Sucrose | Glucose | 1.5% Fructose | Sucrose | Glucose | 0.005% Fructose |
| Maximum chip-to- | 0.80-0.84 | 0.71-0.77 | 0.77-0.82 | 0.23-0.32 | 0.67-0.70 | 0.64-0.67 |
| bioreactor correlation | | | | | | |
| (observed Pearson's R) | | | | | | |
| Consistency in chip-to- | High | High | High | Low | Low | Low |
| bioreactor correlation | | | | | | |
| over culture period | | | | | | |
| Dynamic range of | High | High | High | Low | Medium | Medium |
| observed qₚ scores | | | | | | |
| Typical variability in | Low | Low | Low | Medium | Medium | Medium |
| qₚ score for each strain | | | | | | |
| Assay period | 10-24 h | 10-24 h | 10-24 h | 40-50 h | 10-20 | 10-20 |

Example 3-5: Two Tier Screening. Productivity-based screening identified hits exhibiting increased peak qₚ in lab-scale bioreactors. Four strain libraries were generated by random whole-genome chemical mutagenesis of strains containing proprietary engineering for overproduction of the fluorescent target. As a point of reference, these parent strains had previously been shown to produce the target at titers reaching 15-22 g/L in lab-scale bioreactors. Libraries were screened using a two-tiered scheme of the present disclosure.

In Tier 1, four chips were dedicated to screening each clone at n=1, with a total throughput of ˜4,000 clones per library. After selection and export of 44 high-scoring clones (hits), strains were cultured to accumulate biomass in a 96-well plate. Hits were then submitted to Tier 2 screening on two additional chips, in which each strain was cultured in higher replication (typically n˜50-100) to identify strains for promotion to bioreactors with higher confidence

Each Tier-2 screen identified at least one mutant with mean qₚ score improved over the parent strain by >20%. Seven mutants improved by varying extents were then tested alongside their parent strains in ambr250 bioreactors to assess performance at lab scale. Four of the seven strains exhibited peak bioreactor qₚ values improved over parent by 10-85%. The strain with the largest bioreactor qₚ improvement additionally achieved a 20% increase in average Productivity over a 6-day fermentation, making it a lead candidate for further engineering.

These results indicate that the open-pen assay can deliver throughput and reproducibility comparable to a microplate screen of 2000-6000 mutants, in a variety of background genotypes.

Example 3-6: Unload Colony export. The colony export process of cells in this experiment included a series of operations prior to OEP unloading-colony unpacking, pruning, and extended PBS flush, in order to reduce cross-pen contamination from the overgrowing colonies. Throughout the export process, chip temperature was set at 18° C. to reduce metabolic activity of the cells. DI water (500 microliters) was first perfused across the chips for 10 min. The osmotic pressure led to the swelling of cells, unpacking the tightly aggregated colonies. Immediately afterwards, an OEP sequence was performed to prune away excessive cells from the chambers. The Beacon system software utilized a convolutional neural network-based cell detection algorithm to keep track of pens with potential clonality risk. PBS was perfused through the chips continuously at 0.5 μL/s during the pruning process to flush out excessive cells into the waste bottle. After the pruning process, an extended PBS flushing operation was performed at 1 microliter/s for an hour to minimize potential sources of contamination from cells trapped along the fluidic path.

After the series of clonality de-risking operations, the OEP unload sequence was performed to export the chosen best clones identified in the assay. Each colony of interest was unpenned by OEP into the channels and delivered into the 96-well export plate (Corning), where 150 μL of rich growth media was loaded in each well. Blank exports and colony exports were performed alternately to assess clonality control. The 96-well export plate was kept at 4° C. in the Well-Plate Incubator during the export process and switched to room temperature at the end of the workflow. The entire colony export process took about 15 hours for 4 chips with 48 colonies retrieved in total, but can be easily optimized by a wide margin for higher efficiency. The export colonies were then transferred to a 1.6-mL round-well 96-well plate (Axygen) for outgrowth.

### Additional Materials and Methods

Media and Reagents. Chemicals were purchased from Thermo Fisher Scientific unless indicated otherwise. All media components were filter sterilized, with the exception of yeast extract, Bacto Peptone, and agar, which were autoclaved before addition of other components.

Solid media contained yeast extract (10 g/L), Bacto Peptone (20 g/L), agar (20 g/L), maltose (30 g/L), and lysine (2 g/L). A modified solid medium containing glucose (20 g/L) and maltose (10 g/L) instead of 30 g/L maltose was used to prepare biomass for ambr250 bioreactor experiments.

Rich growth media was used for biomass accumulation prior to some experiments and for recovery from whole-genome random mutagenesis. The rich growth media contained yeast extract (10 g/L), Bacto Peptone (20 g/L), maltose (30 g/L), and lysine (2 g/L).

A chemically defined basal media called Bird Seed Medium (BSM, a modification of Bird Medium²⁵) was used in the Beacon and microplate culture models and contained KH₂PO₄ (8 g/L), (NH₃)₂SO₄ (15 g/L), MgSO₄ (25 mM), succinic acid (50 mM), EDTA (400 uM), ZnSO₄ (200 μM), CuSO₄ (20 μM), MnCl₂ (16 μM), CoCl₂ (20 μM), Na₂MoO₄ (20 μM), FeSO₄ (100 μM), CaCl₂) (200 μM), biotin (0.6 mg/L), p-aminobenzoic acid (2.4 mg/L), calcium pantothenate (12 mg/L), nicotinic acid (12 mg/L), myoinositol (300 mg/L), thiamine·HCl (12 mg/L), and pyridoxine·HCl (12 mg/L), adjusted to pH 5.0. Sucrose, glucose, fructose, maltose, and/or lysine were included in various concentrations in optimization experiments.

### VI. PARTIAL LISTING OF EMBODIMENTS

The following numbered embodiments are provided herein:

Embodiment 1. A method for evaluating bioproductivity of a cell, the method including: disposing a cell into a chamber of a microfluidic device, the microfluidic device having a microfluidic circuit including a flow region and the chamber, where the chamber includes an opening to the flow region; forming an in situ-generated barrier within the chamber, where the in situ-generated barrier defines an enclosed culture area within the chamber for culturing the cell; allowing the cell to secrete an analyte within the enclosed culture area; introducing a first fluidic medium including a reporter molecule into the flow region of the microfluidic circuit, where the reporter molecule is configured to bind to the analyte to form a reporter molecule: secreted analyte complex (RMSA complex), where the reporter molecule includes a first detectable label; and detecting a first signal associated with the first detectable label within an area of interest within the microfluidic circuit, thereby evaluating the bioproductivity of the cell.

Embodiment 2. The method of embodiment 1, where the in situ-generated barrier has a first permeability with respect to the analyte and a second permeability with respect to the reporter molecule, and where the first permeability is lower than the second permeability.

Embodiment 3. The method of embodiment 1 or 2, where the in situ-generated barrier has a porosity that impedes diffusion of the RMSA complex through the in situ-generated barrier.

Embodiment 4. The method of embodiment 3, where the porosity of the in situ-generated barrier substantially prevents diffusion of the RMSA complex through the in situ-generated barrier.

Embodiment 5. The method of any one of embodiments 1 to 4, where the area of interest is within the enclosed culture area.

Embodiment 6. The method of any one of embodiments 1 to 3, where the in situ-generated barrier has a porosity that allows diffusion of the RMSA complex through the in situ-generated barrier.

Embodiment 7. The method of any one of embodiments 1 to 6, where the in situ-generated barrier includes a gap through which the RMSA complex can diffuse (e.g., and thereby cross the in situ-generated barrier).

Embodiment 8. The method of any one of embodiments 1 to 4 or 6 to 7, where the area of interest is located within the chamber but not within the enclosed culture area.

Embodiment 9. The method of embodiment 10, where the area of interest is within a cell-free region of the chamber.

Embodiment 10. The method of any one of embodiments 1 to 9, where the in situ-generated barrier includes one or more discrete sections, each of which is moveably connected to one or more surfaces of the chamber, where application of a threshold pressure to the one or more discrete sections of the in situ-generated barrier moves at least one of the one or more discrete sections with respect to the one or more surfaces of the chamber and thereby creates an opening in the enclosed culture area.

Embodiment 11. The method of embodiment 10, where the in situ-generated barrier includes two or more discrete sections, where adjacent sections are separated from one another by a gap.

Embodiment 12. The method of embodiment 10 or embodiment 11, where the in situ-generated barrier consists of (or consists essentially of) two discrete sections which are separated from one another by a gap.

Embodiment 13. The method of any one of embodiments 1 to 12, where a portion of the in situ-generated barrier has a thickness that is smaller than the height of the chamber.

Embodiment 14. The method of any one of embodiments 10 to 13, where the in situ-generated barrier includes a non-uniform thickness with respect to an axis of the chamber such that a portion of the in situ-generated barrier is less thick than other portions of the in situ-generated barrier.

Embodiment 15. The method of embodiment 14, where the less thick portion of the in situ-generated barrier has a thickness that is smaller than the height of the chamber.

Embodiment 16. The method of any one of embodiments 1 to 16, where allowing the cell to secrete an analyte includes inducing the cell to secrete an analyte.

Embodiment 17. The method of embodiment 16, where inducing the cell to secrete an analyte includes introducing a second fluidic medium oxygenated with at least about 10%, 15%, 20%, 25%, 30%, 35%, 40%, 45%, 50%, 55%, or 60% of oxygen.

Embodiment 18. The method of any one of embodiments 1 to 17, where the in situ-generated barrier has a porosity that substantially prevents the cell from crossing through the in situ-generated barrier.

Embodiment 19. The method of any one of embodiments 1 to 18, where the reporter molecule further includes a binding component configured to bind the analyte.

Embodiment 20. The method of embodiment 19, where the binding component of the reporter molecule includes an amino acid, a polypeptide, a nucleotide, a nucleic acid, or a combination thereof.

Embodiment 21. The method of embodiment 20, where the binding component of the reporter molecule includes a protein.

Embodiment 22. The method of any one of embodiments 1 to 21, where the first detectable label includes a visible, luminescent, phosphorescent, or fluorescent detectable label.

Embodiment 23. The method of embodiment 22, where the first signal associated with the first detectable label includes a fluorescent signal, a luminescent signal, a visible signal, or a phosphorescent signal.

Embodiment 24. The method of any one of embodiments 1 to 23, where introducing the first fluidic medium including the reporter molecule includes allowing the reporter molecule to diffuse into the chamber.

Embodiment 25. The method of embodiment 24, where allowing the reporter molecule to diffuse into the chamber includes allowing the reporter molecule to reach an equilibrium between the flow region and the chamber.

Embodiment 26. The method of any one of embodiments 1 to 25, where the first signal associated with the first detectable label is detected after a steady state equilibrium is reached.

Embodiment 27. The method of any one of embodiments 1 to 26, where the first signal associated with the first detectable label is detected while perfusing a second fluidic medium into the flow region, where the second fluidic medium does not include the reporter molecule.

Embodiment 28. The method of any one of embodiments 1 to 27, further including normalizing the detected first signal associated with the first detectable label with a biomass of the cell.

Embodiment 29. The method of embodiment 30, where the biomass is measured by taking a brightfield image of the microfluidic circuit before introducing the first fluidic medium including the reporter molecule; and measuring an optical density from the brightfield image, where the optical density is measured in a selected area including the biomass.

Embodiment 30. The method of embodiment 29, where the optical density score corresponds to the measured biomass.

Embodiment 31. The method of any one of embodiments 1 to 30, further including: introducing a reference molecule into the flow region, where the reference molecule includes a second detectable label different from the first detectable label, and further where the reference molecule does not bind the analyte; allowing the reference molecule to diffuse into the chamber; and detecting a reference signal associated with the second detectable label.

Embodiment 32. The method of embodiment 31, where the second detectable label includes a visible, luminescent, phosphorescent, or fluorescent detectable label.

Embodiment 33. The method of embodiment 31 or embodiment 32, where the second signal associated with the second detectable label is detected after a steady state equilibrium is reached.

Embodiment 34. The method of any one of embodiments 31 to 33, further including normalizing the first signal associated with the first detectable label with the reference signal associated with the second detectable label.

Embodiment 35. The method of any one of embodiments 1 to 34, where the chamber is a first chamber of the microfluidic device, and the microfluidic device further includes a second chamber.

Embodiment 36. The method of embodiment 35, where disposing a cell into the chamber includes: disposing a first cell into the first chamber and disposing a second cell into the second chamber; and detecting a first signal associated with the first detectable label includes detecting a first signal associated with the first detectable label in the first chamber and detecting a second signal associated with the first detectable label in the second chamber.

Embodiment 37. The method of embodiment 36, further including: comparing the first signal of the first chamber and the second signal of the second chamber, and selecting the first cell or the second cell based on the comparison; or comparing the first signal and/or the second signal with a threshold, and selecting the first cell and/or the second cell based on the comparison.

Embodiment 38. The method of any one of embodiments 1 to 37, further including exporting the cell from the chamber and, optionally, from the microfluidic device.

Embodiment 39. The method of embodiment 38, where exporting the cell includes directing a laser illumination upon a selected area of the chamber to create a bubble pushing the cell toward the opening of the chamber.

Embodiment 40. The method of any one of embodiments 1 to 39, where the flow region includes a microfluidic channel, and the opening of the chamber is proximal to the microfluidic channel and oriented substantially parallel to a direction of flow of a fluidic medium in the microfluidic channel (e.g., when the fluidic medium is flowing in the microfluidic channel).

Embodiment 41. The method of any one of embodiments 1 to 40, where the chamber includes an isolation region and a connection region fluidically connecting the isolation region to the flow region; and where the connection region includes the opening to the flow region.

Embodiment 42. The method of embodiment 41, where the enclosed culture area is within the isolation region.

Embodiment 43. The method of any one of embodiments 1 to 42, where the in situ-generated barrier includes a solidified polymer network.

Embodiment 44. The method of embodiment 43, where the solidified polymer network includes a synthetic polymer, a modified synthetic polymer, or a biological polymer.

Embodiment 45. The method of embodiment 44, where the solidified polymer network includes at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination.

Embodiment 46. The method of any one of embodiments 1 to 45, where the cell is a eukaryotic cell or a prokaryotic cell.

Embodiment 47. The method of embodiment 46, where the cell is an animal cell, a plant cell, or a bacteria cell.

Embodiment 48. The method of embodiment 47, where the cell is a fungus cell.

Embodiment 49. The method of embodiment 48, where the cell is a yeast cell.

Embodiment 50. The method of embodiment 49, where the yeast cell is a Saccharomyces cell (e.g., Saccharomyces cerevisiae) or a Pichia cell (e.g., Pichia pastoris).

Embodiment 51. The method of any one of embodiments 1 to 50, where the analyte is an amino acid, a polypeptide, a nucleotide, a nucleic acid, or a combination.

Embodiment 52. A method for evaluating bioproductivity of a cell, the method including: disposing the cell into a chamber of a microfluidic device, the microfluidic device having a microfluidic circuit including a flow region and the chamber, where the chamber including an opening to the flow region; forming an in situ-generated barrier within the chamber, where the in situ-generated barrier defines within the chamber an assay area and an enclosed culture area for culturing the cell; disposing a micro-object in the assay area of the chamber, where the micro-object includes a capture moiety configured to bind an analyte secreted by the cell; allowing the cell to secreting the analyte; introducing a first fluidic medium including a reporter molecule into the flow region, where the reporter molecule includes a first detectable label and a binding component configured to bind to the analyte; and detecting a first signal associated with the first detectable label within an area of interest within the microfluidic circuit, thereby evaluating the bioproductivity of the cell.

Embodiment 53. The method of embodiment 52, where the in situ-generated barrier has a porosity that allows diffusion of the analyte through the in situ-generated barrier.

Embodiment 54. The method of embodiment 52 or embodiment 53, where the in situ-generated barrier includes a gap through which the analyte can diffuse (e.g., and thereby cross the in situ-generated barrier).

Embodiment 55. The method of any of one of embodiments 52 to 54, where the in situ-generated barrier has a porosity that substantially prevents the cell from crossing through the in situ-generated barrier.

Embodiment 56. The method of any one of embodiments 52 to 55, where the in situ-generated barrier includes one or more discrete sections, each of which is moveably connected to one or more surfaces of the chamber, where application of a threshold pressure to the one or more discrete sections of the in situ-generated barrier moves at least one of the one or more discrete sections with respect to the one or more surfaces of the chamber and thereby creates an opening in the enclosed culture area.

Embodiment 57. The method of embodiment 56, where the in situ-generated barrier includes two or more discrete sections, where adjacent sections are separated from one another by a gap.

Embodiment 58. The method of embodiment 56 or embodiment 57, where the in situ-generated barrier consists of (or consists essentially of) two discrete sections which are separated from one another by a gap.

Embodiment 59. The method of any one of embodiments 52 to 58, where a portion of the in situ-generated barrier has a thickness that is smaller than the height of the chamber.

Embodiment 60. The method of any one of embodiments 56 to 59, where the in situ-generated barrier includes a non-uniform thickness with respect to an axis of the chamber such that a portion of the in situ-generated barrier is less thick than other portions of the in situ-generated barrier.

Embodiment 61. The method of embodiment 60, where the less thick portion of the in situ-generated barrier has a thickness that is smaller than the height of the chamber.

Embodiment 62. The method of any one of embodiments 52 to 61, where, after disposing a micro-object in the assay area of the chamber, the method further includes introducing a culture medium and culturing the cell within the chamber in the culture medium before allowing the cell to secreting the analyte.

Embodiment 63. The method of any one of embodiments 52 to 62, where allowing the cell to secrete an analyte includes inducing the cell to secrete an analyte.

Embodiment 64. The method of embodiment 63, where inducing the cell to secrete an analyte includes introducing a second fluidic medium oxygenated with at least about 10%, 15%, 20%, 25%, 30%, 35%, 40%, 45%, 50%, 55%, or 60% of oxygen.

Embodiment 65. The method of any one of embodiments 52 to 64, where the area of interest is in the assay area.

Embodiment 66. The method of embodiment 65, where the area of interest is within a cell-free region of the chamber.

Embodiment 67. The method of any one of embodiments 52 to 66, where the capture moiety includes a peptide or a protein.

Embodiment 68. The method of any one of embodiments 52 to 67, where the analyte includes a first tag configured to be bound by the capture moiety of the micro-object and a second tag configured to be bound by the binding component of the reporter molecule.

Embodiment 69. The method of embodiment 68, where the first tag includes a FLAG-tag, His tag, E-tag, Myc-tag, T7, NE-tag, Spot-tag, V5-tag, VSV-tag, or a combination thereof.

Embodiment 70. The method of any one of embodiments 52 to 69, where micro-object is a bead.

Embodiment 71. The method of any one of embodiments 52 to 70, where the binding component of the reporter molecule includes an amino acid, a polypeptide, a nucleotide, a nucleic acid, or a combination thereof.

Embodiment 72. The method of embodiment 71, where the binding component of the reporter molecule includes a protein.

Embodiment 73. The method of any one of embodiments 52 to 72, where the first detectable label includes a visible, luminescent, phosphorescent, or fluorescent detectable label.

Embodiment 74. The method of embodiment 73, where the first signal associated with the first detectable label includes a fluorescent signal, a luminescent signal, a visible signal, or a phosphorescent signal.

Embodiment 75. The method of any one of embodiments 52 to 74, further including normalizing the first signal associated with the first detectable label with a biomass of the cell.

Embodiment 76. The method of embodiment 75, where the biomass is measured by: taking a brightfield image of the chamber before introducing the first fluidic medium including the reporter molecule; and measuring an optical density from the brightfield image, where the optical density is measured in a selected area including the biomass.

Embodiment 77. The method of embodiment 76, where the optical density score corresponds to the measured biomass.

Embodiment 78. The method of any one of embodiments 52 to 77, where the chamber is a first chamber of the microfluidic device, and the microfluidic device further includes a second chamber.

Embodiment 79. The method of embodiment 78, where disposing the cell into a chamber of a microfluidic device includes: disposing a first cell into the first chamber and disposing a second cell into the second chamber; and detecting a first signal associated with the first detectable label includes detecting a first signal associated with the first detectable label in the first chamber and detecting a second signal associated with the first detectable label in the second chamber.

Embodiment 80. The method of embodiment 79, further including: comparing the first signal of the first chamber and the second signal of the second chamber, and selecting the first cell or the second cell based on the comparison; or comparing the first signal and/or the second signal with a threshold, and selecting the first cell and/or the second cell based on the comparison.

Embodiment 81. The method of any one of embodiments 52 to 80, further including exporting the cell from the chamber and, optionally, from the microfluidic device.

Embodiment 82. The method of embodiment 80, exporting the cell from the chamber includes directing a laser illumination upon a selected area of the chamber to create a bubble pushing the cell toward the opening of the chamber.

Embodiment 83. The method of any one of embodiments 52 to 82, where the flow region includes a microfluidic channel, and the opening of the chamber is proximal to the microfluidic channel and oriented substantially parallel to a direction of flow of a fluidic medium in the microfluidic channel (e.g., when the fluidic medium is flowing in the microfluidic channel).

Embodiment 84. The method of any one of embodiments 52 to 83, where the chamber includes an isolation region and a connection region fluidically connecting the isolation region to the flow region; and where the connection region includes the opening to the flow region.

Embodiment 85. The method of embodiment 84, where the enclosed culture area is within the isolation region.

Embodiment 86. The method of any one of embodiments 52 to 85, where the in situ-generated barrier includes a solidified polymer network.

Embodiment 87. The method of embodiment 86, where the solidified polymer network includes a synthetic polymer, a modified synthetic polymer, or a biological polymer.

Embodiment 88. The method of embodiment 87, where the solidified polymer network includes at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination.

Embodiment 89. The method of any one of embodiments 52 to 88, where the cell is a eukaryotic cell or a prokaryotic cell.

Embodiment 90. The method of embodiment 89 where the cell is an animal cell, a plant cell, or a bacteria cell.

Embodiment 91. The method of embodiment 90, where the cell is a fungus cell.

Embodiment 92. The method of embodiment 91, where the cell is a yeast cell.

Embodiment 93. The method of embodiment 92, where the yeast cell is a Saccharomyces cell (e.g., Saccharomyces cerevisiae) or a Pichia cells (e.g., Pichia pastoris).

Embodiment 94. A kit for evaluating bioproductivity of a cell, the kit including: a reporter molecule including a first detectable label and a binding component configured to bind an analyte secreted by a cell to form a reporter molecule: secreted analyte complex (RMSA complex); and a prepolymer configured to be controllably activated to form an in situ-generated barrier including a solidified polymer network, where the in situ-generated barrier has a porosity that substantially prevents the cell from crossing through the in situ-generated barrier.

Embodiment 95. The kit of embodiment 94, where the in situ-generated barrier has a first permeability with respect to the analyte and a second permeability with respect to the reporter molecule, and where the first permeability is lower than the second permeability.

Embodiment 96. The kit of embodiment 94 or 95, where the porosity of the in situ-generated barrier impedes diffusion of the RMSA complex through the in situ-generated barrier.

Embodiment 97. The kit of any one of embodiments 94 to 96, where the porosity of the in situ-generated barrier substantially prevents diffusion of the RMSA complex through the in situ-generated barrier.

Embodiment 98. The kit of any one of embodiments 94 to 96, where the porosity of the in situ-generated barrier allows the RMSA complex to diffuse through the in situ-generated barrier.

Embodiment 99. The kit of any one of embodiments 94 to 98, where the binding component of the reporter molecule includes an amino acid, a peptide, a protein, a nucleotide, a nucleic acid, or any combination thereof.

Embodiment 100. The kit of embodiment 99, where the binding component of the reporter molecule includes a protein.

Embodiment 101. The kit of any one of embodiments 94 to 100, where the first detectable label includes a visible, luminescent, phosphorescent, or fluorescent detectable label.

Embodiment 102. The kit of any one of embodiments 94 to 101, further including a micro-object including a capture moiety configured to bind the analyte.

Embodiment 103. The kit of 102, where the capture moiety includes a peptide or a protein.

Embodiment 104. The kit of embodiment 102 or embodiment 103, where the analyte includes a first tag configured to be bound by the capture moiety of the micro-object and a second tag configured to be bound by the binding component of the reporter molecule.

Embodiment 105. The kit of embodiment 104, where the first tag includes a FLAG-tag, His tag, E-tag, Myc-tag, T7, NE-tag, Spot-tag, V5-tag, VSV-tag, or a combination thereof.

Embodiment 106. The kit of any one of embodiments 102 to 105, where micro-object is a bead.

Embodiment 107. The kit of any one of embodiments 94 to 106, further including a reference molecule including a second detectable label different from the first detectable label, and further where the reference molecule does not bind the analyte.

Embodiment 108. The kit of embodiment 107, where the second detectable label includes a visible, luminescent, phosphorescent, or fluorescent detectable label.

Embodiment 109. The kit of any one of embodiments 94 to 108, further including a microfluidic device including a microfluidic circuit including a flow region and a chamber, where the chamber includes an opening to the flow region.

Embodiment 110. The kit of embodiment 109, where the flow region includes a microfluidic channel, and the opening of the chamber is proximal to the microfluidic channel and oriented substantially parallel to a flow of a fluidic medium in the microfluidic channel, when the fluidic medium is flowing in the microfluidic channel.

Embodiment 111. The kit of embodiment 109 or embodiment 110, where the chamber includes an isolation region and a connection region fluidically connecting the isolation region to the flow region; and where the connection region includes the opening to the flow region.

Embodiment 112. The kit of any one of embodiments 109 to 111, where the microfluidic device includes a plurality of chambers.

Embodiment 113. The kit of any one of embodiments 109 to 112, where the microfluidic device includes a substrate configured to generate dielectrophoresis (DEP) forces within the microfluidic circuit.

Embodiment 114. The kit of any one of embodiments 94 to 113, where the solidified polymer network includes a synthetic polymer, a modified synthetic polymer, or a biological polymer.

Embodiment 115. The kit of embodiment 114, where the solidified polymer network includes at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination.

Embodiment 116. The kit of any one of embodiments 94 to 115, where the cell is a eukaryotic cell or a prokaryotic cell.

Embodiment 117. The kit of embodiment 116, where the cell is an animal cell, a plant cell, or a bacteria cell.

Embodiment 118. The kit of embodiment 117, where the cell is a fungus cell.

Embodiment 119. The kit of embodiment 118, where cell is a yeast cell.

Embodiment 120. The kit of embodiment 119, where the yeast cell is a Saccharomyces cell (e.g., Saccharomyces cerevisiae) or a Pichia cells (e.g., Pichia pastoris).

Embodiment 121. A method for biomass measurement in a microfluidic device, the method including: obtaining a microfluidic device including a chamber having a biomass to be measured, where the microfluidic device includes a microfluidic circuit including a flow region and a chamber fluidically connected to the flow region, where the chamber includes an opening to the flow region; obtaining a first brightfield image of the chamber or a first area thereof including the biomass; and measuring a first optical density score from the first brightfield image.

Embodiment 122. The method of embodiment 121, where the first optical density score corresponds to the measured biomass.

Embodiment 123. The method of embodiment 121 or 122, where the biomass includes one or more cells.

Embodiment 124. The method of any one of embodiments 121 to 123, where the flow region includes a microfluidic channel, and the opening of the chamber is proximal to the microfluidic channel and oriented substantially parallel to a flow of a fluidic medium in the microfluidic channel, when the fluidic medium is flowing in the microfluidic channel.

Embodiment 125. The method of any one of embodiments 121 to 124, where the chamber includes an isolation region and a connection region fluidically connecting the isolation region to the flow region; and where the connection region includes the opening to the flow region.

Embodiment 126. The method of embodiment 125, where the first area of the chamber is within the isolation region.

Embodiment 127. The method of any one of embodiments 121 to 126, where obtaining the microfluidic device including the biomass to be measured includes: disposing a cell into the chamber and expanding the cell into a clonal population within the chamber.

Embodiment 128. The method of embodiment 126 or embodiment 127, further including, before introducing the cell into the chamber, obtaining a reference brightfield image of the microfluidic circuit and measuring a reference optical density score of the chamber from the reference brightfield image.

Embodiment 129. The method of embodiment 128, further including normalizing the first optical density score with the reference optical density score.

Embodiment 130. The method of any one of embodiments 121 to 130, further including: selecting a second area in the microfluidic circuit, where the second area does not include the biomass; measuring a second optical density score within the second selected area; and correcting the first optical density with the second optical density score.

Embodiment 131. The method of embodiment 130, where the second area is within a microfluidic channel of the microfluidic device or within the chamber.

Embodiment 132. The method of embodiment 131, where the chamber is a first chamber of the microfluidic device, and the microfluidic device includes a second chamber, and further where the second chamber does not include a cell, and the second area is within the second chamber.

Embodiment 133. The method of any one of embodiments 121 to 132, further including concentrating biomass to obtain a consolidated area containing the biomass, and where the first area is within the consolidated area.

Embodiment 134. The method of embodiment 133, where concentrating the biomass is performed by centrifugation.

Embodiment 135. The method of any one of embodiments 121 to 134, where the cell is a eukaryotic cell or a prokaryotic cell.

Embodiment 136. The method of embodiment 135, where the cell is an animal cell, a plant cell, or a bacteria cell.

Embodiment 137. The method of embodiment 136, where the cell is a fungus cell.

Embodiment 138. The method of embodiment 137, where the cell is a yeast cell.

Embodiment 139. The method of embodiment 138, where the yeast cell is a Saccharomyces cell (e.g., Saccharomyces cerevisiae) or a Pichia cells (e.g., Pichia pastoris). Embodiment 140. The method of any one of embodiments 1 to 51, wherein allowing the cell to secrete an analyte comprises inducing the cell to secrete an analyte by introducing a second fluidic medium comprising at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, or about 15% methanol v/v.

Embodiment 141. The method of any one of embodiments 51 to 93, wherein allowing the cell to secrete an analyte comprises inducing the cell to secrete an analyte by introducing a second fluidic medium comprising at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, or about 15% methanol v/v.

Embodiment 201. A method for improving yeast cell bioproductivity including; culturing one or more yeast cells within each of a plurality of chambers of a microfluidic device, where the microfluidic device includes a flow region configured to flow a first fluidic medium and the chamber opens to the flow region; expanding the one or more yeast cells to form a population of yeast cells in each of the plurality of chambers; monitoring the production of a biomolecule by the population of yeast cells in each of the plurality of chambers; and predicting one or more populations of yeast cells configured to effectively produce the biomolecules.

Embodiment 202. The method of embodiment 201, where effectively producing the biomolecules includes effectively producing the biomolecules when culturing the population of yeast cells in a macroscale reactor.

Embodiment 203. The method of embodiment 201 or 202, where predicting further includes selecting one or more populations of yeast cells producing higher levels of the biomolecule relative to levels of production of the biomolecule of the population of yeast cells in the other of the plurality of chambers.

Embodiment 204. The method of any one of embodiments 201-203, where the biomolecule is a small organic molecule having a molecular weight less than 2000 Da.

Embodiment 205. The method of any one of embodiments 201-204, where monitoring the production of the biomolecule includes detecting a detectable signal associated with the biomolecule.

Embodiment 206. The method of any one of embodiments 201-205, where the biomolecule is inherently detectable.

Embodiment 207. The method of any one of embodiments 201-205, where the biomolecule is detectable upon labelling.

Embodiment 208. The method of any one of embodiments 201-207, where culturing in the chamber of the microfluidic device includes culturing the population of yeast cells within a volume of medium less than 5 nanoliters.

Embodiment 209. The method of any one of embodiments 202-208, where the macroscale reactor includes a volume of 100 mL, 1 L, 10 L, 100 L or more.

Embodiment 210. The method of any one of embodiments 201-209, where culturing in the chamber of the microfluidic device includes culturing under substantially similar conditions to conditions of culturing in the macroscale reactor.

Embodiment 211. The method of any one of embodiments 201-210, further including monitoring the production of the biomolecule at a time where the culturing conditions approximate pseudo steady state conditions.

Embodiment 212. The method of any one of embodiments 201-211, further including calculating a relative specific productivity for each population of yeast cells in each chamber of the plurality of chamber.

Embodiment 213. The method of embodiment 212, where calculating the relative specific productivity includes normalizing the detectable signal for each chamber by a measured biomass of each chamber.

Embodiment 214. The method of any one of embodiments 201-213, where effectively producing the biomolecules includes effectively producing the biomolecules based on feedstock.

Embodiment 215. The method of embodiment 214, where effectively producing the biomolecules based on feedstock includes at least one of efficiently converting the feedstock to biomolecules and efficiently producing the biomolecules in the presence of byproduct accumulation.

Embodiment 216. The method of any one of embodiments 201-215, where the population of yeast cells is a clonal population of yeast cells.

Embodiment 217. A method for assessing relative productivity of a detectable molecule by a population of yeast cells, in a microfluidic device having an enclosure including a channel and a plurality of chambers, each chamber of the plurality having an opening fluidically connecting the chamber to the channel, the method including: disposing a yeast cell configured to produce the detectable molecule in each chamber of the plurality of chambers; flowing a first aqueous medium into the channel; culturing the yeast cell to expand to a clonal population of yeast cells; flowing a water immiscible fluidic medium into the channel, displacing substantially all of the first aqueous medium in the channel; monitoring over a period of time an increase of a signal from the detectable molecules produced by the clonal population of yeast cells in each chamber of the plurality; and determining a relative productivity for each clonal population of yeast cells.

Embodiment 218. The method of embodiment 217, further including subsequently flowing a second aqueous medium into the channel, thereby displacing the water immiscible fluidic medium from the channel.

Embodiment 219. The method of embodiment 218, further including flowing a third aqueous medium including a surfactant into the channel, thereby clearing a residual portion of the water immiscible medium from the channel.

Embodiment 220. The method of embodiment 219, further including unpenning a selected clonal population of yeast cells out of the chamber and exporting the selected clonal population of yeast cells out of the microfluidic device.

Embodiment 221. The method of any one of embodiments 217-220, where displacing substantially all of the first aqueous medium in the channel by the water immiscible fluidic medium is performed without displacing the first aqueous medium in the chambers of the plurality of chambers

Embodiment 222. The method of any one of embodiments 217-221, where determining the relative productivity includes determining a relative productivity when the clonal population is cultured in the presence of a selected level of a component of the first aqueous medium.

Embodiment 223. The method of embodiment 222, where the component is a nutrient for the clonal population of yeast cells.

Embodiment 224. The method of embodiment 222 or 223, where the selected level of the component is a growth limiting level of the component.

Embodiment 225. The method of any one of embodiments 217-224, where determining the relative productivity includes determining a relative productivity of the clonal population in the presence of increasing levels of byproducts from the clonal population.

Embodiment 226. The method of any one of embodiments 217-225, where the molecule is inherently detectable.

Embodiment 227. The method of any one of embodiments 217-225, where the molecule is detectable upon labelling.

Embodiment 228. The method of any one of embodiments 217-227, where the period of time is about 10 min, about 20 min, about 30 min, about 40 min, or about 50 min.

Embodiment 229. The method of any one of embodiment 217-228, where the method further includes normalizing the increase of the signal from the detectable molecules to a biomass of the clonal population of the yeast cells.

Embodiment 230. A method for assessing relative productivity of a detectable molecule by a population of yeast cells, in a microfluidic device having an enclosure including a channel and a plurality of chambers, each chamber of the plurality having an opening fluidically connecting the chamber to the channel, the method including: disposing a yeast cell configured to produce the detectable molecule in each chamber of the plurality of chambers; perfusing an aqueous medium through the channel; culturing the yeast cell to expand to a clonal population of yeast cells; increasing a rate of perfusing the aqueous medium for a selected first period of time thereby establishing a substantially steady state of diffusion of the detectable molecules from each chamber into the channel; imaging a signal from the detectable molecules produced by the clonal population of yeast cells in each chamber of the plurality; and determining a relative productivity for each clonal population of yeast cells.

Embodiment 231. The method of embodiment 230, where the clonal population of yeast cells is cultured in the presence of a selected level of a component of the first aqueous medium.

Embodiment 232. The method of embodiment 232, where the component is a nutrient for the clonal population of yeast cells.

Embodiment 233. The method of embodiment 231 or 232, where the selected level of the component is a growth limiting level of the component.

Embodiment 234. The method of any one of embodiments 230-233, where the molecule is inherently detectable.

Embodiment 235. The method of any one of embodiments 230-233, where the molecule is detectable upon labelling.

Embodiment 236. The method of any one of embodiments 230-235, where the selected first period of time is about 5 min.

Embodiment 237. The method of any one of embodiments 230-236, where the rate of perfusing the aqueous medium is increased from a first rate of flow to a second rate of flow by a factor of about 2 to about 10 for the selected period of time.

Embodiment 238. The method of embodiment 237, further including reducing the rate of perfusion to the first rate of flow for a second selected period of time; increasing the flow to the second rate of flow for the selected first period of time, thereby establishing the substantially steady state of diffusion of the detectable molecules; and imaging the signal from the detectable molecules produced by the clonal population of yeast cells at a second point in time.

Embodiment 239. The method of any one of embodiments 230-238, where determining the relative productivity includes determining a relative productivity of the clonal population when culturing in the presence of increasing levels of byproducts from the clonal population.

Embodiment 240. The method of any one of embodiments 230-239, where the method further includes normalizing the increase of the signal from the detectable molecules to a biomass of the clonal population of the yeast cells.

Embodiment 301. A method for distributing biological micro-objects in a microfluidic device, where the microfluidic device includes a flow region including a flow channel and at least one chamber fluidically connecting to the flow channel; where the method includes: disposing at least one biological micro-object within a chamber; incubating the at least one biological micro-object thereby forming a population of cells; and centrifuging the microfluidic device to redistribute at least a portion of the population of cells within the microfluidic device.

Embodiment 302. The method of embodiment 301, where the at least one chamber is a sequestration pen including an isolation region and a connection region fluidically connecting the isolation region to the flow channel.

Embodiment 303. The method of embodiment 302, where disposing including disposing the at least one biological micro-object within the isolation region.

Embodiment 304. The method of embodiment 302 or 303, where the population of cells is a clonal population of cells.

Embodiment 305. The method of any one of embodiments 302-304, where centrifuging redistributes at least a portion of the population of cells to a location within the isolation region.

Embodiment 306. The method of any one of embodiments 302-304, where the centrifuging redistributes at least a portion of the population of cells to a location within the connection region.

Embodiment 307. The method of any one of embodiments 302-304, where the centrifuging redistributes at least a portion of the population of cells to the flow channel.

Embodiment 308. The method of any one of embodiments 301-307, further including measuring an intensity of brightness of the chamber in a bright-field image.

Embodiment 309. The method of embodiment 308, where measuring is performed before disposing the at least one biological micro-object, after disposing the at least one biological micro-object, or both.

Embodiment 310. The method of embodiment 308, where measuring is performed before centrifuging the population of cells, after centrifuging the population of cells, or both.

Embodiment 311. The method of embodiment 308, where a first value of the intensity is obtained before disposing the at least one biological micro-object,

Embodiment 312. The method of embodiment 311, where a second value of the intensity is obtained after disposing the at least one biological micro-object.

Embodiment 313. The method of embodiment 312, where the second value of the intensity is obtained after centrifuging the population of cells.

Embodiment 314. The method of embodiment 312 or 313, where the first value and the second value are obtained under a substantially same illumination condition.

Embodiment 315. The method of any one of embodiments 308-314, further including normalizing the intensity with a background intensity; where the background intensity is obtained by measuring a brightness of an empty sequestration pen in the bright-field image.

Embodiment 316. The method of any one of embodiments 301-315, where the biological micro-object is a eukaryotic cell, a prokaryotic cell, or a combination thereof.

Embodiment 317. The method of any one of embodiments 301-315, where the biological micro-object is a mammalian cell, a bacterial cell, a fungal cell, a protozoan cell, or a combination thereof.

Embodiment 318. A method for biomass measurement in a microfluidic device, where the microfluidic device includes a flow region including a flow channel and at least one chamber fluidically connecting to the flow channel, where the method including: disposing at least one biological micro-object within the chamber; and measuring an intensity (It) of brightness of the chamber in a bright-field image, where the intensity represents a biomass of the at least one biological micro-object.

Embodiment 319. The method of embodiment 318, where the at least one chamber is a sequestration pen including an isolation region and a connection region fluidically connecting the isolation region to the flow channel.

Embodiment 320. The method of embodiment 319, where at least one biological micro-object is disposed in the isolation region.

Embodiment 321. The method of any one of embodiments 318-320, further including centrifuging the microfluidic device to provide a consolidated area populated by the at least one biological micro-object within the chamber.

Embodiment 322. The method of embodiment 321, where centrifuging is performed before the measuring.

Embodiment 323. The method of any one of embodiments 319-322, further including expanding the at least one biological micro-object to a population of cells within the isolation region.

Embodiment 324. The method of any one of embodiments 318-323, further including measuring a reference intensity (Iᵣ) of brightness of the chamber in a reference bright-field image before the at least one biological micro-objects are disposed.

Embodiment 325. The method of embodiment 324, where an optical density (OD) score is calculated by formula:

OD=1−Iₜ/I_{ref}

Embodiment 326. The method of embodiment 324 or 325, where the bright-field image and the reference bright-field image are obtained under a substantially same optical condition.

Embodiment 327. The method of embodiment 325 or 326, where the optical density score represents the biomass of the biological micro-objects, provided that the score is at least 0.08.

Embodiment 328. The method of embodiment 327, where the optical density score represents the biomass of the biological micro-objects, provided that the score is between 0.15 to 0.6.

Embodiment 329. The method of embodiment 328, where the optical density score represents the biomass of the biological micro-objects, provided that the score is between 0.15 to 0.3.

Embodiment 330. The method of any one of embodiments 318-329, further including normalizing the intensity (Iₜ) with a background intensity, where the background intensity is obtained by measuring a brightness of an empty sequestration in the bright-field image.

Embodiment 331. The method of any one of embodiments 324-330, further including normalizing the reference intensity (I_{ref}) with a background intensity, where the background intensity is obtained by measuring a brightness of an empty sequestration in the reference bright-field image.

Embodiment 332. A method for distributing biological micro-objects in a microfluidic device, including: providing a microfluidic device; where the microfluidic device includes a flow region including a flow channel and at least one chamber fluidically connecting to the flow channel; where at least one biological micro-object is disposed at a first location within the microfluidic device; and centrifuging the microfluidic device to redistribute the at least one biological micro-object from the first region to a second location within the microfluidic device.

Embodiment 333. The method of embodiment 332, where the at least one chamber is a sequestration pen including an isolation region and a connection region fluidically connecting the isolation region to the flow channel.

Embodiment 334. The method of embodiment 333, where the first location is within the isolation region, the connection region, or the flow channel.

Embodiment 335. The method of embodiment 333, where the first location is within the isolation region, and the at least one biological micro-object is disposed by loading the at least one biological micro-object through the flow channel and moving the at least one biological micro-object to the first location.

Embodiment 336. The method of any one of embodiments 333-335, where the second location is within the isolation region, the connection region, or the flow channel.

Embodiment 337. The method of any one of embodiments 332-336, where the at least one biological micro-object is a population of cells.

Embodiment 338. The method of any one of embodiments 332-337, where the biological micro-object is a eukaryotic cell, a prokaryotic cell, or a combination thereof.

Embodiment 339. The method of any one of embodiments 332-337, where the biological micro-object is a mammalian cell, a bacterial cell, a fungal cell, a protozoan cell, or a combination thereof.

1. A method for evaluating bioproductivity of a cell, the method comprising:
disposing a cell into a chamber of a microfluidic device, the microfluidic device having a microfluidic circuit comprising a flow region and the chamber, wherein the chamber comprises an opening to the flow region;
forming an in situ-generated barrier within the chamber, wherein the in situ-generated barrier defines an enclosed culture area within the chamber for culturing the cell;
allowing the cell to secrete an analyte within the enclosed culture area;
introducing a first fluidic medium comprising a reporter molecule into the flow region of the microfluidic circuit, wherein the reporter molecule is configured to bind to the analyte to form a reporter molecule: secreted analyte complex (RMSA complex), wherein the reporter molecule comprises a first detectable label; and
detecting a first signal associated with the first detectable label within an area of interest within the microfluidic circuit, thereby evaluating the bioproductivity of the cell.

2. The method of claim 1, wherein the in situ-generated barrier has a first permeability with respect to the analyte and a second permeability with respect to the reporter molecule, and wherein the first permeability is lower than the second permeability.

3. The method of claim 1, wherein the in situ-generated barrier has a porosity that impedes or substantially prevents diffusion of the RMSA complex through the in situ-generated barrier.

4. (canceled)

5. (canceled)

6. The method of claim 1, wherein the in situ-generated barrier has a porosity that allows diffusion of the RMSA complex through the in situ-generated barrier.

7. The method of claim 1, wherein the in situ-generated barrier comprises a gap through which the RMSA complex can diffuse.

8. The method of claim 1, wherein the area of interest is located within the chamber but not within the enclosed culture area, or wherein the area of interest is within a cell-free region of the chamber.

9. (canceled)

10. The method of claim 1, wherein the in situ-generated barrier comprises one or more discrete sections, each of which is moveably connected to one or more surfaces of the chamber, wherein application of a threshold pressure to the one or more discrete sections of the in situ-generated barrier moves at least one of the one or more discrete sections with respect to the one or more surfaces of the chamber and thereby creates an opening in the enclosed culture area.

11. (canceled)

12. The method of claim 10, wherein the in situ-generated barrier comprises a non-uniform thickness with respect to an axis of the chamber such that a portion of the in situ-generated barrier is less thick than other portions of the in situ-generated barrier, and wherein the less thick portion of the in situ-generated barrier has a thickness that is smaller than the height of the chamber.

13. (canceled)

14. (canceled)

15. The method of claim 1, wherein the in situ-generated barrier has a porosity that substantially prevents the cell from crossing through the in situ-generated barrier.

16. (canceled)

17. The method of claim 1, wherein the first detectable label comprises a visible, luminescent, phosphorescent, or fluorescent detectable label.

18. The method of claim 1, wherein introducing the first fluidic medium comprising the reporter molecule comprises allowing the reporter molecule to diffuse into the chamber.

19. The method of claim 1, wherein the first signal associated with the first detectable label is detected after a steady state equilibrium is reached.

20. The method of claim 1, wherein the first signal associated with the first detectable label is detected while perfusing a second fluidic medium into the flow region, wherein the second fluidic medium does not comprise the reporter molecule.

21. The method of claim 1, further comprising normalizing the detected first signal associated with the first detectable label with a biomass of the cell.

22. The method of claim 1, further comprising:
introducing a reference molecule into the flow region, wherein the reference molecule comprises a second detectable label different from the first detectable label, and further wherein the reference molecule does not bind the analyte;
allowing the reference molecule to diffuse into the chamber; and
detecting a reference signal associated with the second detectable label.

23. The method of claim 22, further comprising normalizing the first signal associated with the first detectable label with the reference signal associated with the second detectable label.

24. The method of claim 1, further comprising exporting the cell from the chamber from the microfluidic device.

25.-28. (canceled)

29. The method of claim 1, wherein the in situ-generated barrier comprises a solidified polymer network.

30. The method of claim 29, wherein the solidified polymer network comprises a synthetic polymer, a modified synthetic polymer, or a biological polymer.

31. The method of claim 1, wherein the analyte is an amino acid, a polypeptide, a nucleotide, a nucleic acid, or a combination thereof.

32. A method for evaluating bioproductivity of a cell, the method comprising:
disposing the cell into a chamber of a microfluidic device, the microfluidic device having a microfluidic circuit comprising a flow region and the chamber, wherein the chamber comprising an opening to the flow region;
forming an in situ-generated barrier within the chamber, wherein the in situ-generated barrier defines within the chamber an assay area and an enclosed culture area for culturing the cell;
disposing a micro-object in the assay area of the chamber, wherein the micro-object comprises a capture moiety configured to bind an analyte secreted by the cell;
allowing the cell to secreting the analyte;
introducing a first fluidic medium comprising a reporter molecule into the flow region, wherein the reporter molecule comprises a first detectable label and a binding component configured to bind to the analyte; and
detecting a first signal associated with the first detectable label within an area of interest within the microfluidic circuit, thereby evaluating the bioproductivity of the cell.

33.-52. (canceled)

53. A kit for evaluating bioproductivity of a cell, the kit comprising:
a reporter molecule comprising a first detectable label and a binding component configured to bind an analyte secreted by a cell to form a reporter molecule:
secreted analyte complex (RMSA complex); and
a prepolymer configured to be controllably activated to form an in situ-generated barrier comprising a solidified polymer network, wherein the in situ-generated barrier has a porosity that substantially prevents the cell from crossing through the in situ-generated barrier.

54.-57. (canceled)
